(19) United States
(12) Patent Application Publication
(10) Pub. No.: US 2024/0226314 A1
(43) Pub. Date: Jul. 11, 2024

(54) TRANSFERRIN RECEPTOR BINDING PROTEINS

(71) Applicant: Sanofi, Paris (FR)

(72) Inventors: Beatrice CAMERON, Paris (FR); Cécile CAPDEVILA, Choisy-le-Roi (FR); Tarik DABDOUBI, Favières (FR); Tuan-Minh DO, Sceaux (FR); Blandine DUPERRIER, Vitry-sur-Seine (FR); Marie GAGNAIRE, Alfortville (FR); Kelly GEORGE, Westborough, MA (US); Dominique LESUISSE, Montreuil (FR); Chiara RAPISARDA, Paris (FR); Fabienne SOUBRIER, Thiais (FR); Arjan VAN DER FLIER, Somerville, MA (US)

(73) Assignee: Sanofi, Paris (FR)

(21) Appl. No.: 18/529,691

(22) Filed: Dec. 5, 2023

(30) Foreign Application Priority Data
Dec. 5, 2022 (EP) 22306784.4

(51) Int. Cl. A61K 47/68; A61K 38/47; A61P 3/00; C07K 16/28

(52) U.S. Cl. CPC A61K 47/6815; A61K 38/47; A61K 47/6849; A61K 47/6889; A61P 3/00; C07K 16/2881; C12Y 302/0102

(57) ABSTRACT

The present disclosure provides antibodies and antigen-binding fragments that target transferrin receptor. Also provided is the use of these antibodies and antigen-binding fragments as carriers to deliver therapeutic molecules across the blood-brain barrier.

### CROSS-REFERENCE TO RELATED APPLICATION

This application claims priority to European Patent Application 22306784.4, filed Dec. 5, 2022. The disclosure of that priority application is incorporated by reference herein in its entirety.

### SEQUENCE LISTING

The instant application contains a Sequence Listing which has been submitted electronically in XML format and is hereby incorporated by reference herein in its entirety. The electronic copy of the Sequence Listing, created on Dec. 3, 2023, is named 122548.US005.xml and is 92,235 bytes in size.

### BACKGROUND OF THE INVENTION

Transferrin receptor 1 (TfR), also known as CD71, is a ubiquitously expressed transmembrane glycoprotein involved in cellular uptake of iron. TfR imports iron through receptor-mediated endocytosis of transferrin, an iron-binding protein. Since TfR is highly expressed by brain capillary endothelial cells forming the blood-brain barrier (BBB) and transports iron across the BBB through transcytosis, it has been explored as a potential target for molecular shuttles that are designed to transport large molecule drugs across the BBB (see, e.g., Bourassa et al., Mol Pharm. (2019) 16(2):583-94).

There are several notable challenges in using TfR as a target for molecular shuttles. First, the presence of high blood levels of transferrin could require the shuttles to compete with transferrin for binding to TfR. Other challenges include specificity for the brain tissue, potential lysosomal degradation, and significant transport into the brain parenchyma (Pulgar, Front Neurosci. (2019) 12: doi10.3389/fnins.2018.01019). There remains a need for TfR-targeting molecular shuttles that have sufficient brain specificity and efficient uptake.

### SUMMARY OF THE INVENTION

The present disclosure provides anti-human transferrin receptor (TfR) antibodies or antigen-binding fragments thereof, wherein the antibodies or antigen-binding fragments comprise a heavy chain CDR (HCDR) 1 comprising GYTFTRYY (SEQ ID NO: 26), or GYTFTRYW (SEQ ID NO: 27), or DYTFTRYW (SEQ ID NO: 5), an HCDR2 comprising IDPSVSET (SEQ ID NO: 28) or IDPSVSEC (SEQ ID NO: 6), and an HCDR3 comprising SQIRLPYYYAMDS (SEQ ID NO: 7); and a light chain CDR (LCDR) 1 comprising QDISSF (SEQ ID NO: 29) or QDINSF (SEQ ID NO: 9), an LCDR2 comprising YTS (SEQ ID NO: 10), and optionally an LCDR3 comprising QQGNTLPRT (SEQ ID NO: 11).

In another aspect, the present disclosure provides isolated TfR-binding proteins comprising (i) the antibody or antigen-binding fragment herein and (ii) a cargo linked thereto. In some embodiments, the cargo is a therapeutic compound (e.g., a protein such as an enzyme, e.g., a lysosomal enzyme). In certain embodiments, the enzyme is acid alpha-glucosidase (GAA).

Also provided are pharmaceutical compositions comprising the present antibodies, antigen-binding fragments, or TfR-binding proteins; and pharmaceutically acceptable excipients.

In another aspect, the present disclosure provides nucleic acids and expression vectors encoding the present antibodies, antigen-binding fragments, and TfR-binding proteins, host cells containing such nucleic acids or expression vectors, and methods of using the host cells to produce the antibodies, antigen-binding fragments, and TfR-binding proteins herein. In some embodiments, the method of production comprises culturing the host cell under conditions that permit expression of the antibody or antigen-binding fragment or the TfR-binding protein, and isolating the antibody or antigen-binding fragment or the TfR-binding protein from the cell culture.

In another aspect, the present disclosure provides a method of making a therapeutic molecule capable of crossing the BBB of a human subject, comprising linking (e.g., chemically or recombinantly) a therapeutic moiety of the molecule to the antibody or antigen-binding fragment.

In another aspect, the present disclosure provides a method of delivering a therapeutic molecule across the BBB of a subject in need thereof, comprising administering the therapeutic molecule to the subject, wherein the therapeutic molecule is linked to the antibody or antigen-binding fragment herein. Also provided is an antibody or antigen-binding fragment herein for use in delivering a therapeutic molecule across the BBB of a subject in need thereof. Further provided is use of an antibody or antigen-binding fragment herein for delivering a therapeutic molecule across the BBB of a subject in need thereof, or in the manufacture of a medicament for said purpose.

In another aspect, the present disclosure provides a method of treating a subject in need thereof, comprising administering the isolated TfR-binding protein herein comprising an enzyme (e.g., a lysosomal enzyme) wherein the subject is deficient in the enzyme or an activity thereof. Also provided is an isolated TfR-binding protein herein comprising an enzyme (e.g., a lysosomal enzyme) for use in treating a subject deficient in the enzyme or an activity thereof. Also provided is use of an isolated TfR-binding protein herein comprising an enzyme (e.g., a lysosomal enzyme) for treating, or for the manufacture of a medicament for treating, a subject deficient in the enzyme or an activity thereof. In some embodiments, the enzyme is GAA and the subject has Pompe disease.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIGS. 1A and 1B are diagrams showing amino acid residues of human TfR (FIG. 1A) and cynomolgus monkey TfR (FIG. 1B) having atoms within 4 angstrom distance from 531v25 Fab atoms as determined by cryo-electron microscopy.

FIG. 2A is a panel of schematics illustrating three exemplary formats of anti-TfR binding proteins. “mAb”: monoclonal antibody. Fab: Fab fragment of a full antibody. “Fab-FcOL”: a monovalent antibody fragment having a light chain, a full heavy chain, and an Fc polypeptide that dimerizes with the full heavy chain to form a dimeric Fc domain.

FIG. 2B is a panel of schematics illustrating four exemplary formats of anti-TfR binding proteins comprising a GAA cargo. “Fab-LC-GAA”: GAA fused to the C-terminus of the light chain (LC) of an anti-TfR Fab. “Fab-FcOL-LC-GAA”: GAA fused to the C-terminus of the LC of an anti-TfR Fab-FcOL. “Fab-FcOL-GAA”: GAA fused to the C-terminus of the Fc polypeptide of an anti-TfR Fab-FcOL. “mAb-GAA”: GAA fused to the C-terminus of one of the two heavy chains (HCs) of an anti-TfR mAb.

FIG. 3A is an alignment of two exemplary humanized 531 heavy chains (531 v1 VII and 531 v2 VII) with their corresponding human germline sequences IGHV3-23*05 and IGHJ6-01. HCDR1-3 are underlined. The variation in HCDR1 is in boldface.

FIG. 3B is an alignment of three additional exemplary humanized 531 heavy chains (531 v3 V_{H}, 531 v4 V_{H}, and 531 v5 V_{H}) with their corresponding human germline sequences IGHV1-46*01 and IGHJ6-01. HCDR1-3 are underlined. The variations in HCDR1 are in boldface.

FIG. 3C is an alignment of four exemplary humanized 531 light chains (531 v1 VL, 531 v2 VL, 531 v3 VL, and 531 v4 VL) with their corresponding human germline sequences IGKV1-39*01 and IGKJ4-01. LCDR1-3 are underlined. The variation in LCDR1 is in boldface.

FIGS. 4A-G are graphs showing glycogen levels (mean+SD) in brain (FIG. 4A), spinal cord (FIG. 4B), heart (FIG. 4C), diaphragm (FIG. 4D), gastrocnemius (FIG. 4E), quadriceps (FIG. 4F), and triceps (FIG. 4G) of experimental animals receiving various 531v25-GAA constructs. Asterisk: males and females collectively. “f”: females (significant only). “m”: males (significant only). Dark (top) asterisk: p<0.05 vs. vehicle. Light (bottom) asterisk: p<0.05 vs. neoGAA. “GAA”: recombinant GAA without a carrier.

FIG. 5 is a list of amino acid sequences for the three subunits of the 531v25 Fab-FcOL-GAA molecule. The protein is of human IgG1 isotype subtype. In the light chain (Fab-FcOL-GAA Light Chain), LCDR1-3 are in boldface and the constant region is underlined. In the full heavy chain (Fab-FcOL-GAA Heavy chain_HOLE), HCDR1-3 are in boldface; the constant region is underlined; the NNAS (SEQ ID NO: 53) mutations are double underlined (S301N/T302A/Y303S linear positions, corresponding to S298N/T299A/Y300S Eu numbers); the hole mutations are italicized and in boldface (Y352C/T369S/L371A/Y410V linear positions, corresponding to Y349C/T366S/L368A/Y407V Eu numbers); and the RF mutations are in boldface and double underlined (H438R/Y439F linear positions, corresponding to H435R/Y436F Eu numbers). In the Fc polypeptide/GAA fusion (Fab-FcOL-GAA FCOL_KNOB+GAA), the Fc polypeptide portion is underlined, the GGGG (SEQ ID NO: 42) linker is heavy underlined; the NNAS mutations are double underlined (S78N/T79A/Y80S linear positions, corresponding to S298N/T299A/Y300S Eu numbers); and the knob mutations are italicized and in boldface (S134C/T146W linear positions, corresponding to S354C/T366W Eu numbers). Linear positions refer to the actual positions in the sequences shown.

FIG. 6 is a set of graphs showing tissue specific knockdown of MALAT1 mRNA in brain, heart, gastrocnemius, quadriceps, spleen, and sciatic nerve of experimental animals receiving anti-hTfR-MALAT1-ASO conjugate (“anti-hTfR-ASO”) compared to free MALAT1-ASO (“ASO”) or vehicle (saline). *=p<0.05, **=p<0.005, ***=p<0.0001, vs. vehicle.

### DETAILED DESCRIPTION OF THE INVENTION

The present disclosure provides isolated binding proteins, such as antibodies and antigen-binding fragments thereof, that bind human TfR (hTfR). These TfR-binding proteins bind to an epitope on the extracellular region of hTfR and do not interfere with the interaction between hTfR and transferrin, its natural ligand. The TfR-binding proteins are superior BBB transporters and have improved transcytosis efficiency. Further, the present TfR-binding proteins cross-react with cynomolgus monkey TfR (cTfR), thus allowing pre-clinical studies of the proteins in non-human primates (NHP).

The present disclosure provides also a particular use of the present BBB transporters—to transport an enzyme, e.g., a lysosomal enzyme such as acid alpha-glucosidase (GAA), into the central nervous system (CNS). The present GAA-loaded transporters can be used to restore CNS (e.g., brain and spinal cord) and muscle glycogen to normal levels in patients in need thereof (e.g., Pompe disease patients). The present GAA-loaded transporters will show superior glycogen clearance in the CNS as compared to recombinant GAA used in conventional enzyme replacement therapy.

The present BBB transporters may also be used to transport an oligonucleotide, such as an antisense oligonucleotide or siRNA, into the central nervous system (CNS). Such oligonucleotide-loaded transporters can be used, e.g., to knock down specific mRNA targets in the CNS for therapeutic purposes.

### I. TR-Binding Proteins

The present disclosure provides hTfR-binding proteins, including anti-hTfR antibodies and antigen-binding fragments thereof (also collectively termed “BBB transporters” herein), as well as hTfR-binding proteins comprising such antibodies or antigen-binding fragments and cargos to be transported across the BBB.

### A. TfR-Binding Properties

Human TfR is a homodimer composed of two disulfide-bonded subunits. An exemplary human TfR amino acid sequence may be found at UniProt Accession No. P02786 and at NCBI Accession No. NP001121620.1 and has the following amino acid sequence:

| (SEQ ID NO: 1) | |
|---|---|
| 1 | MMDQARSAFS NLFGGEPLSY TRFSLARQVD GDNSHVEMKL AVDEEENADN |
| 51 | NTKANVTKPK RCSGSICYGT IAVIVFFLIG FMIGYLGYCK GVEPKTECER |
| 101 | LAGTESPVRE EPGEDFPAAR RLYWDDLKRK LSEKLDSTDF TGTIKLLNEN |
| 151 | SYVPREAGSQ KDENLALYVE NQFREFKLSK VWRDQHFVKI QVKDSAQNSV |
| 201 | IIVDKNGRLV YLVENPGGYV AYSKAATVTG KLVHANFGTK KDFEDLYTPV |
| 251 | NGSIVIVRAG KITFAEKVAN AESLNAIGVL IYMDQTKEPI VNAELSFFGH |
| 301 | AHLGTGDPYT PGFPSFNHTQ FPPSRSSGLP NIPVQTISRA AAEKLFGNME |
| 351 | GDCPSDWKTD STCRMVTSES KNVKLTVSNV LKEIKILNIF GVIKGFVEPD |
| 401 | HYVVVGAQRD AWGPGAAKSG VGTALLLKLA QMFSDMVLKD GFQPSRSIIF |
| 451 | ASWSAGDFGS VGATEWLEGY LSSLHLKAFT YINLDKAVLG TSNFKVSASP |
| 501 | LLYTLIEKTM QNVKHPVTGQ FLYQDSNWAS KVEKLTLDNA AFPFLAYSGI |
| 551 | PAVSFCFCED TDYPYLGTTM DTYKELIERI PELNKVARAA AEVAGQFVIK |
| 601 | LTHDVELNLD YERYNSQLLS FVRDLNQYRA DIKEMGLSLQ WLYSARGDFF |
| 651 | RATSRLTTDF GNAEKTDRFV MKKLNDRVMR VEYHFLSPYV SPKESPFRHV |
| 701 | FWGSGSHTLP ALLENLKLRK QNNGAFNETL FRNQLALATW TIQGAANALS |
| 751 | GDVWDIDNEF |

Amino acid residues 1-61 are cytoplasmic. Amino acid residues 62-89 are transmembrane. Amino acid residues 90-760 are extracellular. The extracellular portion (ectodomain) has three domains: the helical domain (residues 606-760), the protease-like domain (residues 121-183, 384-605), and the apical domain (residues 184-383) domain (Sjöström, D., Linnaeus University Dissertations, No. 406/2021; Lawrence et al., Science (1999) 286(5440):779-82).

The present antibody or antigen-binding fragment binds to an epitope located in an area of the protease-like domain (residues 384-605) of hTfR that corresponds to the lateral region of the receptor. This represents a unique epitope that is distinct from known anti-TfR antibodies, which bind to the apical domain of hTfR (see, e.g., antibodies 3 and 3N disclosed in EP3088518A1, which have been shown in WO 2022/174114 to bind to the apical part of hTfR). The present TfR-binding proteins do not compete with transferrin for binding to TfR.

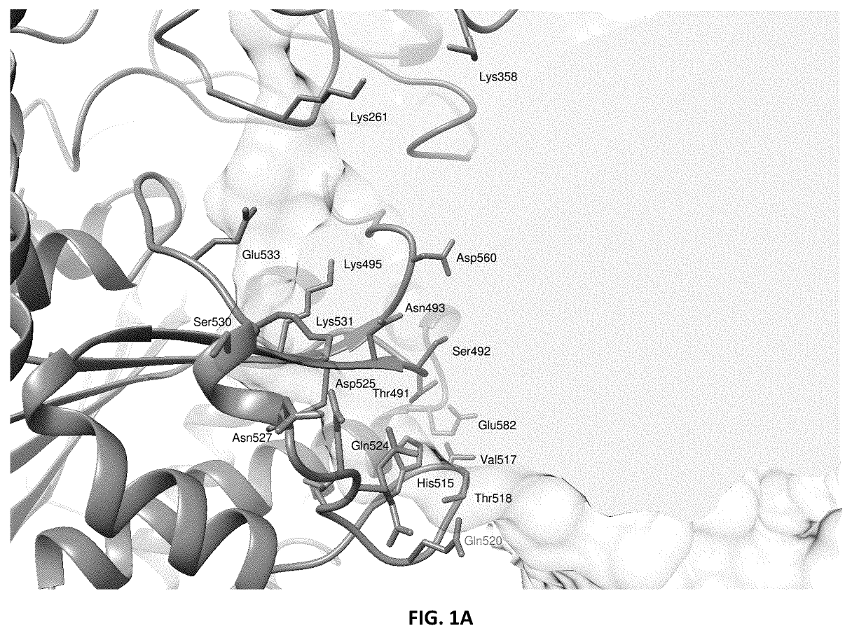

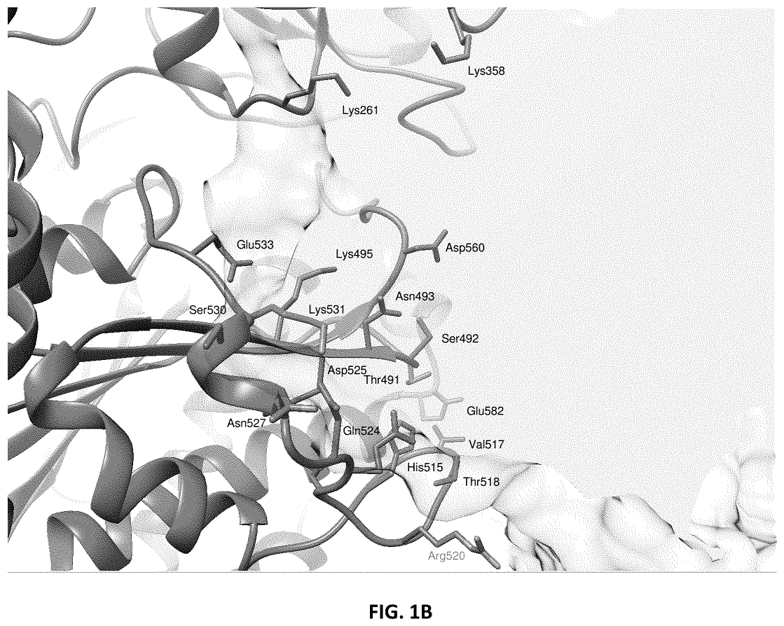

In some embodiments, the present TfR-binding proteins bind to at least one (e.g., 2, 3, 4, 5, 6, 7, 8, 9, 10, 11, 12, 13, 14, 15, 16, or 17) or all of K261, K358, T491, S492, N493, K495, H515, V517, T518, Q520, Q524, D525, N527, S530, K531, E533, D560, and E582 of hTfR (SEQ ID NO: 1) (see FIG. 1A). In certain embodiments, UCSF Chimera software (available at the University of California, San Francisco server) is used for the visualization of the paratope/epitope structure and the determination of the distance in Å and interactions between paratope residues and epitope residues. The term “paratope” refers to residues of an antibody involved in recognition of and binding to the epitope of an antigen. In some embodiments, paratope/epitope pairs may be characterized by, e.g., distance between antibody and antigen atoms in the bound antibody/antigen complex (such as within 4 Å or 5 Å). In some embodiments, paratope/epitope pairs may be characterized by participation in a hydrogen bond interaction, and/or a salt bridge interaction, between antibody and antigen residues.

In some embodiments, the present TfR-binding proteins bind to at least one (e.g., 2, 3, 4, 5, 6, 7, 8, 9, 10, 11, 12, 13, 14, 15, 16, 17, 18, or 19) or all of K261, K358, T491, S492, N493, F494, K495, H515, V517, T518, Q520, L522, Q524, D525, N527, S530, K531, E533, D560, and E582 of hTfR. In certain embodiments, PISA software (available at the European Bioinformatics Institute server) and/or UCSF ChimeraX (available at the University of California, San Francisco server) are used for the visualization of the paratope/epitope structure and the determination of the distance in Å and interactions between paratope residues and epitope residues.

In some embodiments, the present TfR-binding proteins bind to at least one (e.g., 2, 3, 4, 5, 6, 7, 8, 9, 10, 11, 12, 13, 14, 15, 16, 17, 18, 19, 20, 21, 22, 23, 24, 25, or 26) or all of K261, K287, K358, T491, S492, N493, F494, K495, M510, H515, V517, T518, Q520, F521, L522, Y523, Q524, D525, N527, S530, K531, V532, E533, E559, D560, D562 and E582 of hTfR. In certain embodiments, UCSF Chimera software is used for the visualization of the paratope/epitope structure and the determination of the distance in Å and interactions between paratope residues and epitope residues.

In some embodiments, the present TfR-binding proteins bind to an epitope residing in whole or in part in a region spanning T491 to D562 of hTfR, and thus bind to one or more residues in this region.

In some embodiments, the present TfR-binding proteins also bind to the TfR of a non-human primate (NHP), such as macaque (e.g., Macaca fascicularis, aka. cynomolgus monkey). An exemplary amino acid sequence of cynomolgus monkey TfR (cTfR) may be found at NCBI Accession No. XP_045243212.1 and is shown below:

| (SEQ ID NO: 2) | |
|---|---|
| 1 | MMDQARSAFS NLFGGEPLSY TRFSLARQVD GDNSHVEMKL GVDEEENTDN |
| 51 | NTKANGTKPK RCGGNICYGT IAVIIFFLIG FMIGYLGYCK GVEPKTECER |
| 101 | LAGTESPARE EPEEDFPAAP RLYWDDLKRK LSEKLDTTDF TSTIKLLNEN |
| 151 | LYVPREAGSQ KDENLALYIE NQFREFKLSK VWRDQHFVKI QVKDSAQNSV |
| 201 | IIVDKNGGLV YLVENPGGYV AYSKAATVTG KLVHANFGTK KDFEDLDSPV |
| 251 | NGSIVIVRAG KITFAEKVAN AESLNAIGVL IYMDQTKFPI VKADLSFFGH |
| 301 | AHLGTGDPYT PGFPSFNHTQ FPPSQSSGLP NIPVQTISRA AAEKLFGNME |
| 351 | GDCPSDWKTD STCKMVTSEN KSVKLTVSNV LKETKILNIF GVIKGFVEPD |
| 401 | HYVVVGAQRD AWGPGAAKSS VGTALLLKLA QMFSDMVLKD GFQPSRSIIF |
| 451 | ASWSAGDFGS VGATEWLEGY LSSLHLKAFT YINLDKAVLG TSNFKVSASP |
| 501 | LLYTLIEKTM QDVKHPVTGR SLYQDSNWAS KVEKLTLDNA AFPFLAYSGI |
| 551 | PAVSFCFCED TDYPYLGTTM DTYKELVERI PELNKVARAA AEVAGQFVIK |
| 601 | LTHDTELNLD YERYNSQLLL FLRDLNQYRA DVKEMGLSLQ WLYSARGDEF |
| 651 | RATSRLTTDF RNAEKRDKFV MKKLNDRVMR VEYYFLSPYV SPKESPFRHV |
| 701 | FWGSGSHTLS ALLESLKLRR QNNSAFNETL FRNQLALATW TIQGAANALS |
| 751 | GDVWDIDNEF |

In some embodiments, the present TfR-binding proteins bind to at least one (e.g., 2, 3, 4, 5, 6, 7, 8, 9, 10, 11, 12, 13, 14, 15, 16, or 17) or all of K261, K358, T491, S492, N493, K495, H515, V517, T518, R520, Q524, D525, N527, S530, K531, E533, D560 and E582 of cTfR (see FIG. 1B). These residues are identical between hTfR and cTfR, except for the Q520R substitution from hTfR to cTfR. In certain embodiments, UCSF Chimera software is used for the visualization of the paratope/epitope structure and the determination of the distance in Å and interactions between paratope residues and epitope residues.

In some embodiments, the present TfR-binding proteins bind to at least one (e.g., 2, 3, 4, 5, 6, 7, 8, 9, 10, 11, 12, 13, 14, 15, 16, or 17) or all of K261, K358, T491, S492, N493, F494, K495, H515, V517, T518, R520, L522, Q524, D525, N527, S530, K531, E533, D560 and E582 of cTfR (see FIG. 1B). These residues are identical between hTfR and cTfR, except for the Q520R substitution from hTfR to cTfR. In certain embodiments, PISA and/or UCSF ChimeraX software are used for the visualization of the paratope/epitope structure and the determination of the distance in Å and interactions between paratope residues and epitope residues.

In some embodiments, the present TfR-binding proteins bind to at least one (e.g., 2, 3, 4, 5, 6, 7, 8, 9, 10, 11, 12, 13, 14, 15, 16, 17, 18, 19, 20, 21, 22, 23, 24, 25, or 26) or all of K261, K287, K358, T491, S492, N493, F494, K495, M510, H515, V517, T518, R520, S521, L522, Y523, Q524, D525, N527, S530, K531, V532, E533, E559, D560, D562 and E582 of cTfR. These residues are identical between hTfR and cTfR except for the Q520R and F521S substitutions from hTfR to cTfR. In certain embodiments, UCSF Chimera software is used for the visualization of the paratope/epitope structure and the determination of the distance in Å and interactions between paratope residues and epitope residues.

Residues of hTfR and/or cTfR bound by the present TfR-binding proteins may be identified using, e.g., UCSF Chimera, PISA, or UCSF ChimeraX software, or any combination thereof.

In some embodiments, the present TfR-binding proteins bind to an epitope residing in whole or in part in a region spanning T491 to D562 of cTfR, and thus bind to one or more residues in this region.

The ectodomains (residues 90-760) of hTfR and cTfR are 95% identical and 97% homologous to each other. An alignment between the two ectodomains is shown below. In the alignment, a “*” (asterisk) indicates positions which have a single, fully conserved residue; a “:” (colon) indicates conservation between groups of strongly similar properties (scoring >0.5 in the Gonnet PAM 250 matrix), and a “.” (period) indicates conservation between groups of weakly similar properties (scoring≤ 0.5 in the Gonnet PAM 250 matrix). Regions forming the protease-like domain (Sjöström, supra) are underlined.

| | |
|---|---|
| hTfR | GVEPKTECERLAGTESPVREEPGEDFPAARRLYWDDLKRKLSEKLDSTDFTGTIKLLNEN |
| cTfR | GVEPKTECERLAGTESPAREEPEEDFPAAPRLYWDDLKRKLSEKLDTTDFTSTIKLLNEN |
| | *****************.**** ****** ****************:****.******** |
| hTfR | SYVPREAGSQKDENLALYVENQFREFKLSKVWRDQHFVKIQVKDSAQNSVIIVDKNGRLV |
| cTfR | LYVPREAGSQKDENLALYIENQFREFKLSKVWRDQHFVKIQVKDSAQNSVIIVDKNGGLV |
| | *****************:************************************** ** |
| hTfR | 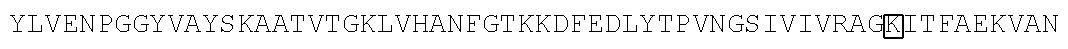 |
| cTfR | 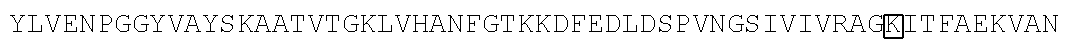 |
| | 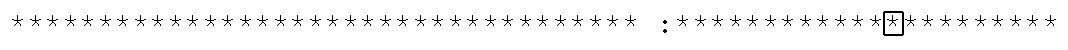 |
| hTfR | AESLNAIGVLIYMDQTKFPIVNAELSFFGHAHLGTGDPYTPGFPSFNHTQFPPSRSSGLP |
| cTfR | AESLNAIGVLIYMDQTKFPIVKADLSFFGHAHLGTGDPYTPGFPSFNHTQFPPSQSSGLP |
| | *********************:*:******************************:***** |
| hTfR | 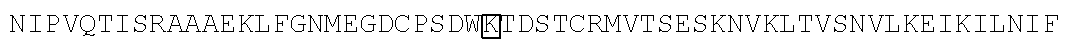 |
| cTfR | 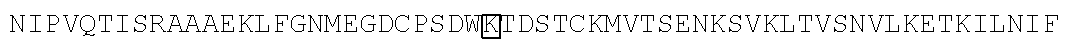 |
| | 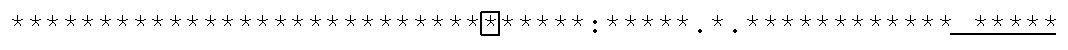 |
| hTfR | GVIKGFVEPDHYVVVGAQRDAWGPGAAKSGVGTALLLKLAQMFSDMVLKDGFQPSRSIIF |
| cTfR | GVIKGFVEPDHYVVVGAQRDAWGPGAAKSSVGTALLLKLAQMFSDMVLKDGFQPSRSIIF |
| | *****************************.****************************** |
| hTfR | 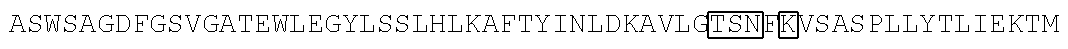 |
| cTfR | 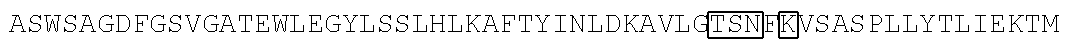 |
| | 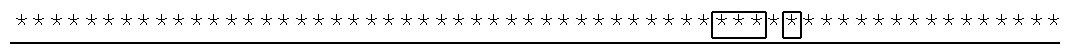 |
| hTfR | 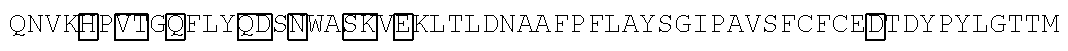 |
| cTfR | 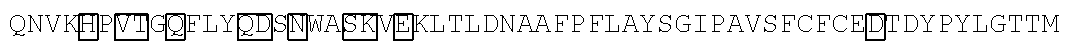 |
| | 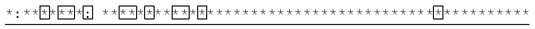 |
| hTfR | 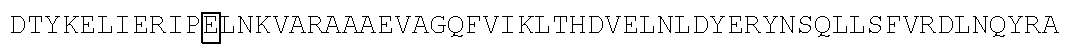 |
| cTfR | 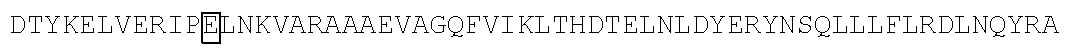 |
| | 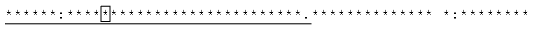 |
| hTfR | DIKEMGLSLOWLYSARGDFFRATSRLTTDFGNAEKTDRFVMKKLNDRVMRVEYHFLSPYV |
| cTfR | DVKEMGLSLOWLYSARGDFFRATSRLTTDFRNAEKRDKFVMKKLNDRVMRVEYYFLSPYV |
| | *:**************************** **** *:***************:****** |
| hTfR | SPKESPFRHVFWGSGSHTLPALLENLKLRKQNNGAFNETLERNQLALATWTIQGAANALS |
| cTfR | SPKESPFRHVFWGSGSHTLSALLESLKLRRQNNSAFNETLFRNQLALATWTIQGAANALS |
| | ******************* ****.****:***.************************** |
| hTfR | GDVWDIDNEF (residues 90-760 of SEQ ID NO: 1) |
| cTfR | GDVWDIDNEF (residues 90-760 of SEQ ID NO: 2) |
| | ********** |

In some embodiments, the present TfR-binding proteins bind to residues that are conserved between hTfR and cTfR ectodomains. In further embodiments, the present TfR-binding proteins bind to residues that are conserved between the protease-like domains of hTfR and cTfR ectodomains. In further embodiments, the present TfR-binding proteins bind to one or more residues in a region spanning position 384-605 (e.g., one or more residues in a region spanning positions 491-562) of hTfR, for example, residues conserved between hTfR and cTfR in this region. In some embodiments, the present TfR-binding proteins bind at least one or all of the residues boxed in the above alignment.

The present anti-TfR antibodies and antigen-binding fragments thereof bind specifically to hTfR and cTfR. By “specifically” is meant that the antibodies and fragments bind to hTfR and cTfR with an affinity described herein or higher. For serving as carriers to cross the BBB, the BBB transporters may have a suitable affinity for hTfR. In addition, to facilitate pre-clinical studies of the BBB transporters in NHP animal models, the BBB transporters may have a suitable affinity for cTfR, and the differential (ratio) between the BBB transporters' affinity for cTfR and their affinity for hTfR may be within about 1 log. Several techniques can be used to characterize TfR-binding affinity (K_{D}), such as surface plasmon resonance (SPR, using, e.g., BIAcore™) or bio-layer interferometry (BLI, using, e.g., Octet™ from ForteBio). Flow cytometry assay (e.g., FACS) using cells expressing membrane-bound hTfR or cTfR can also be used to determine EC₅₀ or IC₅₀ values of the BBB transporters; these values are indicative of the binding to human and cynomolgus TfR in their native conformation.

In some embodiments, the BBB transporters have a Kp of about 1-50 nM (e.g., 1-30, 1-20, or 1-10 nM) for hTfR and have a K_{D} of about 1-200 nM (e.g., 1-150 or 1-100 nM) for cTfR as determined by SPR (e.g., using BIAcore™). In some embodiments, the SPR assay is performed with the ectodomain of the TfR to be evaluated. Such an assay is illustrated in Example 2B below. In some embodiments, the ratio of the BBB transporters' binding affinity for hTfR versus cTfR is between 1:1 and 1:20, between 1:1 and 1:15, between 1:1 and 1:10, between 1:1 and 1:9, between 1:2 and 1:9, between 1:3 and 1:9, between 1:4 and 1:9, between 1:5 and 1:9, or between 1:6 and 1:9 (e.g., about 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20).

In some embodiments, the BBB transporters have a K_{D} of about 1-50 nM (e.g., 1-30, 1-20, or 1-10 nM) for hTfR and have a Kp of about 1-200 nM (e.g., 1-150 or 1-100 nM) for cTfR as determined by BLI (e.g., using Octet®). In some embodiments, the BLI is performed with the ectodomain of the TfR to be evaluated. Such an assay is further illustrated in Example 3A below. In some embodiments, the ratio of the BBB transporters' binding affinity for hTfR versus cTfR is between 1:1 and 1:20, between 1:1 and 1:15, between 1:1 and 1:10, between 1:1 and 1:9, between 1:2 and 1:9, between 1:3 and 1:9, between 1:4 and 1:9, between 1:5 and 1:9, or between 1:6 and 1:9 (e.g., about 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20).

In some embodiments, the BBB transporters have an EC₅₀ of about 1-50 nM (e.g., 1-30, 1-20, or 1-10 nM) for hTfR and have an EC₅₀ of about 1-200 nM (e.g., 1-150 or 1-100 nM) for cTfR as determined by flow cytometry (e.g., FACS) with mammalian cells engineered to express the TfR to be evaluated. In some embodiments, the mammalian cells may be murine cells (e.g., murine pre-B cells), hamster cells, or human cells. Such an assay is further illustrated in Example 5A below. In some embodiments, the ratio of the BBB transporters' EC50 for hTfR versus cTfR is between 1:1 and 1:20, between 1:1 and 1:15, between 1:1 and 1:10, between 1:1 and 1:9, between 1:2 and 1:9, between 1:3 and 1:9, between 1:4 and 1:9, between 1:5 and 1:9, or between 1:6 and 1:9 (e.g., about 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20).

BBB transporters with any combination of the above functional properties are also contemplated.

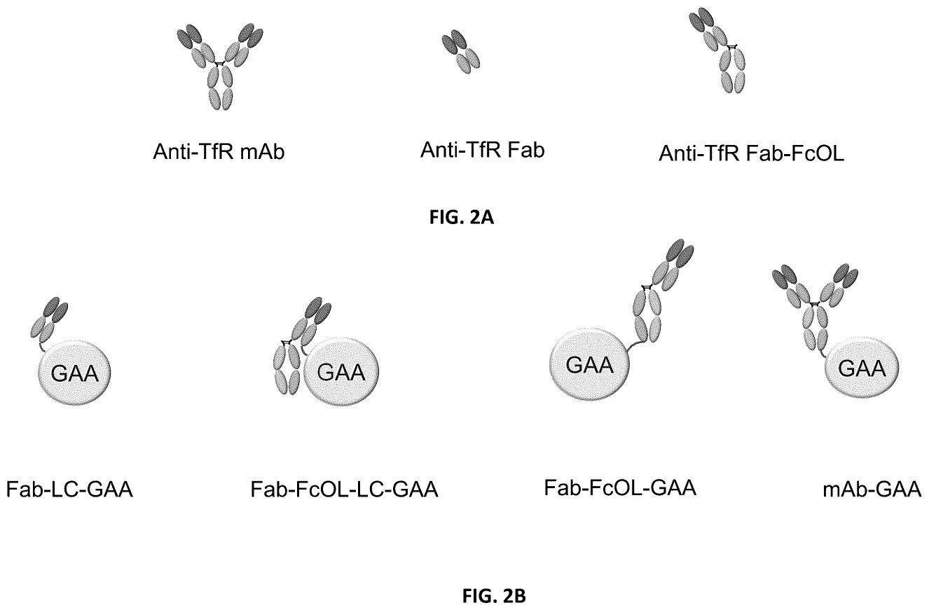

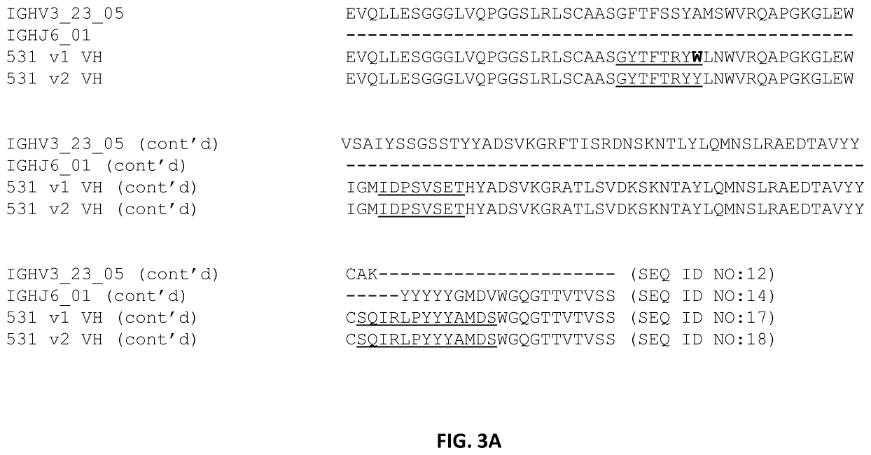

Exemplary formats of the TfR-binding proteins are illustrated in FIG. 2A (antibodies and fragments thereof) and FIG. 2B (cargo-loaded TfR-binding proteins). The formats will be further described below.

### B. Anti-TfR Antibodies and Antigen-Binding Fragments

The TfR-binding proteins herein include chimeric or humanized anti-TfR antibodies and antigen-binding fragments with murine-originated antigen-binding domains. These antibodies and antigen-binding fragments can be used as transporters to carry cargos (payloads) across the BBB.

The term “antibody” (Ab) or “immunoglobulin” (Ig) refers to a tetramer comprising two heavy (H) chains and two light (L) chains interconnected by disulfide bonds. Each heavy chain is comprised of a heavy chain variable region or domain (V_{H}) and a heavy chain constant region (C_{H}). Each light chain is composed of a light chain variable region or domain (V_{L}) and a light chain constant region (CL). The V_{H} and V_{L} domains can be subdivided further into regions of hypervariability, termed “complementarity-determining regions” (CDRs), interspersed with regions that are more conserved, termed “framework regions” (FRs). Each V_{H} and V_{L} is composed of three CDRs (HCDR herein designates a CDR from the heavy chain; and LCDR herein designates a CDR from the light chain) and four FRs, arranged from amino-terminus to carboxyl-terminus in the following order: FR1, CDR1, FR2, CDR2, FR3, CDR3, and FR4.

The precise amino acid sequence boundaries of a given CDR or FR can be defined by several well-known systems, including those described by Kabat et al., 5th Ed., Public Health Service, National Institutes of Health, Bethesda, MD (1991) (“Kabat” system); Al-Lazikani et al., J Mol Biol. (1997) 273:927-48) (“Chothia” system); MacCallum et al., J Mol Biol. (1996) 262:732-45 (“contact” system); Lefranc et al., Dev Comp Immunol. (2003) 27(1): 55-77 (“IMGT” system); Honegger and Plückthun, J Mol Biol. (2001) 309(3):657-70 (“Aho” system); and Whitelegg and Rees, Protein Eng. (2000) 13(12):819-24 (“AbM” system). The boundaries of a given CDR or FR may vary depending on the system used. For example, the Kabat system is based on sequence alignments, while the Chothia system is based on structural information. Numbering for both the Kabat and Chothia systems is based upon the most common antibody region sequence lengths, with insertions accommodated by insertion letters, for example, “30a.” The two systems place certain insertions and deletions (“indels”) at different positions, resulting in differential numbering. The contact system is based on analysis of complex crystal structures and is similar in many respects to the Chothia system. In certain embodiments, the CDRs of the antibodies described herein can be defined by a system selected from Kabat, Chothia, IMGT, Aho, AbM, or combinations thereof. Unless otherwise specified, the CDRs herein are defined by the IMGT system.

In some embodiments, the BBB transporters herein have a configuration that differs from a full antibody having two full-length heavy chains and two full-length light chains. For example, the BBB transporters are antigen-binding fragments of a full, tetrameric antibody and yet retain the TfR-binding properties of the full antibody. The term “antigen-binding fragment” or “antigen-binding portion” herein encompasses genetically engineered and/or otherwise modified forms of immunoglobulins that do not have the conventional full-length tetrameric structure. The term encompasses intrabodies, peptibodies, diabodies, triabodies, tetrabodies, Fv, Fab, Fab′, Fab′-SH, F(ab′)2, single-chain antibody molecules (e.g., scFv or sFv), tandem di-scFv, and tandem tri-scFv. Exemplary anti-TfR antigen-binding fragments include the Fab fragments and Fab-FcOL fragments described herein.

In some embodiments, the anti-TfR antibodies herein are humanized antibodies. A “humanized” antibody is an antibody in which all or substantially all CDR amino acid residues are derived from non-human CDRs (e.g., mouse) and all or substantially all FR amino acid residues are derived from human FRs (i.e., acceptor). A humanized antibody may also include at least a portion of an antibody heavy and/or light chain constant region derived from a human antibody. Compared to the non-human parental antibody from which a humanized antibody is derived, a humanized antibody has reduced immunogenicity to humans. To retain the specificity and affinity of the parental antibody, some FR residues in the human acceptor may be substituted with corresponding residues from the non-human parental antibody (back mutations).

### 1. TfR-Binding Domains

The TfR-binding domains of the BBB transporters (including anti-TfR antibodies or antigen-binding fragments thereof) may comprise antibody heavy chain variable domain (V_{H}) and antibody light chain variable domain (V_{L}) in which the heavy chain CDR (HCDR) 1-3 and the light chain CDR (LCDR) 1 and 2 are derived from a murine parental anti-TfR antibody. In certain embodiments, the HCDR1-3 and the LCDR1-3 are derived from a murine parental anti-TfR antibody.

In some embodiments, the BBB transporters herein comprise an HCDR1 comprising X₁YTFTRYX₂, optionally wherein X₁ may be G or D, and X2 may be W or Y (SEQ ID NO: 50). For example, the HCDR1 may be GYTFTRYY (SEQ ID NO: 26), or GYTFTRYW (SEQ ID NO: 27), or DYTFTRYW (SEQ ID NO: 5).

In some embodiments, the BBB transporters herein comprise an HCDR2 comprising IDPSVSEX₃, optionally wherein X₃ may be T or C (SEQ ID NO: 51). For example, the HCDR2 may comprise IDPSVSET (SEQ ID NO: 28) or IDPSVSEC (SEQ ID NO: 6).

In some embodiments, the BBB transporters herein comprise an HCDR3 comprising SQIRLPYYYAMDS (SEQ ID NO: 7).

In some embodiments, the BBB transporters herein comprise an LCDR1 comprising QDIX₄SF, optionally wherein X₄ may be S or N (SEQ ID NO: 52). For example, the LCDR1 may comprise QDISSF (SEQ ID NO: 29) or QDINSF (SEQ ID NO: 9).

In some embodiments, the BBB transporters herein comprise an LCDR2 comprising YTS (SEQ ID NO: 10).

In some embodiments, the BBB transporters herein comprise an LCDR3 comprising QQGNTLPRT (SEQ ID NO: 11).

In some embodiments, the BBB transporters comprise a) HCDR1-3 and b) LCDR1 and 2 or LCDR1-3, as described in the above paragraphs. That is, the HCDR1-3 comprise SEQ ID NOs: 26, 28, and 7, respectively; SEQ ID NOs: 27, 28, and 7, respectively; or SEQ ID NOs: 5, 6, and 7, respectively; and/or i) the LCDR 1 and 2 comprise SEQ ID NOs: 29 and 10, respectively, or SEQ ID NOs: 9 and 10, respectively; or ii) the LCDR1-3 comprise SEQ ID NOs: 29, 10, and 11, respectively; or SEQ ID NOs: 9, 10, and 11, respectively. In certain embodiments, the BBB transporters comprise (i) HCDR1-3 comprising SEQ ID NOs: 26, 28, and 7, respectively, and LCDR1 and 2 comprising SEQ ID NOs: 29 and 10, respectively, or (ii) HCDR1-3 comprising SEQ ID NOs: 5-7, respectively, and LCDR1 and 2 comprising SEQ ID NOs: 9 and 10, respectively. In certain embodiments, the BBB transporters comprise (i) HCDR1-3 comprising SEQ ID NOs: 26, 28, and 7, respectively, and LCDR1-3 comprising SEQ ID NOs: 29, 10, and 11, respectively, or (ii) HCDR1-3 comprising SEQ ID NOs: 5-7, respectively, and LCDR1-3 comprising SEQ ID NOs: 9-11, respectively.

In some embodiments, the BBB transporters comprise HCDR1-3, LCDR1, and LCDR2 as described in the above paragraphs, and further comprise a heavy chain framework (HFR) 1 whose last residue is serine (S), a light chain framework (LFR) 3 whose first residue is arginine (R), or both. For example, in certain embodiments, the BBB transporters comprise
an HCDR1 comprising GYTFTRYY (SEQ ID NO: 26), GYTFTRYW (SEQ ID NO: 27), or DYTFTRYW (SEQ ID NO: 5);
an HCDR2 comprising IDPSVSET (SEQ ID NO: 28) or IDPSVSEC (SEQ ID NO: 6); and
an HCDR3 comprising SQIRLPYYYAMDS (SEQ ID NO: 7); and
an LCDR1 comprising QDISSF (SEQ ID NO: 29) or QDINSF (SEQ ID NO: 9),
an LCDR2 comprising YTS (SEQ ID NO: 10), and
optionally an LCDR3 comprising QQGNTLPRT (SEQ ID NO: 11), and further comprise an HFR1 whose last residue is S and/or an LFR3 whose first residue is R (e.g., an HFR1 whose last residue is S and an LFR3 whose first residue is R).
   In certain embodiments, the BBB transporters comprise
an HCDR1 comprising GYTFTRYY (SEQ ID NO: 26), GYTFTRYW (SEQ ID NO: 27), or DYTFTRYW (SEQ ID NO: 5);
an HCDR2 comprising IDPSVSET (SEQ ID NO: 28) or IDPSVSEC (SEQ ID NO: 6); and
an HCDR3 comprising SQIRLPYYYAMDS (SEQ ID NO: 7); and
an LCDR1 comprising QDISSF (SEQ ID NO: 29) or QDINSF (SEQ ID NO: 9),
an LCDR2 comprising YTS (SEQ ID NO: 10), and
an LCDR3 comprising QQGNTLPRT (SEQ ID NO: 11), and further comprise an HFR1 whose last residue is S and/or an LFR3 whose first residue is R (e.g., an HFR1 whose last residue is S and an LFR3 whose first residue is R).

In some embodiments, the HCDR1-3 comprise SEQ ID NOs: 26, 28, and 7, respectively; SEQ ID NOs: 27, 28, and 7, respectively; or SEQ ID NOs: 5, 6, and 7, respectively; the LCDR1 and 2 comprise SEQ ID NOs: 29 and 10, respectively; or SEQ ID NOs: 9 and 10, respectively; and the HFR1 has a last residue of S and/or the LFR3 has a first residue of R. In particular embodiments, the BBB transporters comprise HCDR1-3 comprising SEQ ID NOs: 26, 28, and 7, respectively; LCDR1 and 2 comprising SEQ ID NOs: 29 and 10, respectively; and an HFR1 whose last residue is S and/or an LFR3 whose first residue is R (e.g., an HFR1 whose last residue is S and an LFR3 whose first residue is R). In other embodiments, the BBB transporters comprise HCDR1-3 comprising SEQ ID NOs: 5-7, respectively; LCDR1 and 2 comprising SEQ ID NOs: 9 and 10, respectively; and an HFR1 whose last residue is S and/or an LFR3 whose first residue is R (e.g., an HFR1 whose last residue is S and an LFR3 whose first residue is R).

In some embodiments, the BBB transporters comprise HCDR1-3 and LCDR1-3 as described in the above paragraphs, and further comprise an HFR1 whose last residue is S, an LFR3 whose first residue is R, or both. For example, in certain embodiments, the HCDR1-3 comprise SEQ ID NOs: 26, 28, and 7, respectively; SEQ ID NOs: 27, 28, and 7, respectively; or SEQ ID NOs: 5, 6, and 7, respectively; the LCDR1-3 comprise SEQ ID NOs: 29, 10, and 11, respectively; or SEQ ID NOs: 9-11, respectively; and the HFR1 has a last residue of S and/or the LFR3 has a first residue of R. In particular embodiments, the BBB transporters comprise HCDR1-3 comprising SEQ ID NOs: 26, 28, and 7, respectively; LCDR1-3 comprising SEQ ID NOs: 29, 10, and 11, respectively; and an HFR1 whose last residue is S and/or an LFR3 whose first residue is R (e.g., an HFR1 whose last residue is S and an LFR3 whose first residue is R). In other embodiments, the BBB transporters comprise HCDR1-3 comprising SEQ ID NOs: 5-7, respectively; LCDR1-3 comprising SEQ ID NOs: 9-11, respectively; and an HFR1 whose last residue is S and/or an LFR3 whose first residue is R (e.g., an HFR1 whose last residue is S and an LFR3 whose first residue is R).

In some embodiments, the BBB transporters comprise HCDR1-3 of SEQ ID NOs: 26, 28, and 7, respectively, and LCDR1 and 2 of SEQ ID NOs: 29 and 10, respectively, wherein said HCDR1-3 and LCDR1 and 2 comprise one to five (e.g., 1, 2, 3, 4, or 5, one to two, one to three, or one to four) mutations in total across the five CDRs, wherein the mutations do not occur at the following positions:
Y2, R6, Y7, and optionally Y8 of SEQ ID NO: 26 (HCDR1),
D2, S4, V5, and E7 of SEQ ID NO: 28 (HCDR2),
R4, L5, P6, Y7, Y8, and Y9 of SEQ ID NO: 7 (HCDR3),
F6 of SEQ ID NO: 29 (LCDR1), and
Y1 of SEQ ID NO: 10 (LCDR2),
wherein the BBB transporters further comprise an HFR1 whose last residue is S and/or an LFR3 whose first residue is R (e.g., an HFR1 whose last residue is S and an LFR3 whose first residue is R).

In some embodiments, the BBB transporters comprise HCDR1-3 of SEQ ID NOs: 26, 28, and 7, respectively, and LCDR1-3 of SEQ ID NOs: 29, 10, and 11, respectively, wherein said HCDR1-3 and LCDR1-3 comprise one to five (e.g., 1, 2, 3, 4, or 5, one to two, one to three, or one to four) mutations in total across the six CDRs, wherein the mutations do not occur at the following positions:
Y2, R6, Y7, and optionally Y8 of SEQ ID NO: 26 (HCDR1),
D2, S4, V5, and E7 of SEQ ID NO: 28 (HCDR2),
R4, L5, P6, Y7, Y8, and Y9 of SEQ ID NO: 7 (HCDR3),
F6 of SEQ ID NO: 29 (LCDR1), and
Y1 of SEQ ID NO: 10 (LCDR2),
wherein the BBB transporters further comprise an HFR1 whose last residue is S and/or an

LFR3 whose first residue is R (e.g., an HFR1 whose last residue is S and an LFR3 whose first residue is R).

In some embodiments, the BBB transporters, when bound to hTfR, comprise a V_{H} comprising, as defined by IMGT numbering:
optionally S26 that is within 4.0 Å of residue K261 and K358 of SEQ ID NO: 1;
Y28 that is within 4.0 Å of residue K261 of SEQ ID NO: 1;
R36 that is within 4.0 Å of residues K495, K531, and E533 of SEQ ID NO: 1;
Y37 that is within 4.0 Å of residues N493 and D560 of SEQ ID NO: 1;
optionally Y38 that is within 4.0 Å of residue D525 of SEQ ID NO: 1
D57 that is within 4.0 Å of residues N527 and K531 of SEQ ID NO: 1;
S59 that is within 4.0 Å of residues S530 and K531 of SEQ ID NO: 1;
V62 that is within 4.0 Å of residues N527 and S530 of SEQ ID NO: 1;
E64 that is within 4.0 Å of residue N527 of SEQ ID NO: 1;
R108 that is within 4.0 Å of residues N493 and D560 of SEQ ID NO: 1;
L109 that is within 4.0 Å of residues S492 and D560 of SEQ ID NO: 1;
P110 that is within 4.0 Å of residues S492 and N493 of SEQ ID NO: 1;
Y111 that is within 4.0 Å of residues S492, F494, H515, T518, and L522 of SEQ ID NO: 1;
Y112 that is within 4.0 Å of residues T491, S492, H515, V517, and T518 of SEQ ID NO: 1; and
Y113 that is within 4.0 Å of residues Q520 and Q524 of SEQ ID NO: 1; and a VL comprising, as defined by IMGT numbering:
F38 that is within 4.0 Å of residue Q520 of SEQ ID NO: 1;
Y56 that is within 4.0 Å of residues V517 and T518 of SEQ ID NO: 1; and
R66 that is within 4.0 Å of residue E582 of SEQ ID NO: 1.
   In certain embodiments, UCSF Chimera, PISA, or UCSF ChimeraX software, or any combination thereof, is used for the visualization of the paratope/epitope structure and the determination of the distance in Å and interactions between paratope residues and epitope residues.

In some embodiments, the BBB transporters, when bound to hTfR, comprise a V_{H} comprising, as defined by IMGT numbering:
optionally S26 that can form a hydrogen bond with residue K261 of SEQ ID NO: 1;
R36 that can form a hydrogen bond with residue K531 of SEQ ID NO: 1;
Y37 that can form a hydrogen bond with residue N493 of SEQ ID NO: 1;
optionally Y38 that can form a hydrogen bond with residue D525 of SEQ ID NO: 1;
D57 that can form a hydrogen bond with residue N527 of SEQ ID NO: 1 and a salt bridge with residue K531 of SEQ ID NO: 1;
S59 that can form a hydrogen bond with residue K531 of SEQ ID NO: 1;
R108 that can form a hydrogen bond with residue N493 of SEQ ID NO: 1 and a salt bridge with residue D560 of SEQ ID NO: 1;
Y111 that can form a hydrogen bond with residue S492 of SEQ ID NO: 1 and a hydrogen bond with residue L522 of SEQ ID NO: 1; and
Y113 that can form a hydrogen bond with residue Q524 of SEQ ID NO: 1; and a V_{L} comprising, as defined by IMGT numbering:
Y56 that can form a hydrogen bond with residue V517 and a hydrogen bond with residue T518 of SEQ ID NO: 1; and
R66 that can form a salt bridge with residue E582 of SEQ ID NO: 1.

In some embodiments, the BBB transporters comprise HCDR1-3 of SEQ ID NOs: 26, 28, and 7, respectively, LCDR1 and 2 of SEQ ID NOs: 29 and 10, respectively, optionally LCDR3 of SEQ ID NO: 11, and optionally an HFR1 whose last residue is S and/or an LFR3 whose first residue is R (e.g., an HFR1 whose last residue is S and an LFR3 whose first residue is R), wherein the BBB transporters, when bound to hTfR, comprise a V_{H} comprising, as defined by IMGT numbering:
a) S26 that is within 4.0 Å of residue K261 and K358 of SEQ ID NO: 1;
b) Y28 that is within 4.0 Å of residue K261 of SEQ ID NO: 1;
c) R36 that is within 4.0 Å of residues K495, K531, and E533 of SEQ ID NO: 1;
d) Y37 that is within 4.0 Å of residues N493 and D560 of SEQ ID NO: 1;
e) Y38 that is within 4.0 Å of residue D525 of SEQ ID NO: 1
f) D57 that is within 4.0 Å of residues N527 and K531 of SEQ ID NO: 1;
g) S59 that is within 4.0 Å of residues S530 and K531 of SEQ ID NO: 1; V62 that is within 4.0 Å of residues N527 and S530 of SEQ ID NO: 1; h) E64 that is within 4.0 Å of residue N527 of SEQ ID NO: 1; i)
j) R108 that is within 4.0 Å of residues N493 and D560 of SEQ ID NO: 1;
k) L109 that is within 4.0 Å of residues S492 and D560 of SEQ ID NO: 1;
l) P110 that is within 4.0 Å of residues S492 and N493 of SEQ ID NO: 1;
m) Y111 that is within 4.0 Å of residues S492, F494, H515, T518, and L522 of SEQ ID NO: 1;
n) Y112 that is within 4.0 Å of residues T491, S492, H515, V517, and T518 of SEQ ID NO: 1; and/or
o) Y113 that is within 4.0 Å of residues Q520 and Q524 of SEQ ID NO: 1; and/or a V_{L} comprising, as defined by IMGT numbering:
p) F38 that is within 4.0 Å of residue Q520 of SEQ ID NO: 1;
q) Y56 that is within 4.0 Å of residues V517 and T518 of SEQ ID NO: 1; and/or
r) R66 that is within 4.0 Å of residue E582 of SEQ ID NO: 1;
or any combination (e.g., any 1, 2, 3, 4, 5, 6, 7, 8, 9, 10, 11, 12, 13, 14, 15, 16, 17, or all 18) of a)-r). In certain embodiments, UCSF Chimera, PISA, or UCSF ChimeraX software, or any combination thereof is used for the visualization of the paratope/epitope structure and the determination of the distance in Å and interactions between paratope residues and epitope residues.

In some embodiments, the BBB transporters comprise HCDR1-3 of SEQ ID NOs: 26, 28, and 7, respectively, LCDR1 and 2 of SEQ ID NOs: 29 and 10, respectively, optionally LCDR3 of SEQ ID NO: 11, and optionally an HFR1 whose last residue is S and/or an LFR3 whose first residue is R (e.g., an HFR1 whose last residue is S and an LFR3 whose first residue is R), wherein the BBB transporters, when bound to hTfR, comprise a V_{H} comprising, as defined by IMGT numbering:
a) S26 that can form a hydrogen bond with residue K261 of SEQ ID NO: 1;
b) R36 that can form a hydrogen bond with residue K531 of SEQ ID NO: 1;
c) Y37 that can form a hydrogen bond with residue N493 of SEQ ID NO: 1;
d) Y38 that can form a hydrogen bond with residue D525 of SEQ ID NO: 1;
e) D57 that can form a hydrogen bond with residue N527 of SEQ ID NO: 1 and a salt bridge with residue K531 of SEQ ID NO: 1;
f) S59 that can form a hydrogen bond with residue K531 of SEQ ID NO: 1;
g) R108 that can form a hydrogen bond with residue N493 of SEQ ID NO: 1 and a salt bridge with residue D560 of SEQ ID NO: 1;
h) Y111 that can form a hydrogen bond with residue S492 of SEQ ID NO: 1 and a hydrogen bond with residue L522 of SEQ ID NO: 1; and/or
i) Y113 that can form a hydrogen bond with residue Q524 of SEQ ID NO: 1; and/or a V_{L} comprising, as defined by IMGT numbering:
j) Y56 that can form a hydrogen bond with residue V517 and a hydrogen bond with residue T518 of SEQ ID NO: 1; and
k) R66 that can form a salt bridge with residue E582 of SEQ ID NO: 1;
or any combination (e.g., any 1, 2, 3, 4, 5, 6, 7, 8, 9, 10, or all 11) of a)-k).

In some embodiments, the BBB transporters comprise a Vu comprising residues S26, Y28, R36, Y37, Y38, D57, S59, V62, E64, R108, L109, P110, Y111, Y112 and Y113; and a V_{L} comprising residues F38, Y56, and R66; wherein the residue positions are defined according to IMGT numbering. In certain embodiments, said BBB transporters bind to an epitope of hTfR comprising residues K261, K358, T491, S492, N493, F494, K495, H515, V517, T518, Q520, L522, Q524, D525, N527, S530, K531, E533, and D560, and E582 of SEQ ID NO: 1.

In some embodiments, the BBB transporters comprise a Vu comprising residues Y28, R36, Y37, D57, S59, V62, E64, R108, L109, P110, Y111, Y112 and Y113, optionally further comprising S26 and/or Y38; and a V_{L} comprising residues F38, Y56, and R66; wherein the residue positions are according to IMGT numbering. In certain embodiments, said BBB transporters bind to an epitope of hTfR comprising residues K261, T491, S492, N493, F494, K495, H515, V517, T518, Q520, L522, Q524, D525, N527, S530, K531, E533, D560, and E582 of SEQ ID NO: 1, and optionally further comprising K358 and/or D525 of SEQ ID NO: 1. In particular embodiments, when the BBB transporters are bound to hTfR,
S26 is within 4.0 Å of residue K261 of SEQ ID NO: 1;
S26 is within 4.0 Å of residue K358 of SEQ ID NO: 1;
Y28 is within 4.0 Å of residue K261 of SEQ ID NO: 1;
R36 is within 4.0 Å of residue K495 of SEQ ID NO: 1;
R36 is within 4.0 Å of residue K531 of SEQ ID NO: 1;
R36 is within 4.0 Å of residue E533 of SEQ ID NO: 1;
Y37 is within 4.0 Å of residue N493 of SEQ ID NO: 1;
Y37 is within 4.0 Å of residue D560 of SEQ ID NO: 1;
Y38 is within 4.0 Å of residue D525 of SEQ ID NO: 1
D57 is within 4.0 Å of residue N527 of SEQ ID NO: 1;
D57 is within 4.0 Å of residue K531 of SEQ ID NO: 1;
S59 is within 4.0 Å of residue S530 of SEQ ID NO: 1;
S59 is within 4.0 Å of residue K531 of SEQ ID NO: 1;
V62 is within 4.0 Å of residue N527 of SEQ ID NO: 1;
V62 is within 4.0 Å of residue S530 of SEQ ID NO: 1;
E64 is within 4.0 Å of residue N527 of SEQ ID NO: 1;
R108 is within 4.0 Å of residue N493 of SEQ ID NO: 1;
R108 is within 4.0 Å of residue D560 of SEQ ID NO: 1;
L109 is within 4.0 Å of residue S492 of SEQ ID NO: 1;
L109 is within 4.0 Å of residue D560 of SEQ ID NO: 1;
P110 is within 4.0 Å of residue S492 of SEQ ID NO: 1;
P110 is within 4.0 Å of residue N493 of SEQ ID NO: 1;
Y111 is within 4.0 Å of residue S492 of SEQ ID NO: 1;
Y111 is within 4.0 Å of residue F494 of SEQ ID NO: 1;
Y111 is within 4.0 Å of residue H515 of SEQ ID NO: 1;
Y111 is within 4.0 Å of residue T518 of SEQ ID NO: 1;
Y111 is within 4.0 Å of residue L522 of SEQ ID NO: 1;
Y112 is within 4.0 Å of residue T491 of SEQ ID NO: 1;
Y112 is within 4.0 Å of residue S492 of SEQ ID NO: 1;
Y112 is within 4.0 Å of residue H515 of SEQ ID NO: 1;
Y112 is within 4.0 Å of residue V517 of SEQ ID NO: 1;
Y112 is within 4.0 Å of residue T518 of SEQ ID NO: 1;
Y113 is within 4.0 Å of residue Q520 of SEQ ID NO: 1;
Y113 is within 4.0 Å of residue Q524 of SEQ ID NO: 1;
F38 is within 4.0 Å of residue Q520 of SEQ ID NO: 1;
Y56 is within 4.0 Å of residue V517 of SEQ ID NO: 1;
Y56 is within 4.0 Å of residue T518 of SEQ ID NO: 1;
R66 is within 4.0 Å of residue E582 of SEQ ID NO: 1;
or any combination thereof (e.g., any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or all 38 thereof). In certain embodiments, UCSF Chimera, PISA, or UCSF ChimeraX software, or any combination thereof, is used for the visualization of the paratope/epitope structure and the determination of the distance in Å and interactions between paratope residues and epitope residues. Additionally or alternatively to the 4.0 Å distance embodiments above, in particular embodiments, when the BBB transporters are bound to hTfR,
S26 can form a hydrogen bond with residue K261 of SEQ ID NO: 1;
R36 can form a hydrogen bond with residue K531 of SEQ ID NO: 1;
Y37 can form a hydrogen bond with residue N493 of SEQ ID NO: 1;
Y38 can form a hydrogen bond with residue D525 of SEQ ID NO: 1;
D57 can form a hydrogen bond with residue N527 of SEQ ID NO: 1;
D57 can form a salt bridge with residue K531 of SEQ ID NO: 1;
S59 can form a hydrogen bond with residue K531 of SEQ ID NO: 1;
R108 can form a hydrogen bond with residue N493 of SEQ ID NO: 1;
R108 can form a salt bridge with residue D560 of SEQ ID NO: 1;
Y111 can form a hydrogen bond with residue S492 of SEQ ID NO: 1;
Y111 can form a hydrogen bond with residue L522 of SEQ ID NO: 1;
Y113 can form a hydrogen bond with residue Q524 of SEQ ID NO: 1;
Y56 can form a hydrogen bond with residue V517 of SEQ ID NO: 1;
Y56 can form a hydrogen bond with residue T518 of SEQ ID NO: 1;
R66 can form a salt bridge with residue E582 of SEQ ID NO: 1;
or any combination thereof (e.g., any 1, 2, 3, 4, 5, 6, 7, 8, 9, 10, 11, 12, 13, or all 14 thereof).

In some embodiments, a BBB transporter herein comprises a paratope comprising VI amino acid residues (i.e., residues in the V_{H}) R36, Y37, D57, S59, R108, Y111 and Y113; and V_{L} amino acid residues (i.e., residues in the VL) Y56 and R66; wherein the numbering is according to IMGT. In some embodiments, the paratope further comprises V_{H} amino acid residue S26. In some embodiments, the paratope further comprises V_{H} amino acid residue Y38 (or W38). In some embodiments, the paratope further comprises V_{H} amino acid residues S26 and Y38 (or W38). In some embodiments, the paratope further comprises at least one V_{H} amino acid residue selected from: Y28, V62, E64, L109, P110 and Y112. In some embodiments, the paratope further comprises V_{H} amino acid residue Y28. In some embodiments, the paratope further comprises V_{H} amino acid residue V62. In some embodiments, the paratope further comprises V_{H} amino acid residue E64. In some embodiments, the paratope further comprises V_{H} amino acid residue L109. In some embodiments, the paratope further comprises V_{H} amino acid residue P110. In some embodiments, the paratope further comprises V_{H} amino acid residue Y112. In some embodiments, the paratope further comprises V_{H} amino acid residues S26, Y28, Y38 (or W38), V62, E64, L109, P110 and Y112. In some embodiments, the paratope further comprises V_{H} amino acid residues Y28, V62, E64, L109, P110 and Y112. In some embodiments, the paratope further comprises V_{L} amino acid residue F38. In some embodiments, the paratope further comprises V_{H} amino acid residues S26, Y28, Y38 (or W38), V62, E64, L109, P110 and Y112 and V_{L} amino acid residue F38. In some embodiments, the paratope further comprises V_{H} amino acid residues Y28, V62, E64, L109, P110 and Y112 and V_{L} amino acid residue F38.

In some embodiments, the BBB transporter comprises a paratope comprising in the V_{H} at least nine (e.g., at least 10, at least 11, at least 12, at least 13, or at least 14) amino acid residues selected from the group consisting of S26, Y28, R36, Y37, Y38 (or W38), D57, S59, V62, E64, R108, L109, P110, Y111, Y112 and Y113; and/or in the VI, at least two amino acid residues selected from the group consisting of F38, Y56 and R66; wherein the numbering is according to IMGT.

In some embodiments, the BBB transporter comprises a paratope comprising at least one (e.g., 1, 2, 3, 4, 5, 6, 7, 8, 9, 10, 11, 12, 13, 14 or 15) amino acid residue selected from the group consisting of V_{H} amino acid residues S26, Y28, R36, Y37, Y38 (or W38), D57, S59, V62, E64, R108, L109, P110, Y111, Y112 and Y113, wherein the numbering is according to IMGT. In some embodiments, the BBB transporter comprises a paratope comprising at least one (e.g., 1, 2 or 3) amino acid residue selected from the group consisting of V_{L} amino acid residues F38, Y56 and R66, wherein the numbering is according to IMGT.

In some embodiments, the BBB transporter comprises a paratope comprising (i) at least one (e.g., 1, 2, 3, 4, 5, 6, 7, 8, 9, 10, 11, 12, 13, 14 or 15) amino acid residue selected from the group consisting of V_{H} amino acid residues S26, Y28, R36, Y37, Y38 (or W38), D57, S59, V62, E64, R108, L109, P110, Y111, Y112 and Y113; and (ii) at least one (e.g., 1, 2 or 3) amino acid residue selected from the group consisting of V_{L} amino acid residues F38, Y56 and R66, wherein the numbering is according to IMGT. In some embodiments the numbering is according to a different system (Kabat, Chothia, Aho, AbM, or combinations thereof) and the positions are adapted accordingly.

In some embodiments, the BBB transporter comprises a paratope comprising the V_{H} amino acid residues Y28, R36, Y37, D57, S59, V62, E64, R108, L109, P110, Y111, Y112 and Y113; and optionally further comprising S26 and/or Y38 (or W38), wherein the numbering is according to IMGT. In some embodiments, the BBB transporter comprises a paratope comprising the V_{L} amino acid residues F38 and Y56 and optionally further comprising R66, wherein the numbering is according to IMGT. In some embodiments, the BBB transporter comprises a paratope comprising the V_{H} amino acid residues Y28, R36, Y37, D57, S59, V62, E64, R108, L109, P110, Y111, Y112 and Y113; and optionally further comprising S26 and/or Y38 (or W38); and (ii) the V_{L} amino acid residues F38 and Y56 and optionally further comprising R66, wherein the numbering is according to IMGT. In some embodiments, the numbering is according to a different system (Kabat, Chothia, Aho, AbM, or combinations thereof) and the positions are adapted accordingly.

In some embodiments, the BBB transporter comprises an HCDR1 comprising Y28, R36, Y37 and Y38 (or W38); an HCDR2 comprising D57, S59, V62 and E64; an HCDR3 comprising R108, L109, P110, Y111, Y112, Y113; an LCDR1 comprising F38; an LCDR2 comprising Y56; and an LFR3 starting with an arginine residue (R66). In some embodiments, the BBB transporter comprises an HCDR1 comprising Y28, R36, Y37 and Y38 (or W38); an HCDR2 comprising D57, S59, V62 and E64; and an HCDR3 comprising R108, L109, P110, Y111, Y112, Y113. In some embodiments, the BBB transporter comprises an LCDR1 comprising F38; an LCDR2 comprising Y56; and an LFR3 starting with an arginine residue (R66).

In some embodiments, the BBB transporter comprises
a V_{H} comprising
optionally an HFR1 ending with a serine residue (S26);
an HCDR1 comprising Y28, R36, Y37 and optionally Y38 (or W38), and comprising 1-5 additional amino acid residues of SEQ ID NO: 26;
an HCDR2 comprising D57, S59, V62 and E64, and comprising 1-4 additional amino acid residues of SEQ ID NO: 28; and
an HCDR3 comprising R108, L109, P110, Y111, Y112, Y113, and comprising 1-7 additional amino acid residues of SEQ ID NO: 7; and/or a V_{L} comprising
an LCDR1 comprising F38 and comprising 1-5 additional amino acid residues of SEQ ID NO: 29;
an LCDR2 comprising Y56 and comprising 1-2 additional amino acid residues of SEQ ID NO: 10;
an LFR3 starting with an arginine residue (R66); and
optionally an LCDR3 comprising 1-9 amino acid residues of SEQ ID NO: 11.

In some embodiments, the BBB transporter comprises a V_{H} comprising
optionally an HFR1 ending with a serine residue (S26);
an HCDR1 comprising Y28, R36, Y37 and optionally Y38 (or W38), and comprising 1-5 additional amino acid residues of SEQ ID NO: 26;
an HCDR2 comprising D57, S59, V62 and E64, and comprising 1-4 additional amino acid residues of SEQ ID NO: 28; and
an HCDR3 comprising R108, L109, P110, Y111, Y112, Y113, and comprising 1-7 additional amino acid residues of SEQ ID NO: 7.

In some embodiments, the BBB transporter comprises a V_{L} comprising an LCDR1 comprising F38 and comprising 1-5 additional amino acid residues of SEQ ID NO: 29;
an LCDR2 comprising Y56 and comprising 1-2 additional amino acid residues of SEQ ID NO: 10;
an LFR3 starting with an arginine residue (R66); and
optionally a LCDR3 comprising 1-9 amino acid residues of SEQ ID NO: 11.

In some embodiments, the BBB transporter comprises
a V_{H} comprising
optionally an HFR1 ending with a serine residue (S26);
an HCDR1 comprising R36 and Y37, additionally comprising 1-6 amino acid residues of SEQ ID NO: 26;
an HCDR2 comprising D57 and S59, additionally comprising 1-6 amino acid residues of SEQ ID NO: 28;
an HCDR3 comprising R108, Y111, Y113, additionally comprising 1-10 amino acid residues of SEQ ID NO: 7; and/or
a V_{L} comprising
an LCDR2 comprising Y56, additionally comprising 1-2 amino acid residues of SEQ ID NO: 10;
an LFR3 starting with an arginine residue (R66); and
optionally an LCDR3 comprising 1-9 amino acid residues of SEQ ID NO: 11.

In certain embodiments, the number of amino acid residues in the CDRs of the BBB transporter is the same as indicated in the respective SEQ ID NOs., i.e., HCDR1: 8 residues, HCDR2: 8 residues, HCDR3: 13 residues, LCDR1: 6 residues, LCDR2: 3 residues and LCDR3 (when present): 9 residues.

In some embodiments, the BBB transporter comprises further to a set of paratope residues specified herein HCDR1, HCDR2 and HCDR3 having sequences according to SEQ ID NOs: 26, 28 and 7, respectively. In some embodiments, the BBB transporter comprises further to a set of paratope residues specified herein at least one (e.g., 1, 2 or 3) HCDRs selected from an HCDR1, HCDR2 and HCDR3 having sequences according to SEQ ID NOs: 26, 28 and 7, respectively. In some embodiments, the BBB transporter comprises further to a set of paratope residues specified herein an LCDR1 and LCDR2 having sequences according to SEQ ID NOs: 29 and 10, respectively; optionally further comprising an LCDR3 having a sequence according to SEQ ID NO: 11. In some embodiments, the BBB transporter comprises further to a set of paratope residues specified herein at least one (e.g., 1, 2 or 3) LCDRs selected from LCDR 1, LCDR2 and LCDR3 having sequences according to SEQ ID NOs: 29, 10 and 11, respectively.

In some embodiments, a BBB transporter is provided that comprises further to a set of paratope residues specified herein an HCDR1, HCDR2 and HCDR3 having sequences according to SEQ ID NOs: 26, 28 and 7, respectively, optionally further comprising an LCDR1 and LCDR2 having sequences according to SEQ ID NOs: 29 and 10, respectively; and optionally an LCDR3 having a sequence according to the sequence of SEQ ID NO: 11. In some embodiments, the BBB transporter comprises further to a set of paratope residues specified herein at least one (e.g., 1, 2 or 3) HCDRs selected from HCDR1, HCDR2 and HCDR3 having sequences according to SEQ ID NOs: 26, 28 and 7, respectively, optionally further comprising at least one (e.g., 1, 2 or 3) LCDRs selected from LCDR1, LCDR2 and LCDR3 having sequences according to SEQ ID NOs: 29, 10 and 11, respectively. The CDR sequences disclosed in the above embodiments have been annotated according to IMGT. In some embodiments the numbering is according to a different system (Kabat, Chothia, Aho, AbM, or combinations thereof) and the sequence of the CDRs are adapted accordingly. In some embodiments, the BBB transporter further comprises an HFR1 ending with a serine residue (i.e., a serine residue at the carboxy terminal end of the HFR1). In some embodiments, the BBB transporter comprises further to a set of paratope residues specified herein an LFR3 starting with an arginine residue (i.e., an arginine residue at the amino terminal start of the LFR3). In some embodiments, the BBB transporter comprises further to a set of paratope residues specified herein an HFR1 ending with a serine residue and an LFR3 starting with an arginine residue. In some embodiments, the HFR1 has a sequence at least 50% identical to the HFR1 sequence comprised in SEQ ID NO: 21, optionally with the proviso that the HFR1 ends with a serine residue. In some embodiments, the LFR3 has a sequence at least 50% identical to the LFR3 sequence comprised in SEQ ID NO: 25, optionally with the proviso that the LFR3 starts with an arginine residue. In some embodiments, the framework regions have the sequences as comprised in SEQ ID NOs: 21 and 25. The framework sequences disclosed in the above embodiments have been annotated according to IMGT. In some embodiments, the numbering is according to a different system (Kabat, Chothia, Aho, AbM, or combinations thereof) and the sequences of the framework regions are adapted accordingly.

In some embodiments, the BBB transporter comprises further to a set of paratope residues specified herein a Vu having a sequence at least 50% (at least 60%, at least 70%, at least 80%, at least 85%, at least 90%, at least 95%, at least 97%, at least 99%) identical to the sequence of SEQ ID NO: 21; and a V_{L} having a sequence at least 50% (at least 60%, at least 70%, at least 80%, at least 85%, at least 90%, at least 95%, at least 97%, at least 99%) identical to the sequence of SEQ ID NO: 25.

In some embodiments, the BBB transporter comprises further to a set of paratope residues specified herein a V_{H} having a sequence at least 95% identical to the sequence of SEQ ID NO: 21; and a V_{L} having a sequence at least 95% identical to the sequence of SEQ ID NO: 25. In some embodiments, the BBB transporter comprises further to a set of paratope residues specified herein a V_{H} having a sequence at least 50% (at least 60%, at least 70%, at least 80%, at least 85%, at least 90%, at least 95%, at least 97%, at least 99%) identical to the sequence of SEQ ID NO: 21. In some embodiments, the BBB transporter comprises further to a set of paratope residues specified herein a V_{H} having a sequence at least 95% identical to the sequence of SEQ ID NO: 21. In some embodiments, the BBB transporter comprises further to a set of paratope residues specified herein a V_{L} having a sequence at least 50% (at least 60%, at least 70%, at least 80%, at least 85%, at least 90%, at least 95%, at least 97%, at least 99%) identical to the sequence of SEQ ID NO: 25. In some embodiments, the BBB transporter comprises further to a set of paratope residues specified herein a V_{L} having a sequence at least 95% identical to the sequence of SEQ ID NO: 25.

In some embodiments, the BBB transporter comprises further to a set of paratope residues specified herein a V_{H} having a sequence according to SEQ ID NO: 21; and a V_{L} having a sequence according to SEQ ID NO: 25. In some embodiments, the BBB transporter comprises further to a set of paratope residues specified herein a Vu having a sequence according to SEQ ID NO: 21. In some embodiments, the BBB transporter comprises further to a set of paratope residues specified herein a V_{L} having a sequence according to SEQ ID NO: 25.

In some embodiments, the above specified paratope residues of the BBB transporter may form hydrogen bonds and/or salt bridges with epitope residues when bound to hTfR (according to SEQ ID NO: 1). In some embodiments the following paratope residues, when present, can form a hydrogen bond with at least one epitope residue of hTfR when bound, wherein the paratope residues within the V_{H} are:
a) S26 that can form a hydrogen bond with residue K261 of SEQ ID NO: 1;
b) R36 that can form a hydrogen bond with residue K531 of SEQ ID NO: 1;
c) Y37 that can form a hydrogen bond with residue N493 of SEQ ID NO: 1;
d) Y38 that can form a hydrogen bond with residue D525 of SEQ ID NO: 1.
e) D57 that can form a hydrogen bond with residue N527 of SEQ ID NO: 1;
f) S59 that can form a hydrogen bond with residue K531 of SEQ ID NO: 1;
g) R108 that can form a hydrogen bond with residue N493 of SEQ ID NO: 1;
h) Y111 that can form a hydrogen bond with residue S492 and residue L522 of SEQ ID NO: 1; and
i) Y113 that can form a hydrogen bond with residue Q524 and Q520 of SEQ ID NO: 1; and
wherein the paratope residue within the V_{L} is:
j) Y56 that can form a hydrogen bond with residue V517 and a hydrogen bond with residue T518 of SEQ ID NO: 1.

In some embodiments, the following paratope residues, when present, can form a salt bridge with at least one epitope residue of hTfR when bound, wherein the paratope residues within the Vu are
a) R108 that can form a salt bridge with residue N560 of SEQ ID NO: 1; and
b) D57 that can form a salt bridge with residue K531 of SEQ ID NO: 1; and wherein the paratope residues within the V_{L} is:
j) R66 that can form a salt bridge with residue E582 of SEQ ID NO: 1.

In some embodiments, the following paratope residues, when present, can form a hydrogen bond and/or a salt bridge with at least one epitope residue of hTfR when bound, wherein the paratope residues within the V_{H} are:
a) S26 that can form a hydrogen bond with residue K261 of SEQ ID NO: 1;
b) R36 that can form a hydrogen bond with residue K531 of SEQ ID NO: 1;
c) Y37 that can form a hydrogen bond with residue N493 of SEQ ID NO: 1;
d) Y38 that can form a hydrogen bond with residue D525 of SEQ ID NO: 1.
e) D57 that can form a hydrogen bond with residue N527 of SEQ ID NO: 1;
f) D57 that can form a salt bridge with residue K531 of SEQ ID NO: 1;
g) S59 that can form a hydrogen bond with residue K531 of SEQ ID NO: 1;
h) R108 that can form a hydrogen bond with residue N493 of SEQ ID NO: 1;
i) R108 that can form a salt bridge with residue N560 of SEQ ID NO: 1;
j) Y111 that can form a hydrogen bond with residue S492 and residue L522 of SEQ ID NO: 1; and
k) Y113 that can form a hydrogen bond with residue Q524 and Q520 of SEQ ID NO: 1; and
wherein the paratope residue within the VL is:
l) Y56 that can form a hydrogen bond with residue V517 and a hydrogen bond with residue T518 of SEQ ID NO: 1; and
m) R66 that can form a hydrogen bond with residue E582 of SEQ ID NO: 1.

In some embodiments, the above specified paratope residues of the BBB transporter may have additional properties when bound to hTfR (according to SEQ ID NO: 1):
the following paratope residues located within the V_{H}, when present, may have the following additional properties when bound to hTfR (according to SEQ ID NO: 1):
   a) S26 is within 4.0 Å of residue K261 and K358 of SEQ ID NO: 1;
   b) Y28 is within 4.0 Å of residue K261 of SEQ ID NO: 1;
   c) R36 is within 4.0 Å of residues K495, K531, and E533 of SEQ ID NO: 1;
   d) Y37 is within 4.0 Å of residues N493 and D560 of SEQ ID NO: 1;
   e) Y38 is within 4.0 Å of residue D525 of SEQ ID NO: 1
   f) D57 is within 4.0 Å of residues N527 and K531 of SEQ ID NO: 1;
   g) S59 is within 4.0 Å of residues S530 and K531 of SEQ ID NO: 1;
   h) V62 is within 4.0 Å of residues N527 and S530 of SEQ ID NO: 1;
   i) E64 is within 4.0 Å of residue N527 of SEQ ID NO: 1;
   j) R108 is within 4.0 Å of residues N493 and D560 of SEQ ID NO: 1;
   k) L109 is within 4.0 Å of residues S492 and D560 of SEQ ID NO: 1;
   l) P110 is within 4.0 Å of residues S492 and N493 of SEQ ID NO: 1;
   m) Y111 is within 4.0 Å of residues S492, F494, H515, T518, and L522 of SEQ ID NO: 1;
   n) Y112 is within 4.0 Å of residues T491, S492, H515, V517, and T518 of SEQ ID NO: 1; and
   o) Y113 is within 4.0 Å of residues Q520 and Q524 of SEQ ID NO: 1;
and
the following paratope residues located within the V_{L}, when present, may have the following additional properties when bound to hTfR (according to SEQ ID NO: 1):
   p) F38 is within 4.0 Å of residue Q520 of SEQ ID NO: 1;
   q) Y56 is within 4.0 Å of residues V517 and T518 of SEQ ID NO: 1; and
   r) R66 is within 4.0 Å of residue E582 of SEQ ID NO: 1.

The paratope residues disclosed in the above embodiments have been annotated according to IMGT. In some embodiments the numbering is according to a different system (Kabat, Chothia, Aho, AbM, or combinations thereof) and the position of the paratope residues are adapted accordingly.

The present disclosure provides, for example, the following numbered embodiments for an anti-TfR antibody or antigen-binding fragment thereof of the invention:
1 An anti-human transferrin receptor (hTfR) antibody or an antigen-binding fragment thereof, wherein the antibody or the antigen-binding fragment thereof comprises a paratope comprising heavy chain variable region (V_{H}) amino acid residues R36, Y37, D57, S59, R108, Y111 and Y113; and
light chain variable region (V_{L}) amino acid residues Y56 and R66;
wherein the numbering is according to IMGT.
2. The antibody or antigen-binding fragment of embodiment 1, wherein the paratope further comprises:
   V_{H} amino acid residues S26 and/or Y38 (or W38);
wherein the numbering is according to IMGT.
3. The antibody or antigen-binding fragment according to any one of the previous embodiments, wherein the paratope further comprises:
   V_{H} amino acid residues Y28, V62, E64, L109, P110 and Y112; and
   V_{L} amino acid residue F38;
wherein the numbering is according to IMGT.
4. The antibody or antigen-binding fragment according to any one of the previous embodiments, comprising:
a V_{H} comprising
   a heavy chain CDR (HCDR) 1 comprising Y28, R36, Y37 and optionally Y38 (or W38), and comprising 1-5 additional amino acid residues of SEQ ID NO: 26;
   an HCDR2 comprising D57, S59, V62 and E64, and comprising 1-4 additional amino acid residues of SEQ ID NO: 28; and
   an HCDR3 comprising R108, L109, P110, Y111, Y112, and Y113, and comprising 1-7 additional amino acid residues of SEQ ID NO:7; and/or
a V_{L} comprising
   a light chain CDR (LCDR) 1 comprising F38, and comprising 1-5 additional amino acid residues of SEQ ID NO: 29;
   an LCDR2 comprising Y56, and comprising 1-2 additional amino acid residues of SEQ ID NO: 10;
   a light chain framework region (LFR) 3 starting with an arginine residue (R66); and
   optionally an LCDR3 comprising 1-9 amino acid residues of SEQ ID NO: 11;
optionally wherein said antibody or antigen-binding fragment further comprises a heavy chain framework region (HFR) 1 ending with a serine residue, wherein the numbering is according to IMGT.
5. An anti-human transferrin receptor (hTfR) antibody or an antigen-binding fragment thereof, wherein the antibody or antigen-binding fragment comprises:
   heavy chain CDR (HCDR) 1, HCDR2 and HCDR3 having sequences according to SEQ ID NOs: 26, 28 and 7, respectively;
   light chain CDR (LCDR) 1 and LCDR2 having sequences according to SEQ ID NOs: 29 and 10, respectively; and optionally LCDR3 having a sequence according to SEQ ID NO: 11;
   a heavy chain framework region (HFR) 1 ending with a serine residue; and
   a light chain framework region (LFR) 3 starting with an arginine residue.
6. The antibody or antigen-binding fragment according to any one of the previous embodiments, comprising:
   a V_{H} having a sequence at least 50% identical to the sequence of SEQ ID NO: 21; and
   a V_{L} having a sequence at least 50% identical to the sequence of SEQ ID NO: 25.
7. The antibody or antigen-binding fragment according to any one of the previous embodiments, wherein the following paratope residues, when present, can form a hydrogen bond with at least one epitope residue of hTfR when bound, wherein the paratope residues within the V_{H} are:
   a) S26 that can form a hydrogen bond with residue K261 of SEQ ID NO: 1;
   b) R36 that can form a hydrogen bond with residue K531 of SEQ ID NO: 1;
   c) Y37 that can form a hydrogen bond with residue N493 of SEQ ID NO: 1;
   d) Y38 that can form a hydrogen bond with residue D525 of SEQ ID NO: 1.
   e) D57 that can form a hydrogen bond with residue N527 of SEQ ID NO: 1;
   f) S59 that can form a hydrogen bond with residue K531 of SEQ ID NO: 1;
   g) R108 that can form a hydrogen bond with residue N493 of SEQ ID NO: 1;
   h) Y111 that can form a hydrogen bond with residue S492 and residue L522 of SEQ ID NO: 1; and
   i) Y113 that can form a hydrogen bond with residue Q524 and Q520 of SEQ ID NO: 1; and
wherein the paratope residue within the V_{L} is:
   j) Y56 that can form a hydrogen bond with residue V517 and a hydrogen bond with residue T518 of SEQ ID NO: 1, wherein the numbering is according to IMGT.
8. The antibody or antigen-binding fragment according to any one of the previous embodiments, wherein the following paratope residues, when present, can form a salt bridge with at least one epitope residue of hTfR when bound, wherein the paratope residues within the V_{H} are
   a) R108 that can form a salt bridge with residue D560 of SEQ ID NO: 1; and
   b) D57 that can form a salt bridge with residue K531 of SEQ ID NO: 1; and wherein the paratope residues within the V_{L} is:
   c) R66 that can form a hydrogen bond with residue E582 of SEQ ID NO: 1, wherein the numbering is according to IMGT.
9. The antibody or antigen-binding fragment according to any one of the previous embodiments, wherein the following paratope residues, when present, are within 4.0 Å of at least one epitope residue of hTfR when bound, wherein the paratope residues within the V_{H} are:
   a) S26 that is within 4.0 Å of residue K261 and K358 of SEQ ID NO: 1;
   b) Y28 that is within 4.0 Å of residue K261 of SEQ ID NO: 1;
   c) R36 that is within 4.0 Å of residues K495, K531, and E533 of SEQ ID NO: 1;
   d) Y37 that is within 4.0 Å of residues N493 and D560 of SEQ ID NO: 1;
   e) Y38 that is within 4.0 Å of residue D525 of SEQ ID NO: 1
   f) D57 that is within 4.0 Å of residues N527 and K531 of SEQ ID NO: 1;
   g) S59 that is within 4.0 Å of residues S530 and K531 of SEQ ID NO: 1;
   h) V62 that is within 4.0 Å of residues N527 and S530 of SEQ ID NO: 1;
   i) E64 that is within 4.0 Å of residue N527 of SEQ ID NO: 1;
   j) R108 that is within 4.0 Å of residues N493 and D560 of SEQ ID NO: 1;
   k) L109 that is within 4.0 Å of residues S492 and D560 of SEQ ID NO: 1;
   l) P110 that is within 4.0 Å of residues S492 and N493 of SEQ ID NO: 1;
   m) Y111 that is within 4.0 Å of residues S492, F494, H515, T518, and L522 of SEQ ID NO: 1;
   n) Y112 that is within 4.0 Å of residues T491, S492, H515, V517, and T518 of SEQ ID NO: 1; and
   o) Y113 that is within 4.0 Å of residues Q520 and Q524 of SEQ ID NO: 1; and wherein the paratope residues within the V_{L} are:
   p) F38 that is within 4.0 Å of residue Q520 of SEQ ID NO: 1;
   q) Y56 that is within 4.0 Å of residues V517 and T518 of SEQ ID NO: 1; and
   r) R66 that is within 4.0 Å of residue E582 of SEQ ID NO: 1,
wherein the numbering is according to IMGT.

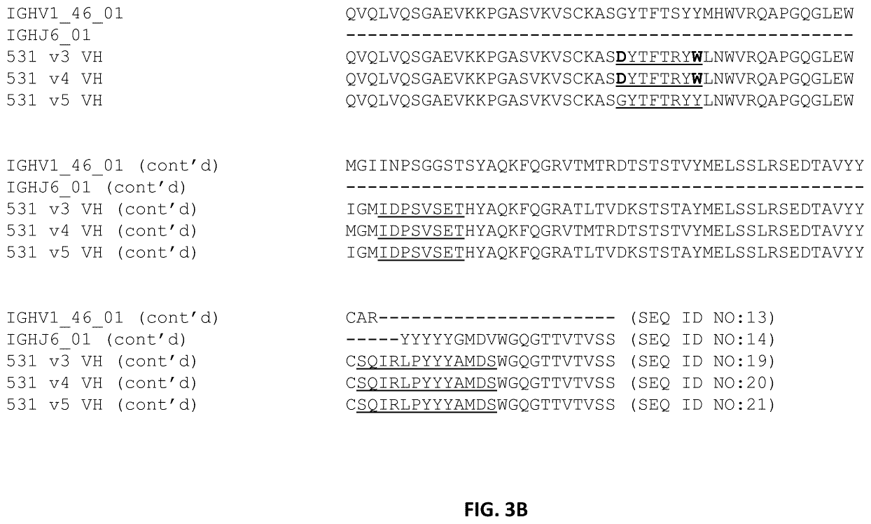

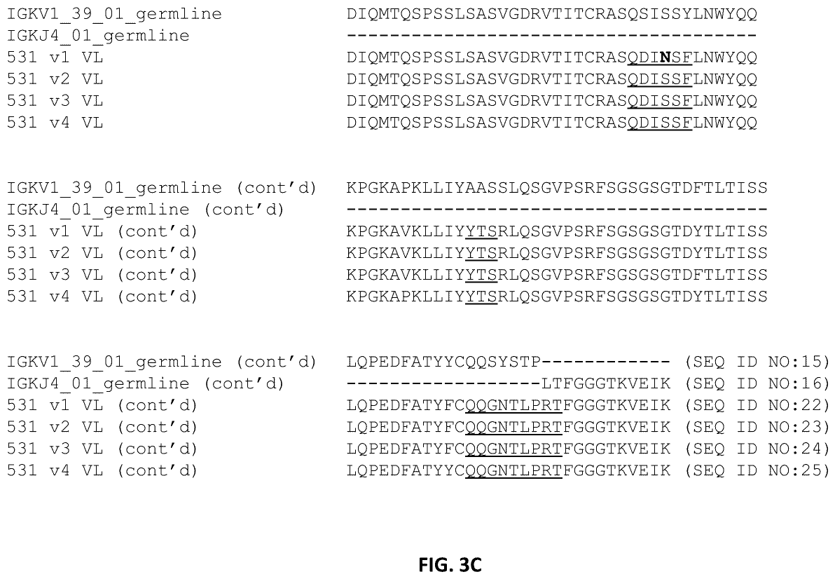

In some embodiments, the BBB transporter comprises the HCDRs described herein inserted into human heavy chain framework sequences derived from human germline sequences IGVH3-23*05 or IGHV1-46*01 and IGHJ6_01. Exemplary humanized V_{H} sequences generated with these human germline sequences are shown in FIG. 3A and FIG. 3B.

In some embodiments, the BBB transporter comprises the LCDRs described herein inserted in human kappa light chain framework sequences derived from human germline sequences IGKV1-39*01 and IGKJ4-01. Exemplary humanized V_{L} sequences generated with these human germline sequences are shown in FIG. 3C.

In some embodiments, the BBB transporter comprises a V_{H} comprising any one of SEQ ID NOs: 17-21, or an amino acid sequence that is at least 90% (e.g., at least 91, 92, 93, 94, 95, 96, 97, 98, or 99%) identical thereto; and/or a V_{L} comprising any one of SEQ ID NOs: 22-25, or an amino acid sequence that is at least 90% (e.g., at least 91, 92, 93, 94, 95, 96, 97, 98, or 99%) identical thereto. In further embodiments, the BBB transporter comprises a V_{H} and a V_{L} comprising
SEQ ID NOs: 21 and 25,
SEQ ID NOs: 20 and 25,
SEQ ID NOs: 19 and 25,
SEQ ID NOs: 18 and 25,
SEQ ID NOs: 17 and 25,
SEQ ID NOs: 21 and 24,
SEQ ID NOs: 20 and 24,
SEQ ID NOs: 19 and 24,
SEQ ID NOs: 18 and 24,
SEQ ID NOs: 17 and 24,
SEQ ID NOs: 21 and 23,
SEQ ID NOs: 20 and 23,
SEQ ID NOs: 19 and 23,
SEQ ID NOs: 18 and 23,
SEQ ID NOs: 17 and 23,
SEQ ID NOs: 21 and 22,
SEQ ID NOs: 20 and 22,
SEQ ID NOs: 19 and 22,
SEQ ID NOs: 18 and 22,
SEQ ID NOs: 17 and 22, or
SEQ ID NOs: 4 and 8,
respectively, or amino acid sequences that are at least 90% (e.g., at least 91, 92, 93, 94, 95, 96, 97, 98, or 99%) identical to the above-listed SEQ ID NO pairs. In some embodiments, the variant V_{H} and V_{L} sequences comprise the HCDRs and LCDRs (defined by, e.g., IMGT) in the above-listed SEQ ID NO pairs and the sequence variations occur in the framework regions. In some embodiments, the variant V_{H} and V_{L} retain the properties of their parental sequences in terms of TfR-binding properties.

In further embodiments, the BBB transporter comprises a V_{H} and a V_{L} comprising
SEQ ID NOs: 21 and 25,
SEQ ID NOs: 21 and 24,
SEQ ID NOs: 21 and 23,
SEQ ID NOs: 19 and 23,
SEQ ID NOs: 18 and 23,
SEQ ID NOs: 17 and 23,
SEQ ID NOs: 21 and 22,
SEQ ID NOs: 20 and 22,
SEQ ID NOs: 18 and 22,
SEQ ID NOs: 17 and 22, or
SEQ ID NOs: 4 and 8,
respectively, or amino acid sequences that are at least 90% (e.g., at least 91, 92, 93, 94, 95, 96, 97, 98, or 99%) identical to the above-listed SEQ ID NO pairs. In some embodiments, the variant V_{H} and V_{L} sequences comprise the HCDRs and LCDRs (defined by, e.g., IMGT) in the above-listed SEQ ID NO pairs and the sequence variations occur in the framework regions. In some embodiments, the variant V_{H} and V_{L} retain the properties of their parental sequences in terms of TfR-binding properties.

The percent identity of two amino acid sequences (or of two nucleic acid sequences) may be obtained by, e.g., BLAST® using default parameters (available at the U.S. National Library of Medicine's National Center for Biotechnology Information website). The length of a reference sequence aligned for comparison purposes is at least 30%, (e.g., at least 40, 50, 60, 70, 80, or 90% of the reference sequence. For V_{H}, V_{L}, HC, or LC sequence identity or homology, the percent identity and homology is calculated based on the alignment of the full-length query and reference V_{H}, V_{L}, HC, or LC sequences.

The present disclosure also contemplates BBB transporters structurally defined according to any of the above embodiments, with any combination of functional properties described herein for BBB transporters.

### 2. Formats of BBB Transporters

In some embodiments, the BBB transporters herein take on the format of full, tetrameric antibodies. The antibodies may be of any immunoglobulin isotype, such as IgG (e.g., IgG1, IgG2, IgG3, or IgG4). The antibodies herein preferably comprise a human IgG (e.g., IgG1) constant region. In some embodiments, the IgG constant region may comprise mutations that improve the clinical potential of the antibody, such as mutations that reduce or eliminate effector functions (ADCC and/or CDC) of the antibody (see, e.g., Wang et al., Protein Cell (2018) 9(1):63-73). For example, the monospecific or multi-specific antibody herein may comprise a human IgG₁ constant region with the mutation L235E, “LALA” mutations (L234A/L235A), or “LALAGA” mutations (L234A/L235A/G237A) (Eu numbering). In some embodiments, the heavy chains comprise one, two, or all three mutations of S298N, T299A, and Y300S (“NNAS” mutations).

The IgG constant region may comprise mutations that improve the serum half-life of the antibody, such as the M428L mutation, the M252Y/S254T/T256E (“YTE” mutations), and mutations described in WO 2019/147973.

To facilitate proper protein assembly of the BBB during manufacturing, the antibody heavy chains (e.g., IgG₁ heavy chains) may comprise knob-in-hole mutations (e.g., Y349C, T366S, L368A, and Y407V for IgG₁ hole mutations; and S354C and T366W for IgG₁ knob mutations). In some embodiments, the hole heavy chain comprises H435R/Y436F (“RF”) double mutations, which enable easy removal of hole-hole homodimers and hole half-IgG by-products during manufacturing.

All such mutated human constant regions are still considered “human” constant regions herein. Unless otherwise indicated, all residue numbers in IgG constant regions are Eu numbers.

In some embodiments, the anti-TfR antibody comprises an HC comprising SEQ ID NO: 32, or an amino acid sequence that is at least 90% (e.g., at least 91, 92, 93, 94, 95, 96, 97, 98, or 99%) identical thereto; and/or an LC comprising SEQ ID NO: 31, or an amino acid sequence that is at least 90% (e.g., at least 91, 92, 93, 94, 95, 96, 97, 98, or 99%) identical thereto. In some embodiments, the anti-TfR antibody comprises an HC comprising SEQ ID NO: 32 and an LC comprising SEQ ID NO: 31 (531v25 mAb). In some embodiments, the variant HC and LC sequences comprise the HCDRs and LCDRs (defined by, e.g., IMGT) in SEQ ID NOs: 32 and 31 and the sequence variations occur in the framework and/or constant regions. In some embodiments, the variant HC and LC retain the properties of their parental sequences in terms of TfR-binding properties.

The BBB transporters may also be antigen-binding fragments of full antibodies. In some embodiments, the transporters are monovalent for TfR (i.e., each transporter has only one binding site for TfR) and/or comprise a Fab. For example, the BBB transporter may be a Fab comprising a HC comprised of a V_{H} and a CHI (e.g., an IgG CHI) and an LC comprised of a V_{L} and a C_{L} (e.g., a kappa C_{L}). This transporter is also termed “Fab transporter” herein. In some embodiments, the Fab transporter comprises an HC comprising SEQ ID NO: 30, or an amino acid sequence that is at least 90% (e.g., at least 91, 92, 93, 94, 95, 96, 97, 98, or 99%) identical thereto; and/or an LC comprising SEQ ID NO: 31, or an amino acid sequence that is at least 90% (e.g., at least 91, 92, 93, 94, 95, 96, 97, 98, or 99%) identical thereto. In some embodiments, the Fab transporter comprises an HC comprising SEQ ID NO: 30 and an LC comprising SEQ ID NO: 31 (531v25Fab). In some embodiments, the Fab transporter comprises an HC comprising a V_{H} as set forth in SEQ ID NO: 21 and an LC comprising a V_{L} as set forth in SEQ ID NO: 25. In some embodiments, the variant HC and LC sequences comprise the HCDRs and LCDRs (defined by, e.g., IMGT) in SEQ ID NOs: 30 and 31 (or SEQ ID NOs: 21 and 25) and the sequence variations occur in the framework and/or constant regions. In some embodiments, the variant HC and LC retain the properties of their parental sequences in terms of TfR-binding properties.

In some embodiments, the BBB transporter resembles a full IgG antibody except that it has only one TfR-binding domain. Such a BBB transporter is a heterotrimer comprising an LC, a full antibody HC, and an Fc polypeptide that dimerizes with the constant region of the full antibody HC to form a Fc domain. This transporter is also termed “Fab-Fc” or “Fab-FcOL” transporter herein. In an IgG, the Fc domain is a disulfide-bonded dimeric structure formed by the CH₂ and CH₃ of an HC pair. An “Fc polypeptide” refers herein to a polypeptide that can dimerize through disulfide bonds to form an Fc domain and yet does not have the V_{H} portion of a full antibody HC. In some embodiments, the Fc polypeptide contains the CH₂ and CH₃ and at least a part of the hinge region of a human IgG (e.g., human IgG₁, IgG₂, or IgG₄), such that it is capable of dimerizing with a full human IgG (e.g., human IgG₁, IgG₂, or IgG₄) heavy chain to form a human IgG₁, IgG₂, or IgG₄ Fc domain.

In some embodiments, the Fc polypeptide and the full heavy chain may comprise one or more of the mutations discussed above that improve the clinical potential of the BBB transporter, such as mutations that reduce or eliminate effector functions (e.g., “LALA” and “NNAS” mutations), mutations that improve the serum half-life of the BBB transporter (e.g., “YTE” mutations and mutations described in WO 2019/147973), mutations that facilitate proper protein assembly of the BBB transporter (e.g., knob-in-hole mutations), and mutations that improve purification of the BBB transporter during manufacturing (e.g., “RF” mutations).

Exemplary TfR-binding antibody fragments include the following molecules:
(i) Fab-FcOL TfR-binding antibody fragment comprising an HC comprising a V_{H} as set forth in SEQ ID NO: 21, an LC comprising a V_{L} as set forth in SEQ ID NO: 25, and an Fc polypeptide (optionally as set forth in SEQ ID NO: 34);
(ii) Fab-FcOL TfR-binding antibody fragment comprising an HC comprising SEQ ID NO: 30, an LC comprising SEQ ID NO: 31, and an Fc polypeptide (optionally as set forth in SEQ ID NO: 34);
(iii) Fab-FcOL TfR-binding antibody fragment comprising an HC comprising SEQ ID NO: 33, an LC comprising SEQ ID NO: 31, and an Fc polypeptide comprising SEQ ID NO: 34 (531v25Fab-FcOL); and
(iv) Fab-FcOL TfR-binding antibody fragment comprising an HC comprising SEQ ID NO: 56, an LC comprising SEQ ID NO: 31, and an Fc polypeptide comprising SEQ ID NO: 34 (53 1v25Fab-FcOL with engineered cysteine).

Further exemplary BBB transporters include the following molecules: (i) an antibody or an antigen-binding fragment thereof comprising an HC comprising HCDR1-3 as set forth in SEQ ID NOs: 26, 28, and 7, respectively, and an LC comprising LCDR1-3 as set forth in SEQ ID NOs: 29, 10, and 11, respectively (and optionally an Fc polypeptide); (ii) an antibody or an antigen-binding fragment thereof comprising a V_{H} as set forth in SEQ ID NO: 21 and a V_{L} as set forth in SEQ ID NO: 25 (and optionally an Fc polypeptide); and (iii) an antibody or an antigen-binding fragment thereof comprising an HC comprising SEQ ID NO: 30 and an LC comprising SEQ ID NO: 31 (and optionally an Fc polypeptide). These BBB transporters are herein referred to as “531v25 BBB transporters.” 531v25 BBB transporters having the Fab-FcOL format as defined herein are referred as “531v25 Fab-FcOL BBB transporters.”

### C. Cargos

The present BBB transporters (including the 531v25 BBB transporters) can be used to transport a variety of cargos across the BBB to the brain for diagnostic, prophylactic, and therapeutic purposes. The cargo may be chemically conjugated to a transporter, e.g., through a lysine or cysteine residue in the transporter. In some embodiments, the cargo may be chemically conjugated to an engineered residue, e.g., an engineered cysteine residue (THIOMAB™ technology) or an engineered lysine residue.

Alternatively, the cargo, if it is a peptide or polypeptide, may be recombinantly fused to the transporter. For example, when the transporter is a full antibody transporter (having two light chains and full-length heavy chains), such as the 531v25mAb, the cargo may be fused to the N- or C-terminus of one or both of the light chains, or to the N- or C-terminus of one or both of the heavy chains. In further embodiments, the cargo may be fused to the C-terminus of one of the heavy chains (e.g., the knob heavy chain, or the hole heavy chain).

In some embodiments, the transporter is a Fab transporter (such as the 531v25Fab) and the cargo may be fused to the N- or C-terminus of the light chain, or to the N- or C-terminus of the heavy chain. In further embodiments, the cargo may be fused to the C-terminus of the heavy chain. In certain embodiments, the cargo may be fused to the C-terminus of the light chain.

In some embodiments, the transporter is a Fab-FcOL transporter (such as the 531v25Fab-FcOL) and the cargo may be fused to the N- or C-terminus of the light chain, to the N- or C-terminus of the full heavy chain, or to the N- or C-terminus of the Fc polypeptide. In further embodiments, the cargo may be fused to the C-terminus of the Fc polypeptide. In certain embodiments, the cargo may be fused to the C-terminus of the light chain.

The cargo may be linked to the anti-TfR antibody or antigen-binding fragment thereof through a peptide linker. In some embodiments, the peptide linker may predominantly include the following amino acid residues: Gly, Ser, Ala, or Thr. The peptide linker may have a length that is adequate to link two molecules in such a way that they assume the correct conformation relative to one another so that they retain their respective desired activity. In some embodiments, the linker is 1 to 50 (e.g., 1 to 30, 1 to 20, 1 to 10 or 1 to 5) amino acids in length. Useful linkers include glycine-serine polymers, including for example, (GS)n, (GSGGS)n (SEQ ID NO: 44), (GGGGS)n (SEQ ID NO: 45), and (GGGS)n (SEQ ID NO: 46), where n is an integer of at least one; glycine-alanine polymers; alanine-serine polymers; XTEN linkers; and other flexible linkers. In some embodiments, the linker is GGGG (SEQ ID NO: 42) or SGSGGGG (SEQ ID NO: 43). Additional exemplary linkers for linking antibody fragments or single-chain variable fragments can include AAEPKSS (SEQ ID NO: 47), AAEPKSSDKTHTCPPCP (SEQ ID NO: 48), GGGG (SEQ ID NO: 42), or GGGGDKTHTCPPCP (SEQ ID NO: 49).

The cargos transportable by the present BBB transporters (including 531v25 BBB transporters) may be diagnostic agents, e.g., imaging agents for the brain.

The cargos transportable by the present BBB transporters (including the 531v25 BBB transporters) may be therapeutic agents. Exemplary therapeutic agents are peptides/polypeptides and oligonucleotides.

In some embodiments, the therapeutic agent is an enzyme. In certain embodiments, the enzyme is a lysosomal enzyme. In further embodiments, the enzyme is acid alpha-glucosidase (GAA), e.g., a recombinant human GAA. GAA is also known as a-1,4-glucosidase and acid maltase. It is an enzyme that helps to break down glycogen in the lysosome. An example of recombinant human GAA is alglucosidase alfa (Myozyme® and Lumizyme®). In some embodiments, the recombinant GAA comprises or consists of SEQ ID NO: 35.

In some embodiments, the present TfR-binding protein for transporting GAA is a Fab-FcOL transporter comprising an HC, an LC and an Fc polypeptide, and a human GAA sequence fused to the C-terminus of (a) the HC, (b) the LC, or (c) the Fc polypeptide, wherein the heavy chain and the Fc polypeptide dimerize to form an Fc domain. In further embodiments, the present TfR-binding protein for transporting GAA comprises an HC, an LC, an Fc polypeptide, and a human GAA sequence fused to the C-terminus of the LC, wherein the heavy chain and the Fc polypeptide dimerize to form an Fc domain. In further embodiments, the TfR-binding protein for transporting GAA is a 531v25 Fab-FcOL BBB transporter comprising an HC, an LC and an Fc polypeptide, wherein the human GAA sequence is fused to the C-terminus of the LC. In further embodiments, the TfR-binding protein is a Fab-FcOL comprising three polypeptides comprising SEQ ID NOs: 33, 36, and 34, respectively (531v25Fab-FcOL-LC-GAA). In other embodiments, the present TfR-binding protein for transporting GAA comprises an HC, an LC, an Fc polypeptide, and a human GAA sequence fused to the C-terminus of the Fc polypeptide, wherein the heavy chain and the Fc polypeptide dimerize to form an Fc domain. In further embodiments, the TfR-binding protein for transporting GAA is a 531v25 Fab-FcOL BBB transporter comprising an HC, an LC and an Fc polypeptide, wherein the human GAA sequence is fused to the C-terminus of the Fc polypeptide. In further embodiments, the TfR-binding protein is a Fab-FcOL comprising three polypeptides comprising SEQ ID NOs: 33, 31, and 37, respectively (531v25Fab-FcOL-GAA).

In some embodiments, the present TfR-binding protein for transporting GAA comprises a Fab and a human GAA sequence fused to the C-terminus of the HC or LC of the Fab. In further embodiments, the present TfR-binding protein for transporting GAA comprises a Fab and a human GAA sequence fused to the C-terminus of the LC of the Fab. In further embodiments, the TfR-binding protein for transporting GAA is a 531v25 BBB transporter comprising an HC and an LC in a Fab format, wherein the human GAA sequence is fused to the C-terminus of the LC. In further embodiments, the TfR-binding protein comprises two polypeptides comprising SEQ ID NOs: 30 and 36, respectively (531v25Fab-GAA).

In some embodiments, the present TfR-binding protein for transporting GAA comprises two HCs, two LCs, and a human GAA sequence fused to the C-terminus of one of the two HCs. In further embodiments, the present TfR-binding protein for transporting GAA is a 531v25 BBB transporter comprising two HCs, two LCs, and a human GAA sequence fused to the C-terminus of one of the two HCs. In further embodiments, the two LCs each comprise SEQ ID NO: 31, one of the HCs comprises SEQ ID NO: 33, and the other HC comprises SEQ ID NO: 37 (531v25mAb-GAA).

In some embodiments, the therapeutic agent is an oligonucleotide. In certain embodiments, the oligonucleotide is an antisense oligonucleotide or an siRNA, e.g., targeting a CNS gene.

In some embodiments, the present TfR-binding protein for transporting an oligonucleotide is a Fab-FcOL transporter comprising an HC, an LC and an Fc polypeptide, wherein the heavy chain and the Fc polypeptide dimerize to form an Fc domain, and wherein an oligonucleotide is conjugated to a residue (e.g., a lysine or cysteine) in one of said polypeptides. In certain embodiments, the oligonucleotide is conjugated to an engineered residue (e.g., an engineered cysteine residue introduced through THIOMAB™ technology, or an engineered lysine residue) for site-specific conjugation. In further embodiments, the TfR-binding protein for transporting the oligonucleotide is a 531v25 Fab-FcOL BBB transporter comprising an HC, an LC and an Fc polypeptide, e.g., wherein the oligonucleotide is conjugated to one of said transporter polypeptides as described above. In further embodiments, the TfR-binding protein is a Fab-FcOL comprising three polypeptides comprising SEQ ID NOs: 31, 34, and 56, respectively.

In some embodiments, the present TfR-binding protein for transporting an oligonucleotide comprises a Fab. In further embodiments, the TfR-binding protein for transporting an oligonucleotide is a 531v25 BBB transporter comprising an HC and an LC in a Fab format, wherein an oligonucleotide is conjugated to a residue (e.g., a lysine or cysteine) of the HC or LC. In certain embodiments, the oligonucleotide is conjugated to an engineered residue (e.g., an engineered cysteine residue introduced through THIOMAB™ technology, or an engineered lysine residue) for site-specific conjugation.

In some embodiments, the present TfR-binding protein for transporting an oligonucleotide comprises two HCs, two LCs, and an oligonucleotide conjugated to a residue (e.g., a lysine or cysteine) of one of said HCs and LCs. In certain embodiments, the oligonucleotide is conjugated to an engineered residue (e.g., an engineered cysteine residue introduced through THIOMAB™ technology, or an engineered lysine residue) for site-specific conjugation. In further embodiments, the present TfR-binding protein for transporting an oligonucleotide is a 531v25 BBB transporter comprising two HCs, two LCs, and an oligonucleotide conjugated to one of said HCs and LCs as described above. In certain embodiments, the two LCs each comprise SEQ ID NO: 31, one of the HCs comprises SEQ ID NO: 33, and the other HC comprises SEQ ID NO: 56. In certain embodiments, the two LCs each comprise SEQ ID NO: 31, and the two HCs each comprise SEQ ID NO: 56.

It is to be understood that the linkage of a cargo to the BBB transporters herein does not affect the TfR-binding properties of the transporters. A cargo-loaded BBB transporter has the binding properties (e.g., TfR-binding affinity) as described in Section A, supra.

### II. Making of TfR-Binding Proteins

The TfR-binding proteins described herein may be produced recombinantly using isolated nucleic acid molecules such as expression constructs encoding each chain of the proteins. Biomolecules (e.g., nucleic acid or polypeptide molecules) referred to herein as “isolated” or “purified” are those that (1) have been separated away from the biomolecules (e.g., nucleic acids of the genomic DNA or cellular RNA, or polypeptides, of their source of origin; and/or (2) do not occur in nature. The encoding sequences for each polypeptide chain may be cloned into a single vector or cloned into separate vectors.

Methods of producing proteins such as antibodies are well known. The present binding proteins such as antibodies may be produced in, e.g., mammalian host cells, using appropriate expression constructs. Mammalian cell lines available as hosts for expression include many immortalized cell lines available from the American Type Culture Collection (ATCC). These include, inter alia, Chinese hamster ovary (CHO) cells, NS0 cells, SP2 cells, HEK-293T cells, 293 Freestyle cells (Invitrogen), NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Other cell lines that may be used are insect cell lines, such as Sf9 or Sf21 cells, and yeast cell lines. Cell lines may be selected based on their expression levels. The binding proteins may be isolated and purified from the host cell culture using well known methods, such as centrifugation, ultracentrifugation, protein A, protein G, protein A/G, or protein L purification, and/or ion exchange chromatography.

### III. Pharmaceutical Compositions and Use

The present disclosure also provides pharmaceutical compositions comprising the TfR-binding proteins herein. The pharmaceutical compositions may comprise one or more pharmaceutically acceptable excipients, carriers, or diluents. As used herein, “pharmaceutically acceptable” with reference to a “carrier,” “excipient,” or “diluent” includes appropriate solvents, dispersion media, antibacterial and antifungal agents, isotonic agents, and the like. In some embodiments, the pharmaceutical composition is a sterile aqueous solution, and may comprise a buffer; a surfactant; a polyol; an antioxidant; and/or a chelating agent.

The pharmaceutical composition comprising a BBB transporter herein loaded with an enzyme (e.g., a lysosomal enzyme) is useful in treating a human patient with, or at risk of developing, an enzyme deficiency (e.g., a lysosomal enzyme deficiency). In some embodiments, the enzyme or an activity of the enzyme is deficient (e.g., insufficient in quantity or even entirely missing) in the patient. In some embodiments, the GAA transporters herein may be used to treat patients who need higher GAA activity levels, e.g., patients who are deficient in GAA congenitally. In some embodiments, the GAA BBB transporters may be used to restore GAA activity. For example, the GAA BBB transporters are used to treat Pompe disease in human patients in need thereof. Pompe disease (aka. acid a-glucosidase deficiency, acid maltase deficiency, glycogen storage disease type II, GSD II, and glycogenosis type II) is an inherited disorder of glycogen metabolism caused by the absence or marked deficiency of the lysosomal enzyme GAA. The GAA transporters herein can be used to treat late onset Pompe disease (LOPD) and/or infantile onset Pompe Disease (IOPD).

The pharmaceutical composition comprising a BBB transporter herein loaded with an oligonucleotide (e.g., an ASO or siRNA) is useful in treating a human patient who will benefit from transport of the oligonucleotide to, e.g., the CNS. In some embodiments, the BBB transporter can be used to transport the oligonucleotide to a specific, non-CNS target tissue that is rich in TfR expression, such as in the peripheral nervous system (e.g., sciatic nerve), skeletal muscle, an internal organ (e.g., heart or spleen), or another tissue. Where the oligonucleotide is an ASO or siRNA, the oligonucleotide-loaded BBB transporter may be useful in treating a human patient who will benefit from knockdown of a target of the ASO or siRNA.

As used herein, the terms “treat,” “treatment,” and “treating” refers to a deliberate intervention to a physiological disease state resulting in the reduction in severity of a disease or condition; the reduction in the duration of a disease or condition; the amelioration or elimination of one or more symptoms associated with a disease or condition; or the provision of beneficial effects to a subject with a disease or condition. Treatment does not require curing the underlying disease or condition.

The pharmaceutical composition may be provided to the patient at a dosage strength and a frequency determined as appropriate by a health care provider. Therapeutically effective amounts are those sufficient to ameliorate one or more symptoms associated with the disease or affliction to be treated. A “therapeutically effective amount,” “effective dose,” “effective amount,” or “therapeutically effective dosage” of the binding protein herein protects a subject against the onset of a disease or promotes disease regression or stabilization as evidenced by a decrease in severity of disease symptoms, an increase in frequency and duration of disease symptom-free periods, or a prevention or delay of impairment or disability due to the disease affliction.

The present disclosure also provides the use of the present antibodies or antigen-binding fragments for the manufacture of a medicament comprising a therapeutic molecule, wherein the medicament is capable of crossing the BBB. In some embodiments, the present disclosure provides the use of the present enzyme-loaded TfR-binding proteins for the manufacture of a medicament for treating a subject deficient in the enzyme (e.g., GAA-loaded TfR-binding proteins for treating Pompe disease). In some embodiments, the present disclosure provides the use of oligonucleotide-loaded TfR-binding proteins for the manufacture of a medicament for treating a subject in need thereof, e.g., wherein the oligonucleotide may be an ASO or siRNA.

The present disclosure also provides the use of the present antibodies or antigen-binding fragments for diagnostic processes (e.g., in vitro or ex vivo). For example, the antibodies and antigen-binding fragments can be used to detect and/or measure the level of TfR in a biological sample from a patient (e.g., a tumor biopsy, a tissue sample, or a blood sample). Suitable detection and measurement methods include immunological methods such as flow cytometry, enzyme-linked immunosorbent assays (ELISA), chemiluminescence assays, radioimmunoassays, and immunohistochemistry. The present disclosure further encompasses kits (e.g., diagnostic kits) comprising the antibodies or antigen-binding fragments described herein.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

### Example 1: Generation and Characterization of Mouse 531 mAb

A. Protocol on m531design: 531-11.2_mouse

The purpose of this study was to generate and identify an antibody that binds TfR. To do so, a mouse was immunized with human tissue expressing human TfR. A hybridoma was then selected and the sequences of its antibody, designated 531-1L2_mouse, were obtained by RT-PCR and sequencing. The V_{H} and V_{L} sequences of the 531-1L2_mouse mAb are set forth in SEQ ID NOs: 4 and 8, respectively. The HCDRs are set forth in SEQ ID NOs: 5-7 and the LCDRs are set forth in SEQ ID NOs: 9-11.

B. Affinity measurement of 531-11.2_mouse by SPR

Binding of 531-1L2_mouse Ab to human, cynomolgus, and mouse TfR was assessed using SPR at pH7.4. Experiments were run on Bruker MASS-2 instrument with HBS-EP+ as running buffer and sample diluent (Cytiva Life Sciences, #BR100826). HCA sensorchips (Bruker, #1862614) were immobilized with anti his antibody (His capture kit, Cytiva Life Sciences, #28995056) following provider's instructions. About 10 000 RU of anti his was obtained. His-tagged human transferrin receptor proteins—human TfR ectodomain of SEQ ID NO: 39, cynomolgus TfR ectodomain of SEQ ID NO: 40 or mouse TfR ectodomain of SEQ ID NO: 41 (5 μg/ml)—were captured for 1 min at 10 μL/min. Concentration series of the 531-1L2_mouse Ab were then injected for 4 min at 30 μL/min over human transferrin receptor surfaces and dissociation was monitored for 5 min. Surface was regenerated with one 1 min pulse of 10 mM Glycine-HCl pH1.5. Bruker Sierra Analyzer software was used for analysis: Sensorgrams were double referenced with reference surface (bulk and weak non-specific binding subtraction) and blank (drift removal) and curves were fitted with 1:1 binding model

This experiment confirms that the 531-1L2_mouse antibody binds to the TfR ectodomain. The binding affinities for TfR are shown in Table 1 below.

TABLE 1
Assessment of affinity of 531_1L2-mouse for TfR by SPR

| | | | mTfR | hTfR | cTfR | |
|---|---|---|---|---|---|---|
| | | | K_{D} | K_{D} | K_{D} | Cynomolgus/ |
| Format | Analyte | Technique | (nM) | (nM) | (nM) | human |
| Bivalent | 531_1L2- | SPR | 0 | 6.9 | 27 | 3.9 |
| | mouse | | | | | |

C. Competition of 531-1L2_mouse antibody with transferrin by SPR

The purpose of this study was to determine whether the 531-1L2_mouse antibody impacts the binding of the transferrin to its receptor.

Experiments were conducted on BIAcore™ T200 instrument with HBS-EP+ as running buffer and sample diluent (Cytiva Life Sciences, #BR100826). About 500 RU of 531-1L2_mouse antibody was covalently immobilized on C1 sensorchip (Cytiva Life Sciences, #BR100540) using amine coupling kit (Cytiva Life Sciences, #BR100050). 3 min injection of 50 nM of hTfR (R&D Systems, #2474-TR; SEQ ID NO: 1 with the G142S SNP substitution associated with the Caucasian population) over 531-1L2_mouse surface was immediately followed by a 3 min-injection of 100 nM of human transferrin (Sigma, #T4132; expressed as SEQ ID NO: 54 and for which the peptide signal is cleaved) at 10 μL/min. About 150 RU of hTfR bound to 531-1L2_mouse antibody and about 100 RU of human transferrin bound to hTfR were measured. 3 min injection of 50 nM of hTfR over 531-1L2_mouse surface was immediately followed by a 3 min injection of 100 nM of 531-1L2_mouse antibody at 10 μL/min. About 150 RU of hTfFR bound to 531-1L2 mouse was measured and no binding of 531-1L2_mouse could be detected (competition control). Surface was regenerated with 30s pulse of 10 mM glycine-HCl (pH 2.5) at 10 μL/min.

We observed that there was no competition between the 531-1L2_mouse antibody and transferrin for binding to its receptor.

### Example 2: Generation of Humanized Fab Antibodies

The purpose of this study was to generate humanized 531 variants (in Fab format) with optimal binding properties to hTfR and cTfR (i.e., with about 1 log or less between affinity for cTfR and affinity for hTfR).

A. Generation and expression of humanized Fab variants

The technique applied to design humanized Fab variants of the 531-1L2_mouse antibody was CDR grafting in which the CDRs of non-human antibodies are grafted onto human frameworks.

First, from the location of the CDRs identified within the V_{H} and V_{L} sequences of the 531-1L2_mouse antibody (SEQ ID NOs: 4 and 8), residues critical for the conformation of CDR loops, antibody structure, and affinity and specificity for human and cynomolgus TfR (e.g., Vernier residues, Contact residues and Anchors) were identified. Several human heavy chain germline (IGHV3-23*05 as set forth in SEQ ID NO: 12 and IGHV1-46*01 as set forth in SEQ ID NO: 13) and human light chain germline (IGKV1-39*01 as set forth in SEQ ID NO: 15) sequences, which were considered to provide frameworks closest to the mouse frameworks and to support the mouse CDR structure, were chosen as acceptors for CDR grafting.

Then the sequences of the human IMGT CDRs were replaced with the residues of the mouse IMGT CDRs. One or more of the above-identified critical residues were back-mutated (reversion to mouse sequence) and intra-CDR residue(s) were modified where appropriate to avoid chemical instability. Five humanized Vu sequences (SEQ ID NOs: 17-21) and four humanized V_{L} sequences (SEQ ID NOs: 22-25) were generated and combined to produce humanized Fab variants.

The humanized anti-TfR variants, in a Fab-His tag format, were expressed by transient transfection of HEK293 cells. The Fab proteins were purified in a two-steps process (IMAC & SEC) to achieve at least 95% of purity. Before affinity determination by SPR, the His-tag was removed by cleavage with the TEV protease.

### B. Affinity Measurement of Humanized Fab Variants to Various Species TfR by SPR

Binding of the humanized Fab variants to human, cynomolgus and mouse TfR was assessed using SPR at pH 7.4. Experiments were run on a Bruker MASS-2 instrument with HBS-EP+ as running buffer and sample diluent (Cytiva Life Sciences, #BR100826). HCA sensor chips (Bruker, #1862614) were immobilized with anti-His antibody (His capture kit, Cytiva Life Sciences, #28995056) following manufacturer's instructions. About 10,000 RU of anti-His was obtained. His-tagged hTfR ectodomain (SEQ ID NO: 39), cTfR ectodomain (SEQ ID NO: 40) or mouse TfR ectodomain (SEQ ID NO: 41) (5 μg/ml) was captured for 1 min at 10 μL/min. Concentration series of the humanized Fab variants were then injected for 4 min at 30 μL/min over hTfR surfaces and dissociation was monitored for 5 min. Surface was regenerated with one 1 min pulse of 10 mM glycine-HCl at pH 1.5. Bruker Sierra Analyzer software was used for analysis: Sensorgrams were double referenced with reference surface (bulk and weak non-specific binding subtraction) and blank (drift removal) and curves were fitted with 1:1 binding model.

Variant candidates were selected based on affinity for human and cynomolgus TfR, and cynomolgus/human ratio. This selection process yielded 10 variants (see Table 2; SEQ: SEQ ID NO; NB: no binding). None of the variants bound mouse TfR (mTfR). Next, we determined the V_{H} and V_{L} sequences of these 10 variants and their respective CDRs and combinations of CDRs. One variant (531v25) was selected for subsequent experiments (see Example 3).

TABLE 2
Binding Affinities of Mouse and Humanized Antibodies by SPR

| | | | | Affinity SPR K_{D} (nM) | | | |
|---|---|---|---|---|---|---|---|
| Antibody | Type | V_{H} SEQ | V_{L} SEQ | Mouse | Human | Cynomolgus | Cynomolgus/human |
| 531-1L2_mouse (mIgG1) | Mouse | 4 | 8 | NB | 6.93 | 27 | 3.9 |
| 531_1L2_variant3 Fab | Humanized | 17 | 22 | NB | 183 | 204 | 1.1 |
| 531_1L2_variant4 Fab | Humanized | 18 | 22 | NB | 120 | 1210 | 10.1 |
| 531_1L2_variant6 Fab | Humanized | 17 | 23 | NB | 77.4 | 300 | 3.9 |
| 531_1L2_variant8 Fab | Humanized | 18 | 23 | NB | 61.2 | 475 | 7.8 |
| 531_1L2_variant14 Fab | Humanized | 20 | 22 | NB | 8.6 | 75.1 | 8.7 |
| 531_1L2_variant16 Fab | Humanized | 21 | 22 | NB | 10.2 | 70.4 | 6.9 |
| 531_1L2_variant17 Fab | Humanized | 19 | 23 | NB | 5.91 | 28.9 | 4.9 |
| 531_1L2_variant21 Fab | Humanized | 21 | 23 | NB | 3.71 | 30.9 | 8.3 |
| 531_1L2_variant24 Fab | Humanized | 21 | 24 | NB | 10.2 | 84.6 | 8.3 |
| 531_1L2_variant25 Fab | Humanized | 21 | 25 | NB | 4.45 | 55.8 | 12.5 |

The HC and LC of the 531v25 Fab are set forth in SEQ ID NOs: 30 and 31, respectively. The V_{H} and V_{L} sequences of the 531v25 Fab are set forth in SEQ ID NOs: 21 and 25, respectively. The HCDR1-3 are set forth in SEQ ID NOs: 26, 28 and 7, respectively and the LCDR1-3 are set forth in SEQ ID NOs: 29, 10 and 11, respectively.

### Example 3: TfR-Binding Properties of 531v25-Derived Constructs

### A. Affinity Measurements

The purpose of this study was to compare the TfR-binding affinities of various 531v25-derived constructs: (1) 531v25Fab as in Example 2 and (2) 531v25Fab-FcOL (with LC as set forth in SEQ ID NO: 31, the HC as set forth in SEQ ID NO: 33 and the Fc polypeptide as set forth in SEQ ID NO: 34).

The affinities of the constructs to human and cynomolgus TfR (and ratio cynomolgus/human) were assessed using SPR and bio-layer interferometry (BLI) (see Table 3).

TABLE 3
Assessment of Affinity for TfR by SPR or BLI

| | | hTfR | cTfR | Cynomolgus/ |
|---|---|---|---|---|
| Analyte | Technique | K_{D} (nM) | K_{D} (nM) | human |
| 531v25-Fab | SPR | 4.5 | 55.8 | 12.4 |
| 531v25-Fab-FcOL | SPR | 11.8 | 124.3 | 10.5 |
| | SPR | 15 | 139.6 | 9.3 |
| | SPR | 8.5 | 109 | 12.8 |
| 531v25-Fab-FcOL | BLI | 22.1 | 103.4 | 4.7 |

Affinity by SPR was assessed as described in Example 2B, except that 531v25Fab or 531v25Fab-FcOL was used instead of the humanized Fab variants.

Affinity by BLI was assessed using an Octet® HTX instrument with the following conditions: the equilibrium dissociation constant (K_{D}) was determined on Octet® in HBSP buffer (10 mM HEPES, 150 mM NaCl, 0.05% polysorbate 20, pH 7.4). Streptavidin probes were used to immobilize a fixed concentration of the ligand: hTfR or cTfR, both with site-specific biotinylation on N-terminal Avitag; in this experiment, hTfR consists of an AviTag (SEQ ID NO: 55) fused to the N-terminus of the ectodomain of human TfR (residues 90 to 760 of SEQ ID NO: 1) and cTfR consists of the AviTag fused in the N-terminus of the ectodomain of cTfR (amino acid residues 90 to 760 of SEQ ID NO: 2). The ligand was tested for association and dissociation against the analyte (531v25-Fab-FcOL) in a 2-fold serial dilution series from 250 nM down to 3.91 nM. A 1:1 binding model with global fitting was applied after double subtraction for buffer and probe analyte interaction with a 0-90 second association and 0-60 dissociation window.

The SPR data confirm that the 531v25Fab-FcOL construct retained the affinity features of the 531v25Fab (including the cynomolgus/human affinity ratio). Additionally, BLI led to similar affinity determination for 531v25Fab-FcOL to human and cynomolgus monkey TfR, and therefore a similar affinity ratio as well.

B. No Competition with Transferrin of 531v25Fab-FcOL was Observed by BLI

We sought to confirm the absence of binding competition (to TfR) with transferrin, using BLI with saturation either by holoTF (holo-transferrin) or 531v25Fab-FcOL. The 531v25Fab-FcOL molecule was as detailed in Example 3A.

The equilibrium dissociation constant (K_{D}) was determined on Octet® in HBSP buffer (10 mM HEPES, 150 mM NaCl, 0.05% polysorbate 20, pH 7.4). Streptavidin probes were used to immobilize a fixed concentration of the ligand hTfR with site-specific biotinylation on N-terminal AviTag fused in the N-terminus of hTfR ectodomain. Subsequently, the immobilized hTfR was saturated with either 500 nM human holo-transferrin (#2914-HT, R&D systems) or 531v25Fab-FcOL. Subsequently, the potentially competing ligand was tested for association and dissociation against the blocked analyte (531v25Fab-FcOL) in a 2-fold serial dilution series from 500 nM down to 31.25 nM. A 1:1 binding model with global fitting was applied after double subtraction for buffer and probe analyte interaction with a 0-90 second association and 0-60 dissociation window. The ligand was tested for association and dissociation against the analyte (531v25Fab-FcOL) in a 2-fold serial dilution series from 250 nM down to 3.91 nM. A 1:1 binding model with global fitting was applied after double subtraction for buffer and probe analyte interaction.

The data show that transferrin binding was not affected by pre-association of 531v25Fab-FcOL. Holo-transferrin saturation to hTfR only minimally affected 531v25Fab-FcOL binding to hTfR. These data also indicate that there is no competition between 531v25Fab-FcOL and transferrin for binding to the transferrin receptor (see Table 4).

TABLE 4
Competition of 531v25-Fab-FcOL with
Transferrin for hTfR binding by BLI

| Analyte | KD (nM) |
|---|---|
| 531v25-Fab-FcOL | 22.1 |
| holoTF saturation then 531v25-Fab-FcOL | 44 |
| Holo-TF | 1.2 |
| 500 nM saturation 531v25-Fab-FcOL, then holoTF | 2.3 |

C. Biodistribution of Monovalent 531v25-Fab-FcOL after Single Dose in hTfR-KI Mice

hTfR-KI mice were dosed by intravenous injection with either 531v25-Fab-FcOL (as described above) or human IgG1 control Ab (Southern Biotech, Cat #0151K-14) at a dose of 70 nmol/kg. Mice were terminally anesthetized at 1, 3 and 24 h post dosing. Blood was collected in EDTA for plasma isolation, followed by perfusion (8 mL/min for 5 minutes) of the mice with Dulbecco's phosphate buffered saline solution containing heparin and Ca/Mg. Tissues were homogenized using Precellys® 2 mL hard tissue homogenizing ceramic bead kits in PBS containing 1% NP-40 Surfact Amps detergent (ThermoFisher) and Pierce protease inhibitor (A32955). Finally, anti-TfR construct concentration in the tissues (per wet tissue weight) was quantified by human IgG MSD (MSD human/NHP isotyping panel #K150JLD-1, Meso Scale Discovery) following the manufacturer's protocol.

Monovalent 531v25-Fab-FcOL had increased brain and spinal cord biodistribution (see Table 5), as well as faster plasma clearance (data not shown), in comparison to control IgG, confirming that the 531v25 binding domain is able to reach the brain and the CNS through the transferrin receptor.

TABLE 5
Brain and Spinal Cord Biodistribution
of 531v25-Fab-FcOL vs. IgG1

| | Ratio over IgG (brain) | Ratio over IgG (spinal cord) |
|---|---|---|
| 1 h | 5.3 | 4.5 |
| 3 h | 8.1 | 9.2 |
| 24 h | 22.4 | 29.4 |

### Example 4: Epitope and Paratope Mapping of 531v25 Fab

We sought to determine the localization of the human and cynomolgus TfR epitopes recognized by the 531v25 Fab using cryogenic electron microscopy (cryo-EM).

In this study, purified complexes of hTfR (SEQ ID NO: 39) or cTfR (SEQ ID NO: 40) with the 531v25 Fab were further purified using a Superdex200 3.3/300 column pre-equilibrated with PBS buffer. UltraAufoil® R 0.6/1 on 300 gold mesh grids were glow discharged at 22 mA for 45 s and 3 μL of each sample at the concentration of 1 and 0.64 mg/ml respectively were added to the grids and plunge frozen in liquid ethane. The data from the grids was collected on a Glacios™ microscope (Thermo Fisher) equipped with a Flacon4 camera at 200 keV. Images in the EER format were recorded with EPU at 240,000X nominal magnification at a pixel size of 0.58 Å and a range of defocus from −0.8 to −2.2. Dose on camera during an exposure time of 4.72 s was 60 e-/Å2, the number of fractions was 162 for 54 final frames.

A total of 7000 and 4630 images were taken in total for each grid respectively. The data analysis was carried out using CryoSPARC v3. The resolution of the final reconstructions was estimated at 2.5 Å and 2.73 Å for human and cynomolgus TfR respectively, using the value at which the FSC curve fell below 0.143. The two cryo-EM maps were sharpened using Phenix and then used to fit the atomic coordinates of the TfR and the 531v25 Fab. The atomic coordinates underwent several rounds of manual (on Coot) and real space refinement (in Phenix). Amino acid residues of hTfR and cTfR having atoms within 4 angstrom distance from 531v25 Fab atoms are represented on the diagrams of FIG. 1A and FIG. 1B, respectively. These distances were measured initially with UCSF Chimera software, and subsequently using PISA and UCSF ChimeraX software. The amino acid residues of hTfR and cTfR having atoms within 5 angstrom distance from 531v25 Fab atoms (as measured using UCSF Chimera software) are represented in the alignment below.

As shown in FIGS. 1A and 1B, the 531v25 Fab binds K261, K358, T491, S492, N493, K495, H515, V517, T518, Q520 (R520 for cTfR), Q524, D525, N527, S530, K531, E533, D560, and E582 of hTfR and cTfR within a 4 Å-resolution distance as determined by UCSF Chimera software. The 531v25 Fab binds K261, K358, T491, S492, N493, F494, K495, H515, V517, T518, Q520 (R520 for cTfR), L522, Q524, D525, N527, S530, K531, E533, D560, and E582 of hTfR and cTfR within a 4 Å-resolution distance as determined by PISA and UCSF ChimeraX software.

As shown in the alignment below, the 531v25 Fab binds K261, K287, K358, T491, S492, N493, F494, K495, M510, H515, V517, T518, Q520 (R520 for cTfR), F521 (S521 for cTfR), L522, Y523, Q524, D525, N527, S530, K531, V532, E533, E559, D560, D562 and E582 (boxed below) of hTfR and cTfR within a 5 Å-resolution distance.

| | |
|---|---|
| huTfR | GVEPKTECERLAGTESPVREEPGEDFPAARRLYWDDLKRKLSEKLDSTDFTGTIKLLNEN |
| cyTfR | GVEPKTECERLAGTESPAREEPEEDFPAAPRLYWDDLKRKLSEKLDTTDFTSTIKLLNEN |
| | ***********.******* ********* ****************:****.******** |
| huTfR | SYVPREAGSQKDENLALYVENQFREFKLSKVWRDQHFVKIQVKDSAQNSVIIVDKNGRLV |
| cyTfR | LYVPREAGSQKDENLALYIENQFREFKLSKVWRDQHFVKIQVKDSAQNSVIIVDKNGGLV |
| | *****************:************************************** ** |
| huTfR | 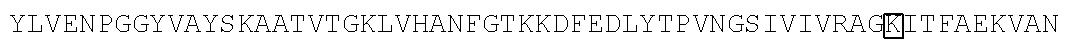 |
| cyTfR | 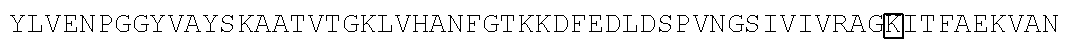 |
| | ************************************* :********************** |
| huTfR | 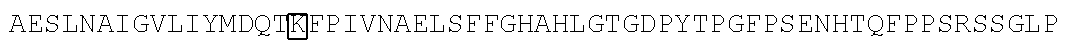 |
| cyTfR | 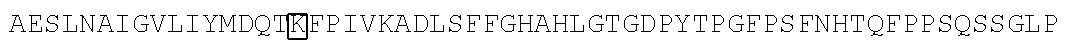 |
| | *********************:*:*******************************:***** |
| huTfR | 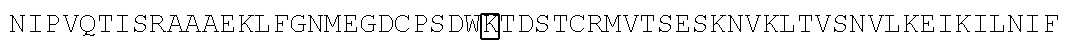 |
| cyTfR | 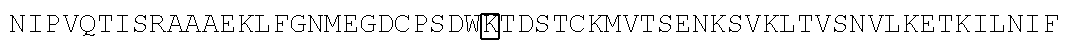 |
| | **********************************:*****.*.*********** ****** |
| huTfR | GVIKGFVEPDHYVVVGAQRDAWGPGAAKSGVGTALLLKLAQMFSDMVLKDGFQPSRSIIF |
| cyTfR | GVIKGFVEPDHYVVVGAQRDAWGPGAAKSSVGTALLLKLAQMFSDMVLKDGFQPSRSIIF |
| | *****************************.******************************* |
| huTfR | 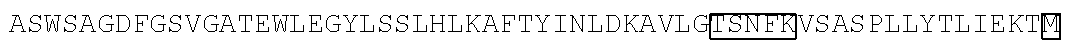 |
| cyTfR | 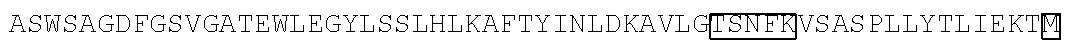 |
| | ************************************************************* |
| huTfR | 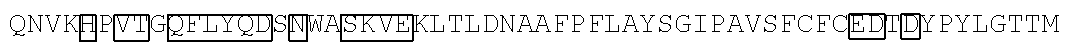 |
| CyTfR | 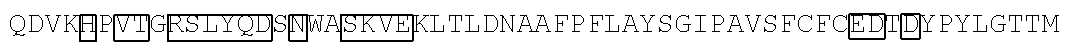 |
| | *:*******: ************************************************** |
| huTfR | 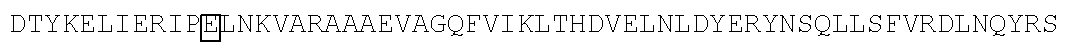 |
| cyTfR | 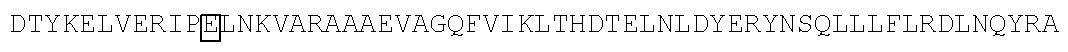 |
| | ******:***************************.*************** *:******** |
| huTfR | DIKEMGLSLOWLYSARGDFFRATSRLTTDFGNAEKTDRFVMKKLNDRVMRVEYHELSPYV |
| cyTfR | DVKEMGLSLOWLYSARGDFFRATSRLTTDFRNAEKRDKFVMKKLNDRVMRVEYYFLSPYV |
| | *:**************************** **** *:***************:****** |
| huTfR | SPKESPFRHVFWGSGSHTLPALLENLKLRKONNGAFNETLERNQLALATWTIQGAANALS |
| cyTfR | SPKESPFRHVFWGSGSHTLSALLESLKLRRONNSAFNETLERNQLALATWTIQGAANALS |
| | ******************* ****.****:***.************************** |
| huTfR | GDVWDIDNEF (residues 90-760 of SEQ ID NO: 1) |
| cyTfR | GDVWDIDNEF (residues 90-760 of SEQ ID NO: 2) |
| | ********** |

Interestingly, we observed that the 531v25 Fab binds to the protease-like domain (lateral) of the TfR, which corresponds to part of the TfR where there is less variability between human and cynomolgus. Two amino acid differences between human and cynomolgus TfR have been identified (at positions 520 and 521), which have been shown by Cryo-EM not to impact the interaction between the 531v25Fab and the TfR.

In parallel with the epitope residue determination, PISA and UCSF ChimeraX software were used to determine the 531v25 paratope residues as well as the interactions between these paratope residues and the epitope residues.

The following 13 residues of the V_{H} and 3 residues of the V_{L} were found to be within 4.0 Å of the epitope residues (human TfR): Y28, R36, Y37, D57, S59, V62, E64, R108, L109, P110, Y111, Y112 and Y113 in the V_{H} and F38, Y56, and R66 in the V_{L} (all residue positions are defined according to IMGT numbering). 12 of these residues are located in the CDRs: Y28, R36 and Y37 are located in the HCDR1; D57, S59, V62 and E64 are located in the HCDR2; R108, L109, P110, Y111, Y112 and Y113 are located in the HCDR3; F38 is located in the LCDR1; and Y56 is located in the LCDR2. One of the residues, R66, is located in LFR3.

Due to the flexibility of human TfR, two additional residues of the V_{H} (S26 located in the HFR1 and Y38 located in the HCDR1) were found to be within 4.0 Å of the epitope residues depending on the conformation of the hTfR monomers. S26 and Y38 can be within 4.0 Å of the epitope residues in one hTfR monomer conformation and slightly farther than 4.0 Å from the epitope residues in another hTfR monomer conformation. Thus, these two residues are considered to have weaker interactions with human TfR.

Interfacing residues within 4.0 Å were determined and are listed below:

### V_{H} of 531v25

Y28 (531v25 V_{H}) is within 4.0 Å of residue K261 of human TfR (SEQ ID NO: 1);
R36 is within 4.0 Å of residues K495, K531, and E533;
Y37 is within 4.0 Å of residues N493 and D560;
D57 is within 4.0 Å of residues N527 and K531;
S59 is within 4.0 Å of residues S530 and K531
V62 is within 4.0 Å of residues N527 and S530;
E64 is within 4.0 Å of residue N527;
R108 is within 4.0 Å of residues N493 and D560;
L109 is within 4.0 Å of residues S492 and D560;
P110 is within 4.0 Å of residues S492 and N493;
Y111 is within 4.0 Å of residues S492, F494, H515, T518, and L522;
Y112 is within 4.0 Å of residues T491, S492, H515, V517, and T518; and
Y113 is within 4.0 Å of residues Q520 and Q524.

### V_{L} of 531v25:

F38 (531v25 V_{L}) is within 4.0 Å of residue Q520 of human TfR (SEQ ID NO: 1);
Y56 is within 4.0 Å of residues V517 and T518; and
R66 is within 4.0 Å of residue E582.

Interfacing residues within 4.0 Å for the two additional residues of the V_{H} are as follows:
S26 (531v25 V_{H}) is within 4.0 Å of residues K261 and K358 of human TfR (SEQ ID NO: 1);
Y38 is within 4.0 Å of residue D525.

Among the interfacing residues within 4.0 Å as defined above, paratope residues involved in hydrogen bonds and salt bridges with epitope residues have been determined (hydrogen bonds and salt bridges being considered as strong interactions between the antibody and the antigen): R36, Y37, D57, S59, R108, Y111, and Y113 in the V_{H} and Y56 and R66 in the V_{L} (residue positions are defined according to IMGT numbering). In addition, residues S26 and Y38 in the V_{H} were also shown to form hydrogen bonds with epitope residues when within 4.0 Å. The following hydrogen bonds and salt bridges were determined:

### V_{H} of 531v25

R36 (531v25 V_{H}) can form a hydrogen bond with residue K531 of human TfR (SEQ ID NO: 1);
Y37 can form a hydrogen bond with residue N493;
D57 can form a hydrogen bond with residue N527 and a salt bridge with residue K531;
S59 can form a hydrogen bond with residue K531 of SEQ ID NO: 1;
R108 can form a hydrogen bond with residue N493 and a salt bridge with residue D560;
Y111 can form a hydrogen bond with residue S492 and a hydrogen bond with residue L522; and
Y113 can form a hydrogen bond with residue Q524.

### V_{L} of 531v25

Y56 (531v25 V_{L}) can form a hydrogen bond with residue V517 and a hydrogen bond with residue T518 of human TfR (SEQ ID NO: 1); and
R66 can form a salt bridge with residue E582.

Hydrogen bonds for the two additional residues in the V_{H}, when within 4.0 Å of the epitope residues, are as follows:
S26 (531v25 V_{H}) can form a hydrogen bond with residue K261 of human TfR (SEQ ID NO: 1); and
Y38 can form a hydrogen bond with residue D525.

### Example 5: Generation and Characterization of 531v25-GAA Molecules

Four anti-TfR-GAA molecules, having a fusion of GAA with different parts of anti-TfR binding entities, were constructed:
anti-TfR 531v25mAb-GAA: LC of SEQ ID NO: 31, HC of SEQ ID NO: 33 and HC-GAA fusion of SEQ ID NO: 38;
anti-TfR 531v25-Fab-LC-GAA: LC of SEQ ID NO: 36 and HC of SEQ ID NO: 30;
anti-TfR 531v25-Fab-FcOL-GAA: LC of SEQ ID NO: 31, HC of SEQ ID NO: 33 and Fc-GAA fusion of SEQ ID NO: 37; and
anti-TfR 531v25-Fab-FcOL-LC-GAA: LC of SEQ ID NO: 36, HC of SEQ ID NO: 33 and Fc polypeptide of SEQ ID NO: 34

### A. Affinity Measurements and GAA Activity

Next, we sought to compare the GAA activity and binding properties of these 4 anti-TfR-GAA molecules (see Table 6 for activity and ELISA data and Table 7 for SPR and BLI data). Recombinant GAA (SEQ ID NO: 35) was used as a control.

The specific (GAA) activity was measured with fluorometric enzymatic assay using synthetic substrate (4-Methylumbelliferyl a-D-glucopyranoside, M9766, Sigma). Briefly, standard curves (3.9-250 ng/ml) of GAA and anti-TfR-GAA constructs were prepared in dilution buffer (0.1% BSA, 0.2 M sodium acetate, 0.4 M KCl, pH 3.9). 15 μL of each standard sample (GAA or anti-TfR-GAA constructs) was mixed with 50 μL of 5 mM 4-Methylumbelliferyl a-D-glucopyranoside and incubated for 1 h at 37° C. The reaction was stopped with the addition of 135 μL of 1 M glycine-NaOH buffer (pH 12.5). Fluorescence of the reaction solution was measured (excitation at 360 nm and emission at 450 nm). A 4-Methylumbelliferone (M-1381, Sigma) standard curve (0.039-5 nmols/well) was fit by linear regression to calculate the amount of product. Specific activity (μmol product/min/mg GAA) was calculated by dividing the amount of product by the reaction time and amount of GAA.

Enzymatic activity of all tested molecules was in the range of 70-120% of GAA activity. This assay also showed similar activities of purified anti-TfR-GAA proteins compared to GAA (Table 6).

The binding kinetics of the four anti-TfR-GAA molecules to hTfR-expressing 300.19 cells and cTfR-expressing 300.19 cells were determined by flow cytometry. 300.19 is a murine pre-B cell line, originating from a lymphoma, and was stably transfected by nucleoporation with a plasmid expressing either hTfR or cTfR (SEQ ID NO: 1 and SEQ ID NO: 2, respectively).

300.19 TFRC-expressing cells were coated at 5×10⁵ cells/well on 96-well High Bind plate (MSD L15XB-3) and 100 μL/well of anti-TfR-GAA molecule were added and incubated for 45 min at 4° C. and washed three times with PBS 1% BSA. 100 μL/well of goat anti-human IgG conjugated with Alexa488 (Jackson ImmunoResearch, #109-545-098) was added and incubated for 45 min at 4° C. and washed three times with PBS 1% BSA. Antibody binding was evaluated after centrifugation and resuspension of cells by adding 200 μl/well PBS 1% BSA and read using Guava® easyCyte™ 8HT Flow Cytometry System. EC50 values were estimated using BIOST@T-BINDING and reported in Table 6.

TABLE 6
Characterization of GAA-Loaded TfR Binders

| | | | | 531v25Fab- | |
|---|---|---|---|---|---|
| | | 531v25Fab- | 531v25Fab- | FcOL-LC- | 531v25mAb- |
| Characteristics | GAA | LC-GAA | FcOL-GAA | GAA | GAA |
| MW (kDa) | 110 | 149 | 198 | 198 | 244 |
| GAA Enzymatic | 100% | 112% | 122% | 92% | 71% |
| activity | | | | | |
| (4-MU fluorometric, | | | | | |
| % GAA) | | | | | |
| Binding human TfR | ND | 9.9 | 19.5 | 21 | 2.7 |
| EC₅₀ in nM (FACS) | | | | | |
| Binding cynomolgus | ND | 74.6 | ≥132 | 107 | 2.6 |
| TfR | | | | | |
| EC₅₀ in nM (FACS) | | | | | |
| Cynomolgus/human | ND | 7.5 | ≥6.8 | 5.1 | 1 |
| ratio (TfR) | | | | | |

Affinity of 531v25Fab-FcOL-LC-GAA and 531v25Fab-FcOL-GAA (SPR) was assessed as in example 3A, and reported in Table 7. Affinity by SPR was also measured for the 531v25-mAb-GAAx2 molecule (a GAA sequence fused to both HCs).

These data show that the affinity features as well as the cynomolgus/human ratio of the 2 (monovalent) 531v25 Fab-FcOL molecules is conserved in the fusion constructs with GAA. The FACS data also confirmed that the 531v25 antibody is able to bind to the hTfR and cTfR in their native conformation. The data obtained with the 531v25-mAb-GAAx2 molecule show both affinity and avidity for TfR binding, as a consequence of the two TfR binding sites.

TABLE 7
TfR-binding affinity for 531v25Fab-FcOL-GAA, 531v25Fab-
FcOL-LC-GAA and 531v25-mAb-GAAx2 by SPR

| | | hTfR | cTfR | |
|---|---|---|---|---|
| | | K_{D} | K_{D} | Cynomolgus/ |
| Format | Analyte | (nM) | (nM) | human |
| Monovalent | 531v25Fab-FcOL-GAA | 27.2 | 89 | 3.3 |
| Monovalent | 531v25Fab-FcOL-LC-GAA | 6.5 | 154.1 | 23.7 |
| Bivalent | 531v25-mAb-GAAx2 | 1.8 | 7.6 | / |

We also sought to assess the affinity by BLI of the 531v25Fab-FcOL-GAA and to compare it with 531v25-Fab-FcOL (i.e., without GAA). Affinity by BLI of 531v25Fab-FcOL-GAA was assessed as in Example 3A and the data are reported in Table 8. These data show that the affinity features as well as the cynomolgus/human ratio of the 531v25 molecule is conserved in the fusion construct with GAA.

TABLE 8
Comparison of TfR affinity of 531v25Fab-
FcOL-GAA and 531v25Fab-FcOL by BLI

| | hTfR | cTfR | Cynomolgus/ |
|---|---|---|---|
| | K_{D} (nM) | K_{D} (nM) | human |
| 531v25-Fab-FcOL | 22.1 | 103.4 | 4.7 |
| 531v25Fab-FcOL-GAA | 17.5 | 69.9 | 4.0 |

B. No Competition for Human TfR Binding Observed with Transferrin from 531v25-Fab-FcOL-GAA as Determined by BLI

We sought to confirm absence of binding competition to TfR with transferrin by BLI with saturation either by holoTF (holo-transferrin) or 531v25Fab-FcOL-GAA. This was assayed as in Example 3B and the data are reported in Table 9.

The data show that transferrin binding was not affected by pre-association of 531v25Fab-FcOL-GAA. Holo transferrin saturation to human TfR only minimally affected 531v25Fab-FcOL-GAA binding to hTfR. These data indicate that there is no competition between 531v25Fab-FcOL-GAA and transferrin for binding to the transferrin receptor.

TABLE 9
Competition with Transferrin for hTfR binding by BLI

| Analyte | K_{D} (nM) |
|---|---|
| 531v25Fab-FcOL-GAA | 17.5 |
| 500 nM holoTf saturation, then 531v25-Fab-FcOL-GAA | 30 |
| Holo-TF | 1.2 |
| 500 nM 531v25-Fab-FcOL-GAA saturation, then holoTF | 1.0 |

### Example 6: Assessment of 531v25-GAA Fusion Molecules in Mice

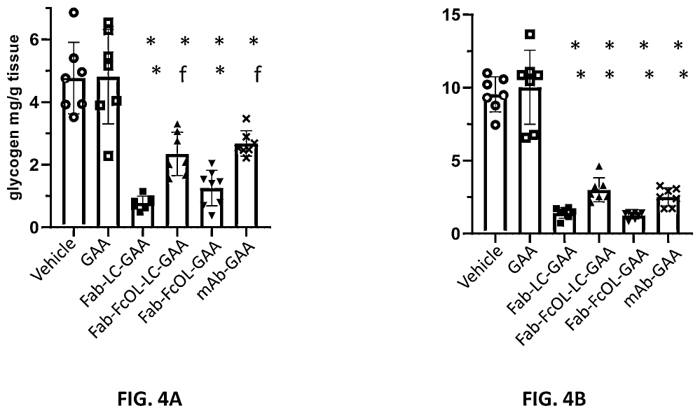

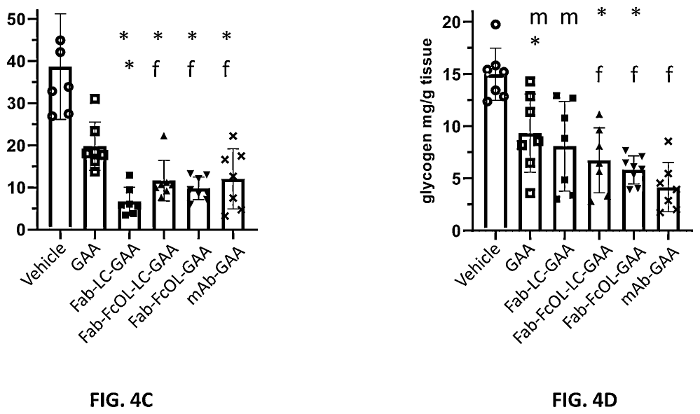

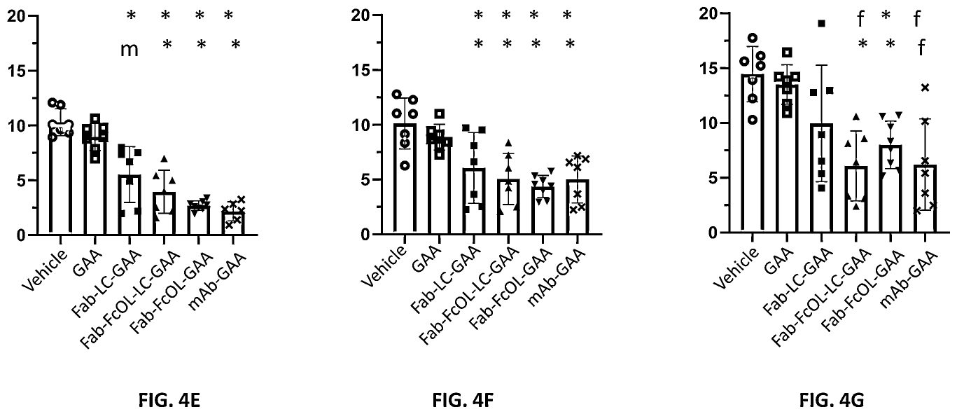

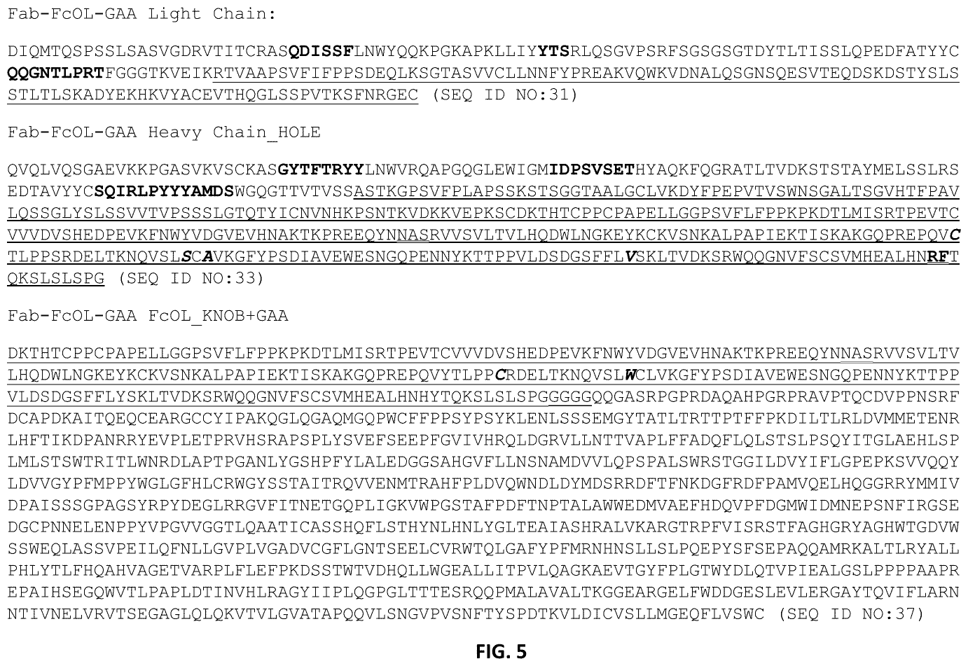

Next, we compared the four anti-TfR-GAA molecules in a mouse model for Pompe disease, which is knocked-out for the endogenous GAA gene and knocked-in for the ectodomain of the human TfR gene. Glycogen levels in various organs (brain, spinal cord, heart and four muscles) were assessed following administration of the anti-TfR-GAA molecules in mice (FIGS. 4A-G and Table 10). Anti-TfR-GAA molecules assessed—531v25mAb-GAA, 531v25Fab-LC-GAA, 531v25Fab-FcOL-GAA and 531v25Fab-FcOL-LC-GAA—were as detailed above. A vehicle and GAA were used as control.

Pompe mice (GAA-KO 6neo/6neo, GAAtm1 Rabn) (Raben et al., J Biol Chem. (1998) 273:19086-92) were crossed to become double homozygous for both alleles with a humanized TfR knock-in (KI) mice (hTfR1-KI mice). The hTfR1-KI mice [C57BL/6-TfR-tm2618(TfR)Arte (Taconic)] express a chimeric TfR made of the human extracellular domain fused with the murine transmembrane and cytoplasmic domains (exons 4-19 of the murine TfR were replaced by the corresponding human sequence in C57BL/6 NTAc ES cells without disrupting the murine 3′ untranslated region). Expression is driven from the endogenous murine TfRI promoter to comparable levels in all tissues compared to TfRI in wildtype mice as determined by qPCR and whole tissue immunoblots.

hTfR-KI-Pompe mice, 9-10 months of age, were administered 4 weekly doses of 180 nmol/kg test article. This is molar equivalent to 20 mg/kg alglucosidase alfa (GAA). Each test group contained 7 mice/treatment (3M, 4F). Antihistamine diphenhydramine was administered to all animals starting with the second dose to mitigate hypersensitivity responses to the administration of a human protein. Animals were euthanized 7 days post last dose, perfused with PBS and target tissues were collected. Tissues were homogenized at 4° C. at 1:10 or 1:50 volume/weight excess of water/tissue at using a beads disruptor. Glycogen content was quantified biochemically using a commercial colorimetric/fluorometric kit (BioVision, Milpitas, CA), following manufacturer's instructions. Non-hydrolysis enzyme treated samples were used for each sample to correct for glucose background. All values were back calculated to mg glycogen/gr initial tissue.

Alglucosidase alfa (GAA), which does not cross the blood brain barrier, resulted in no change to glycogen levels in the brain and spinal cord compared to vehicle controls in hTfR-KI-Pompe mice. By contrast, treatment with all anti-TfR-GAA constructs resulted in significant glycogen clearance in the CNS (FIGS. 4A and 4B). The percent reduction is calculated vis-à-vis vehicle treatment or GAA treatment as shown in Table 10 below.

The data show that both 531v25Fab-LC-GAA and 531v25Fab-FcOL-GAA were most effective in the brain, with 84% and 74% glycogen reduction, respectively. For heart muscle, all anti-TfR targeted GAA constructs were more efficacious in lowering tissue glycogen, with Fab-LC-GAA and Fab-FcOL-GAA showing superior effects (FIG. 4C and Table 10). In all skeletal muscles, anti-TfR-GAA constructs with Fc domain seemed more potent than Fab-LC-GAA, with mAb-GAA equal or better than Fab-FcOL-GAA (FIGS. 4D-G and Table 10).

TABLE 10
Glycogen Levels in Mouse Model of Pompe Disease

| | Brain | | Spinal cord | | Heart | | Diaphragm | | Gastrocnemius | | Quadriceps | | Triceps | |
|---|---|---|---|---|---|---|---|---|---|---|---|---|---|---|
| | vs | vs | vs | vs | vs | vs | vs | vs | vs | vs | vs | vs | vs | vs |
| Comparison | Veh | GAA | Veh | GAA | Veh | GAA | Veh | GAA | Veh | GAA | Veh | GAA | Veh | GAA |
| Vehicle | 100% | | 100% | | 100% | | 100% | | 100% | | 100% | | 100% | |
| GAA | 1% | 100% | 5% | 100% | −49% | 100% | −38% | 100% | −13% | 100% | −12% | 100% | −7% | 100% |
| 531v25Fab-LC-GAA | −84% | −84% | −85% | −86% | −83% | −66% | −46% | −13% | −46% | −38% | −40% | −32% | −31% | −26% |
| 531v25Fab-FcOL-LC-GAA | −51% | −51% | −69% | −70% | −70% | −41% | −55% | −28% | −62% | −56% | −50% | −43% | −58% | −55% |
| 531v25Fab-FcOL-GAA | −74% | −74% | −87% | −88% | −75% | −50% | −61% | −38% | −74% | −70% | −57% | −51% | −45% | −41% |
| 531v25mAb-GAA | −44% | −44% | −74% | −75% | −69% | −39% | −72% | −55% | −79% | −76% | −50% | −43% | −57% | −54% |

*Veh: vehicle.

### Example 7: Pharmacokinetics (PK) of Anti-TfR-GAA Molecules in Human TfR-Knock-In Mice

Next, we sought to determine the behaviour of various anti-TfR-GAA molecules in mice. The following molecules were used in this study: 531v25mAb-GAA, 531v25Fab-GAA, 531v25Fab-FcOL-GAA, and 531v25Fab-FcOL-LC-GAA.

Experiments were performed in hTfR-KI mice (see Example 6). All mice were treatment-naïve males and females between the ages of 4 and 6 months at study start. For dosing, the anti-TfR-GAA molecules were prepared in 10 mM histidine (pH 6), 150 mM NaCl formulation buffer and administered as single intravenous doses of 70 nmol/kg into the tail vein with a dose volume of 10 mL/kg. A total of 3 animals per sampling times were evaluated for each compound utilizing a terminal sampling approach (0.25, 2, 5, 24, 48, 72 and 168 hours) across the study duration of 7 days. Blood, quadriceps, and brain samples were collected by sampling time. Blood samples were centrifuged at 4° C. for 10 minutes at 1500 g and isolated plasma was stored at −80° C. until analysis. Before collection, brains were flushed in situ with a saline solution to avoid blood contamination and then homogenized in lysis buffer (1% NP-40). Brain and quadriceps were frozen at −80° C. immediately after collection.

After sampling of tissues, 5 volumes of lysis buffer were added in tubes containing ceramic beads for brain hemisphere and metallic beads for quadriceps. The homogenization was realized with Precellys®, one cycle of 2 times at 5500 rpm for 20 sec with a 10 sec pause for brain and 2 cycles of 4 times at 7500 rpm for 20 sec with a 10 sec pause for quadriceps. After a centrifugation at 1400 rpm for 30 sec at 4ºC, the tubes were placed on a roller for 1 hour at 4ºC and aliquoted at 100 μL volume in low binding tubes and stored at −80° C. until analysis. The concentration of each anti-TfR-GAA molecule at each time point was determined by an immunoassay method using MSD platform (QuickPlex SQ120). The assay was based on the GAA recognition properties of rabbit anti-GAA antibody coated on microtiter plate (Standard MSD 96 wells sector plate) (GAA-capture) and the use of the goat anti-mouse kappa ruthenium tracer for the detection by electrochemiluminescence (Fab kappa LC-detection). Samples (standards, quality controls and study samples) were diluted 10-fold in PBS-Tween 0.1% BSA buffer and dispensed in a 96-well microtiter plate. All analyses were performed in duplicate, and the range of quantification was 0.0078 to 1000 ng/mL.

PK parameters of anti-TfR-GAA molecules in hTfR-KI mice are summarized in Tables 11, 12, and 13.

531v25Fab-GAA exhibited the highest clearance compared to the other constructs. There was rapid elimination of all constructs from plasma, with 531v25Fab-GAA having the shortest half-life (t_{1/2}) (Table 11). In the brain, the highest exposure was observed for the two 531v25Fab-FcOL constructs with a Brain/Plasma AUC=4%. The shortest elimination half-life from the brain was observed with the 531v25Fab-GAA construct (Table 12).

TABLE 11
Single-Dose PK in hTfR-KI mice (plasma)

| | | 531v25Fab- | | |
|---|---|---|---|---|
| | 531v25Fab- | FcOL-LC- | 531v25Fab- | 531v25mAb- |
| Plasma PK | GAA | GAA | FcOL-GAA | GAA |
| C₀ or Cₘₐₓ | 610 | 1960 | 1610 | 3140 |
| (nmol/L) | | | | |
| AUC | 368 | 1480 | 2690 | 2660 |
| (nmol*h/L) | | | | |
| Cl | 182 | 47.8 | 26.3 | 26.2 |
| (mL/h/kg) | | | | |
| Vss | 43.5 | 61.5 | 67 | 14.7 |
| (mL/kg) | | | | |
| t_{1/2z} | 1.6 | 26.3 | 12.5 | 3.8 |
| (h) | | | | |

Finally, the highest exposure in quadriceps was observed for the two 531v25Fab-FcOL constructs (Table 12). The shortest elimination half-life was observed with the 531v25Fab-GAA (Table 13).

TABLE 12
Single-Dose PK in hTfR-KI mice (quadriceps)

| | | 531v25Fab- | | |
|---|---|---|---|---|
| Quadriceps | 531v25Fab- | FcOL-LC- | 531v25Fab- | 531v25mAb- |
| PK | GAA | GAA | FcOL-GAA | GAA |
| C₀ or Cₘₐₓ | 0.9 | 4.3 | 3.5 | 1.4 |
| (nmol/L) | | | | |
| AUC | 1.5 | 48.9 | 45.8 | 19.1 |
| (nmol*h/L) | | | | |
| t_{1/2z} | 1.5 | 97.6 | 54 | 17.7 |
| (h) | | | | |

TABLE 13
Single-Dose PK in hTfR-KI mice (brain)

| | | 531v25Fab- | | |
|---|---|---|---|---|
| | 531v25Fab- | FcOL-LC- | 531v25Fab- | 531v25mAb- |
| Brain PK | GAA | GAA | FcOL-GAA | GAA |
| C₀ or Cₘₐₓ | 6.8 | 9.6 | 9.3 | 3.8 |
| (nmol/L) | | | | |
| AUC | 42.4 | 85.4 | 116 | 78 |
| (nmol*h/L) | | | | |
| t_{1/2z} | 6.6 | 16.6 | 16.4 | 16.9 |
| (h) | | | | |

Altogether, these results show that both 531v25Fab-FcOL-LC-GAA and 531v25Fab-FcOL-GAA molecules exhibit the most favourable PK behaviour compared to 531v25Fab-GAA and 531v25mAb-GAA, with the highest plasma exposure, the lowest clearance, and the highest distribution in brain and quadriceps.

### Example 8: MALAT1 mRNA Knockdown Using Anti-hTfR 531v25-Fab-FcOL-Anti-MALAT-1 ASO Oligonucleotide Conjugate in hTfR-KI Mice

To evaluate if additional moieties could be efficiently targeted to the brain and skeletal muscle, we assessed ASO-mediated knockdown of MALAT1, a nuclear RNA expressed in most tissues. For this study the following reagents were prepared.

531v25 Fab-FcOL (consisting of SEQ ID NOs: 31, 34, and 56) was transiently expressed in Expi293 cells, then purified with a protein A capture step and a size exclusion chromatography polishing step. SEQ ID NO: 56 is similar to SEQ ID NO: 33, but with an A129C (by IMGT numbering; A114C by Kabat numbering) mutation (THIOMAB™ technology) for site-specific conjugation of the MALAT1 ASO. To generate the antibody-ASO conjugate (anti-hTfR-MALAT1-ASO conjugate), the 531v25 Fab-FcOL was reduced with 50× molar excess TCEP for 3 hrs at room temperature, followed by desalting over a HiPrep 26/10 desalting column. The sample was allowed to re-oxidize overnight, then mixed with 3 molar excess mouse MALAT1-ASO (GCATTCTAATAGCAGC; SEQ ID NO: 57) with an SMCC linker. The MALAT1-ASO sequence is shown below with modifications:

5′-(SMCC)(NHC6)GbsCbsAbsdTsdTs(5MdC)sdTsdAsdAsdTsdAsdGs(5MdC)sAbsGbsCb-3′
Nb: LNA residues (including LNA-5MeC and LNA T/LNA-5MeU)
dN: DNA residues
(5MdC): 5-Methyl DNA C
s: phosphorothioate backbone modification
(NHC6): aminohexyl linker
(SMCC): succinimidyl-trans-4-(N-maleimidylmethyl)cyclohexane-1-carboxylate

The conjugation progress was monitored with SDS-PAGE gel until completion, then the conjugate complex was purified from excess unreacted ASO using size exclusion chromatography.

To generate ASO alone, maleimide in the SMCC linker was inactivated with 10 mM cysteine for 30 min at room temperature.

To evaluate tissue-specific knockdown of MALAT1 mRNA, hTfR-KI mice were dosed four times (twice a week at 3-4 day dosing intervals) with 400 nmol/kg inactivated SMCC-MALAT1 ASO (N=5), or 400 nmol/kg molar equivalent of ASO in the form of the anti-hTfR-MALAT1-ASO conjugate (DAR1) (N=3) or saline vehicle (N=5). Three days (72 hrs) following the fourth dose, mice were anesthetized with ketamine/xylazine and transcardially perfused with heparinated DBPS with Ca/Mg. Tissues (brain, heart, gastrocnemius, quadriceps, spleen and sciatic nerve) were harvested and weighted for quantitative PCR for MALAT1, with B-Actin acting as a house-keeping gene. RNA was isolated from frozen tissue samples. Tissue was homogenized using a bead mill homogenizer in TRIzol/chloroform in 2 mL tubes containing 2.8 mm ceramic beads. Subsequently, RNA was isolated following Qiagen's RNeasy 96 QIAcube HT kit instructions. cDNA was generated using Applied Biosystems™ High-Capacity cDNA Reverse Transcription Kit with RNase Inhibitor. Finally, qPCR was run on a QuantStudio™ 7 Flex, using PrimeTime® Gene Expression Master Mix (IDT) and the following TaqMan® qPCR primer sets: ß-actin forward 5′-GTACGACCAGAGGCATACAG-3′ (SEQ ID NO: 58); reverse 5′-ACCGTGAAAAGATGACCCAG-3′ (SEQ ID NO: 59) Probe /5HEX/ACCTTCAAC/ZEN/ACCCCAGCCATGTA/3IABKFQ/(ACCTTCAAC: SEQ ID NO: 60; ACCCCAGCCATGTA: SEQ ID NO: 61); mouse MALAT1: MALAT1 Mm01227912 s1 FAM-MGB mouse from ThermoFisher TaqMan® MGB Probes.

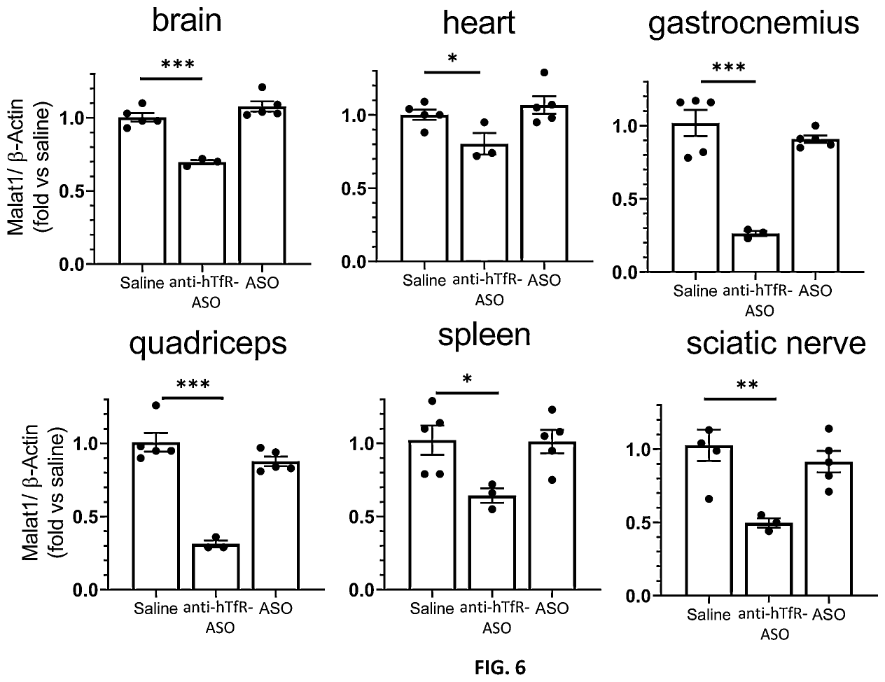

Our data show that anti-hTfR-MALAT1-ASO conjugate significantly knocks down MALAT1 mRNA in brain, heart, gastrocnemius, and quadriceps as well as spleen and sciatic nerve, compared to equal molar amounts of intravenously dosed free MALAT1-ASO or vehicle treated mice. Significance was determined using one-way ANOVA with Dunnet's multiple comparisons (GraphPad v9.5.0) (*=p<0.05, **=p<0.005, *** p<0.0001, vs. vehicle) (FIG. 6).

These findings demonstrate that 531v25 Fab-FcOL can be used to target oligonucleotides such as MALAT1-ASO for gene knockdown in brain, sciatic nerve and skeletal muscle tissues in vivo. These data provide evidence that a range of moieties (including enzymes and oligonucleotides), when conjugated to our 531v25-anti-TfR moiety, can be efficiently targeted to brain and muscle cells with functional effects such as replenishing enzyme function (GAA) or reducing gene expression levels (MALAT1).

### Sequences

Sequences described in the present disclosure are summarized in the table below (SEQ: SEQ ID NO).

| | | |
|---|---|---|
| SEQ | Description | Sequence |

Full-Length Transferrin Receptor (TfR)

| | | |
|---|---|---|
| 1 | Human TfR | MMDQARSAFSNLEGGEPLSYTRESLARQVDGDNSHVEMKLAVDEEE |
| | | NADNNTKANVTKPKRCSGSICYGTIAVIVFFLIGFMIGYLGYCKGV |
| | | EPKTECERLAGTESPVREEPGEDFPAARRLYWDDLKRKLSEKLDST |
| | | DETGTIKLLNENSYVPREAGSQKDENLALYVENQFREFKLSKVWRD |
| | | QHFVKIQVKDSAQNSVIIVDKNGRLVYLVENPGGYVAYSKAATVTG |
| | | KLVHANFGTKKDFEDLYTPVNGSIVIVRAGKITFAEKVANAESLNA |
| | | IGVLIYMDQTKFPIVNAELSFFGHAHLGTGDPYTPGFPSENHTQFP |
| | | PSRSSGLPNIPVQTISRAAAEKLFGNMEGDCPSDWKTDSTCRMVTS |
| | | ESKNVKLTVSNVLKEIKILNIFGVIKGFVEPDHYVVVGAQRDAWGP |
| | | GAAKSGVGTALLLKLAQMFSDMVLKDGFQPSRSIIFASWSAGDEGS |
| | | VGATEWLEGYLSSLHLKAFTYINLDKAVLGTSNFKVSASPLLYTLI |
| | | EKTMQNVKHPVTGQFLYQDSNWASKVEKLTLDNAAFPFLAYSGIPA |
| | | VSFCFCEDTDYPYLGTTMDTYKELIERIPELNKVARAAAEVAGQFV |
| | | IKLTHDVELNLDYERYNSQLLSFVRDLNQYRADIKEMGLSLOWLYS |
| | | ARGDFFRATSRLTTDFGNAEKTDRFVMKKLNDRVMRVEYHELSPYV |
| | | SPKESPFRHVFWGSGSHTLPALLENLKLRKONNGAFNETLERNQLA |
| | | LATWTIQGAANALSGDVWDIDNEF |
| 2 | Cynomolgus monkey | MMDQARSAFSNLEGGEPLSYTRESLARQVDGDNSHVEMKLGVDEEE |
| | (Macaca fascicularis) | NTDNNTKANGTKPKRCGGNICYGTIAVIIFFLIGEMIGYLGYCKGV |
| | TfR | EPKTECERLAGTESPAREEPEEDFPAAPRLYWDDLKRKLSEKLDTT |
| | | DFTSTIKLLNENLYVPREAGSQKDENLALYIENQFREFKLSKVWRD |
| | | QHFVKIQVKDSAQNSVIIVDKNGGLVYLVENPGGYVAYSKAATVTG |
| | | KLVHANFGTKKDFEDLDSPVNGSIVIVRAGKITFAEKVANAESLNA |
| | | IGVLIYMDQTKFPIVKADLSFFGHAHLGTGDPYTPGFPSENHTQFP |
| | | PSQSSGLPNIPVQTISRAAAEKLFGNMEGDCPSDWKTDSTCKMVTS |
| | | ENKSVKLTVSNVLKETKILNIFGVIKGFVEPDHYVVVGAQRDAWGP |
| | | GAAKSSVGTALLLKLAQMESDMVLKDGFQPSRSIIFASWSAGDEGS |
| | | VGATEWLEGYLSSLHLKAFTYINLDKAVLGTSNFKVSASPLLYTLI |
| | | EKTMQDVKHPVTGRSLYQDSNWASKVEKLTLDNAAFPFLAYSGIPA |
| | | VSFCFCEDTDYPYLGTTMDTYKELVERIPELNKVARAAAEVAGQFV |
| | | IKLTHDTELNLDYERYNSQLLLFLRDLNQYRADVKEMGLSLOWLYS |
| | | ARGDFFRATSRLTTDERNAEKRDKFVMKKLNDRVMRVEYYFLSPYV |
| | | SPKESPFRHVFWGSGSHTLSALLESLKLRRONNSAFNETLERNQLA |
| | | LATWTIQGAANALSGDVWDIDNEF |
| 3 | Mouse | MMDQARSAFSNLEGGEPLSYTRESLARQVDGDNSHVEMKLAADEEE |
| | (Mus musculus) | NADNNMKASVRKPKRENGRLCFAAIALVIFFLIGEMSGYLGYCKRV |
| | TfR | EQKEECVKLAETEETDKSETMETEDVPTSSRLYWADLKTLLSEKLN |
| | (NP_035768.1) | SIEFADTIKQLSQNTYTPREAGSQKDESLAYYIENQFHEFKESKVW |
| | | RDEHYVKIQVKSSIGQNMVTIVOSNGNLDPVESPEGYVAFSKPTEV |
| | | SGKLVHANFGTKKDFEELSYSVNGSLVIVRAGEITFAEKVANAQSE |
| | | NAIGVLIYMDKNKFPVVEADLALFGHAHLGTGDPYTPGFPSENHTQ |
| | | FPPSQSSGLPNIPVQTISRAAAEKLFGKMEGSCPARWNIDSSCKLE |
| | | LSQNQNVKLIVKNVLKERRILNIFGVIKGYEEPDRYVVVGAQRDAL |
| | | GAGVAAKSSVGTGLLLKLAQVESDMISKDGFRPSRSIIFASWTAGD |
| | | FGAVGATEWLEGYLSSLHLKAFTYINLDKVVLGTSNFKVSASPLLY |
| | | TLMGKIMQDVKHPVDGKSLYRDSNWISKVEKLSEDNAAYPFLAYSG |
| | | IPAVSFCFCEDADYPYLGTRLDTYEALTQKVPQLNQMVRTAAEVAG |
| | | QLIIKLTHDVELNLDYEMYNSKLLSFMKDLNQFKTDIRDMGLSLOW |
| | | LYSARGDYFRATSRLTTDFHNAEKTNRFVMREINDRIMKVEYHELS |
| | | PYVSPRESPFRHIFWGSGSHTLSALVENLKLRQKNITAFNETLERN |
| | | QLALATWTIQGVANALSGDIWNIDNEF |

Mouse Anti-human TfR 531 (Parental)

| | | |
|---|---|---|
| 4 | 531-1L2_mouse V_{H} (CDRs in box) | 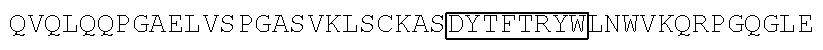 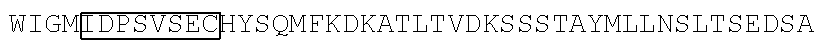 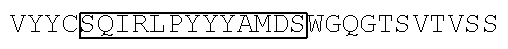 |
| 5 | 531-HCDR1 | DYTFTRYW |
| | (IMGT) | |
| 6 | 531-1L2 HCDR2 | IDPSVSEC |
| | (IMGT) | |
| 7 | 531-1L2_HCDR3 | SQIRLPYYYAMDS |
| | (IMGT) | |
| 8 | 531-1L2 mouse V_{L} (CDRs in box) | 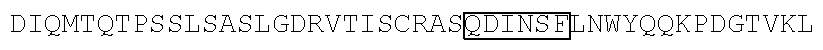 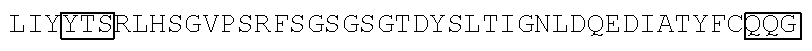 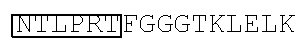 |
| 9 | 531-1L2_LCDR1 | QDINSF |
| | (IMGT) | |
| 10 | 531-1L2_LCDR2 | YTS |
| | (IMGT) | |
| 11 | 531-1L2_LCDR3 | QQGNTLPRT |
| | (IMGT) | |

Human Germline for Humanization

| | | |
|---|---|---|
| 12 | IGHV_23_05 | EVQLLESGGGLVQPGGSLRLSCAASGFTFSSYAMSWVRQAPGKGLE |
| | germline | WVSAIYSSGSSTYYADSVKGRFTISRDNSKNTLYLQMNSLRAEDTA |
| | | VYYCAK |
| 13 | IGHV1_46_01 | QVQLVQSGAEVKKPGASVKVSCKASGYTFTSYYMHWVRQAPGQGLE |
| | germline | WMGIINPSGGSTSYAQKFQGRVTMTRDTSTSTVYMELSSLRSEDTA |
| | | VYYCAR |
| 14 | IGHJ6_01 | YYYYYGMDVWGQGTTVTVSS |
| | germline | |
| 15 | IGKV1_39_01 | DIQMTQSPSSLSASVGDRVTITCRASQSISSYLNWYQQKPGKAPKL |
| | germline | LIYAASSLQSGVPSRESGSGSGTDFTLTISSLOPEDFATYYCQQSY |
| | | STP |
| 16 | IGKJ4_01 | LTFGGGTKVEIK |
| | germline | |

Humanized V_{H} and V_{L} Variants

| | | |
|---|---|---|
| 17 | 531 v1 V_{H} (CDRs in box) | 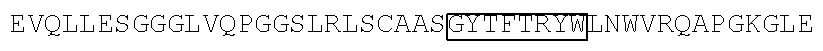 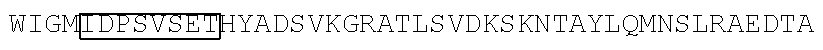 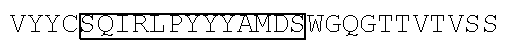 |
| 18 | 531 v2 V_{H} (CDRs in box) | 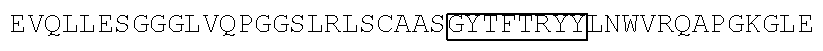 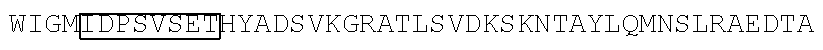 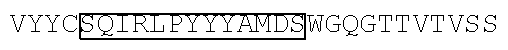 |
| 19 | 531 v3 V_{H} (VH2A) (CDRs in box) | 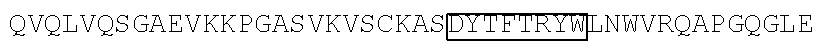 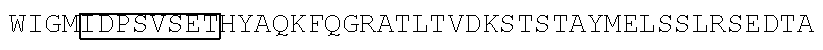 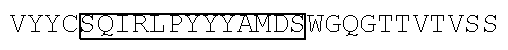 |
| 20 | 531 v4 V_{H} (VH2B) (CDRs in box) | 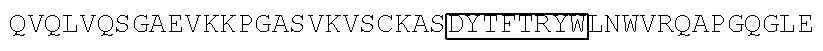 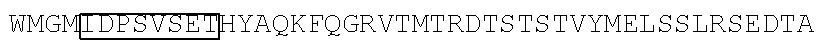 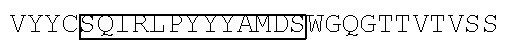 |
| 21 | 531 v5 V_{H} (VH2A_motifs4) [V_{H} used for 531v25] (CDRs in box) | 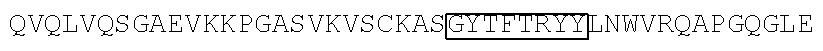 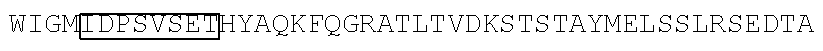 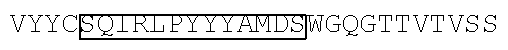 |
| 22 | 531 v1 V_{L} (VL3A) (CDRs in box) | 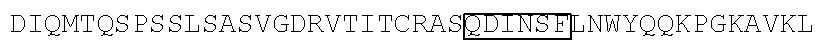 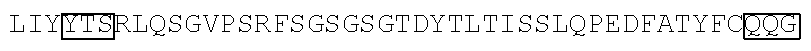 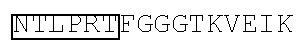 |
| 23 | 531 v2 V_{L} (VL3A_motifs1) (CDRs in box) | 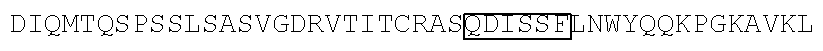 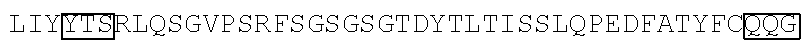 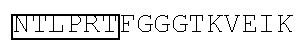 |
| 24 | 531 v3 V_{L} (VL3B_motifs1) (CDRs in box) | 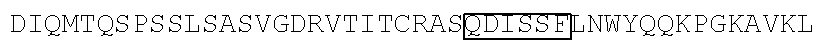 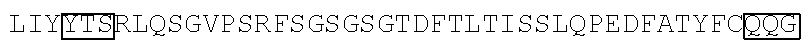 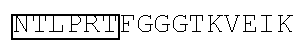 |
| 25 | 531 v4 V_{L} (VL3C_motifs1) [V_{L} used for 531v25] (CDRs in box) | 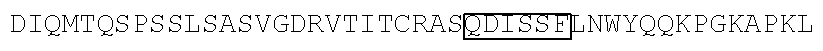 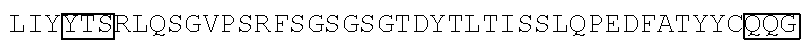 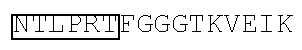 |

Humanized Antibodies and Antigen-Binding Fragments

| | | |
|---|---|---|
| 26 | 531v25 HCDR1 | GYTFTRYY |
| | (IMGT) | |
| 27 | 531 vhz1 HCDR1 | GYTFTRYW |
| | (IMGT) | |
| 5 | 531 vhz2 HCDR1 | DYTFTRYW |
| | (IMGT) | |
| 28 | 531v25 HCDR2 | IDPSVSET |
| | (IMGT) | |
| 7 | 531v25 HCDR3 | SQIRLPYYYAMDS |
| | IMGT | |
| 29 | 531v25 LCDR1 | QDISSE |
| | (IMGT) | |
| 9 | 531 vhz1 LCDR1 | QDINSF |
| | (IMGT) | |
| 10 | 531v25 LCDR2 | YTS |
| | (IMGT) | |
| 11 | 531v25 LCDR3 | QQGNTLPRT |
| | (IMGT) | |

531v25 Constructs (without GAA)

| | | |
|---|---|---|
| 30 | 531v25 Fab | QVQLVQSGAEVKKPGASVKVSCKASGYTFTRYYLNWVRQAPGQGLE |
| | HC | WIGMIDPSVSETHYAQKFQGRATLTVDKSTSTAYMELSSLRSEDTA |
| | | VYYCSQIRLPYYYAMDSWGQGTTVTVSSASTKGPSVFPLAPSSKST |
| | | SGGTAALGCLVKDYFPEPVTVSWNSGALTSGVHTFPAVLOSS |
| | | GLYSLSSVVTVPSSSLGTQTYICNVNHKPSNTKVDKKVEPKSC |
| 31 | 531v25 Fab | DIQMTQSPSSLSASVGDRVTITCRASQDISSFLNWYQQKPGKAPKL |
| | LC | LIYYTSRLQSGVPSRFSGSGSGTDYTLTISSLOPEDFATYYCQQGN |
| | (Kappa) | TLPRTFGGGTKVEIKRTVAAPSVFIFPPSDEQLKSGTASVVCLLNN |
| | | FYPREAKVQWKVDNALQSGNSQESVTEQDSKDSTYSLSSTLT |
| | | LSKADYEKHKVYACEVTHQGLSSPVTKSENRGEC |
| 32 | 531v25 mAb | QVQLVQSGAEVKKPGASVKVSCKASGYTFTRYYLNWVRQAPGOGLE |
| | HC | WIGMIDPSVSETHYAQKFQGRATLTVDKSTSTAYMELSSLRSEDTA |
| | (IgG1, with NNAS | VYYCSQIRLPYYYAMDSWGQGTTVTVSSASTKGPSVFPLAPSSKST |
| | mutations) | SGGTAALGCLVKDYFPEPVTVSWNSGALTSGVHTFPAVLQSS |
| | | GLYSLSSVVTVPSSSLGTQTYICNVNHKPSNTKVDKKVEPKSCDKT |
| | | HTCPPCPAPELLGGPSVFLFPPKPKDTLMISRTPEVTCVVVDVSHE |
| | | DPEVKFNWYVDGVEVHNAKTKPREEQYNNASRVVSVLTVLHQDWLN |
| | | GKEYKCKVSNKALPAPIEKTISKAKGQPREPQVYTLPPSRDE |
| | | LTKNQVSLTCLVKGFYPSDIAVEWESNGQPENNYKTTPPVLDSDGS |
| | | FFLYSKLTVDKSRWQQGNVFSCSVMHEALHNHYTQKSLSLSPG |
| 31 | 531v25 mAb | DIQMTQSPSSLSASVGDRVTITCRASQDISSELNWYQQKPGKAPKL |
| | LC | LIYYTSRLQSGVPSRFSGSGSGTDYTLTISSLOPEDFATYYCQQGN |
| | | TLPRTFGGGTKVEIKRTVAAPSVFIFPPSDEQLKSGTASVVCLLNN |
| | | FYPREAKVQWKVDNALQSGNSQESVTEQDSKDSTYSLSSTLT |
| | | LSKADYEKHKVYACEVTHQGLSSPVTKSENRGEC |
| 31 | 531v25 Fab-FcOL | DIQMTQSPSSLSASVGDRVTITCRASQDISSELNWYQQKPGKAPKL |
| | LC | LIYYTSRLQSGVPSRFSGSGSGTDYTLTISSLOPEDFATYYCQQGN |
| | | TLPRTFGGGTKVEIKRTVAAPSVFIFPPSDEQLKSGTASVVCLLNN |
| | | FYPREAKVQWKVDNALQSGNSQESVTEQDSKDSTYSLSSTLT |
| | | LSKADYEKHKVYACEVTHQGLSSPVTKSENRGEC |
| 33 | 531v25 Fab-FcOL | QVQLVQSGAEVKKPGASVKVSCKASGYTFTRYYLNWVRQAPGQGLE |
| | HC | WIGMIDPSVSETHYAQKFQGRATLTVDKSTSTAYMELSSLRSEDTA |
| | (IgG1, with NNAS, | VYYCSQIRLPYYYAMDSWGQGTTVTVSSASTKGPSVFPLAPSSKST |
| | hole, and RF | SGGTAALGCLVKDYFPEPVTVSWNSGALTSGVHTFPAVLQSS |
| | mutations) | GLYSLSSVVTVPSSSLGTQTYICNVNHKPSNTKVDKKVEPKSCDKT |
| | | HTCPPCPAPELLGGPSVFLFPPKPKDTLMISRTPEVTCVVVDVSHE |
| | | DPEVKFNWYVDGVEVHNAKTKPREEQYNNASRVVSVLTVLHQDWLN |
| | | GKEYKCKVSNKALPAPIEKTISKAKGQPREPQVCTLPPSRDE |
| | | LTKNQVSLSCAVKGFYPSDIAVEWESNGQPENNYKTTPPVLDSDGS |
| | | FFLVSKLTVDKSRWQQGNVFSCSVMHEALHNRFTQKSLSLSPG |
| 34 | 531v25 Fab-FcOL | DKTHTCPPCPAPELLGGPSVFLFPPKPKDTLMISRTPEVTCVVVDV |
| | Fc Polypeptide | SHEDPEVKENWYVDGVEVHNAKTKPREEQYNNASRVVSVLTVLHQD |
| | (IgG1, with knob | WLNGKEYKCKVSNKALPAPIEKTISKAKGQPREPQVYTLPPCRDEL |
| | mutations) | TKNQVSLWCLVKGFYPSDIAVEWESNGQPENNYKTTPPVLDS |
| | | DGSFFLYSKLTVDKSRWQQGNVFSCSVMHEALHNHYTQKSLSLSPG |

531v25-GAA Constructs

| | | |
|---|---|---|
| 35 | Exemplary human | QQGASRPGPRDAQAHPGRPRAVPTQCDVPPNSREDCAPDKAITQEQ |
| | GAA | CEARGCCYIPAKOGLQGAQMGQPWCFFPPSYPSYKLENLSSSEMGY |
| | (alglucosidase alfa) | TATLTRTTPTFFPKDILTLRLDVMMETENRLHFTIKDPANRRYEVP |
| | | LETPRVHSRAPSPLYSVEFSEEPFGVIVHRQLDGRVLLNTTVAPLE |
| | | FADQFLQLSTSLPSQYITGLAEHLSPLMLSTSWTRITLWNRDLAPT |
| | | PGANLYGSHPFYLALEDGGSAHGVFLLNSNAMDVVLQPSPALSWRS |
| | | TGGILDVYIFLGPEPKSVVQQYLDVVGYPFMPPYWGLGFHLCRWGY |
| | | SSTAITRQVVENMTRAHFPLDVQWNDLDYMDSRRDETENKDGERDE |
| | | PAMVQELHQGGRRYMMIVDPAISSSGPAGSYRPYDEGLRRGVFITN |
| | | ETGQPLIGKVWPGSTAFPDFTNPTALAWWEDMVAEFHDQVPFDGMW |
| | | IDMNEPSNFIRGSEDGCPNNELENPPYVPGVVGGTLQAATICASSH |
| | | QFLSTHYNLHNLYGLTEAIASHRALVKARGTRPFVISRSTFAGHGR |
| | | YAGHWTGDVWSSWEQLASSVPEILQFNLLGVPLVGADVCGELGNTS |
| | | EELCVRWTOLGAFYPFMRNHNSLLSLPQEPYSESEPAQQAMRKALT |
| | | LRYALLPHLYTLFHQAHVAGETVARPLFLEFPKDSSTWTVDHQLLW |
| | | GEALLITPVLQAGKAEVTGYFPLGTWYDLQTVPIEALGSLPPPPAA |
| | | PREPAIHSEGQWVTLPAPLDTINVHLRAGYIIPLQGPGLTTTESRQ |
| | | QPMALAVALTKGGEARGELFWDDGESLEVLERGAYTQVIFLARNNT |
| | | IVNELVRVTSEGAGLQLQKVTVLGVATAPQQVLSNGVPVSNFTYSP |
| | | DTKVLDICVSLLMGEQFLVSWC |

531v25 Fab-GAA (GAA Fused to LC C-Terminus)

| | | |
|---|---|---|
| 36 | Fab LC-GAA Fusion | DIQMTQSPSSLSASVGDRVTITCRASQDISSELNWYQQKPGKAPKL |
| | (peptide linker in | LIYYTSRLQSGVPSRFSGSGSGTDYTLTISSLQPEDFATYYCQQGN |
| | box) | TLPRTFGGGTKVEIKRTVAAPSVFIFPPSDEQLKSGTASVVCLLNN |
| | | FYPREAKVQWKVDNALQSGNSQESVTEQDSKDSTYSLSSTLT |
| | | 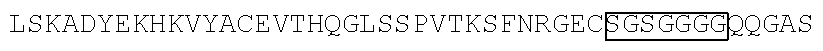 |
| | | RPGPRDAQAHPGRPRAVPTQCDVPPNSREDCAPDKAITQEQCEARG |
| | | CCYIPAKQGLQGAQMGQPWCFFPPSYPSYKLENLSSSEMGYTATLT |
| | | RTTPTFFPKDILTLRLDVMMETENRLHFTIKDPANRRYEVPL |
| | | ETPRVHSRAPSPLYSVEFSEEPFGVIVHRQLDGRVLLNTTVAPLFF |
| | | ADQFLQLSTSLPSQYITGLAEHLSPLMLSTSWTRITLWNRDLAPTP |
| | | GANLYGSHPFYLALEDGGSAHGVFLLNSNAMDVVLQPSPALSWRST |
| | | GGILDVYIFLGPEPKSVVQQYLDVVGYPFMPPYWGLGFHLCR |
| | | WGYSSTAITRQVVENMTRAHFPLDVQWNDLDYMDSRRDFTENKDGF |
| | | RDFPAMVQELHQGGRRYMMIVDPAISSSGPAGSYRPYDEGLRRGVF |
| | | ITNETGQPLIGKVWPGSTAFPDFTNPTALAWWEDMVAEFHDQVPED |
| | | GMWIDMNEPSNFIRGSEDGCPNNELENPPYVPGVVGGTLQAA |
| | | TICASSHQFLSTHYNLHNLYGLTEAIASHRALVKARGTRPFVISRS |
| | | TFAGHGRYAGHWTGDVWSSWEQLASSVPEILQFNLLGVPLVGADVC |
| | | GFLGNTSEELCVRWTQLGAFYPEMRNHNSLLSLPQEPYSESEPAQQ |
| | | AMRKALTLRYALLPHLYTLFHQAHVAGETVARPLFLEFPKDS |
| | | STWTVDHQLLWGEALLITPVLQAGKAEVTGYFPLGTWYDLQTVPIE |
| | | ALGSLPPPPAAPREPAIHSEGQWVTLPAPLDTINVHLRAGYIIPLQ |
| | | GPGLTTTESRQQPMALAVALTKGGEARGELFWDDGESLEVLERGAY |
| | | TQVIFLARNNTIVNELVRVTSEGAGLQLQKVTVLGVATAPQQ |
| | | VLSNGVPVSNFTYSPDTKVLDICVSLLMGEQFLVSWC |
| 30 | Fab HC | QVQLVQSGAEVKKPGASVKVSCKASGYTFTRYYLNWVRQAPGOGLE |
| | | WIGMIDPSVSETHYAQKFQGRATLTVDKSTSTAYMELSSLRSEDTA |
| | | VYYCSQIRLPYYYAMDSWGQGTTVTVSSASTKGPSVFPLAPSSKST |
| | | SGGTAALGCLVKDYFPEPVTVSWNSGALTSGVHTFPAVLQSS |
| | | GLYSLSSVVTVPSSSLGTQTYICNVNHKPSNTKVDKKVEPKSC |

531v25Fab-FcOL-LC-GAA (GAA Fused to LC C-Terminus)

| | | |
|---|---|---|
| 36 | Fab LC-GAA Fusion | DIQMTQSPSSLSASVGDRVTITCRASQDISSELNWYQQKPGKAPKL |
| | (peptide linker in | LIYYTSRLQSGVPSRFSGSGSGTDYTLTISSLQPEDFATYYCQQGN |
| | box) | TLPRTFGGGTKVEIKRTVAAPSVFIFPPSDEQLKSGTASVVCLLNN |
| | | FYPREAKVQWKVDNALQSGNSQESVTEQDSKDSTYSLSSTLT |
| | | 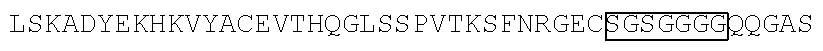 |
| | | RPGPRDAQAHPGRPRAVPTQCDVPPNSREDCAPDKAITQEQCEARG |
| | | CCYIPAKQGLQGAQMGQPWCFFPPSYPSYKLENLSSSEMGYTATLT |
| | | RTTPTFFPKDILTLRLDVMMETENRLHETIKDPANRRYEVPL |
| | | ETPRVHSRAPSPLYSVEFSEEPFGVIVHRQLDGRVLLNTTVAPLFF |
| | | ADQFLQLSTSLPSQYITGLAEHLSPLMLSTSWTRITLWNRDLAPTP |
| | | GANLYGSHPFYLALEDGGSAHGVFLLNSNAMDVVLQPSPALSWRST |
| | | GGILDVYIFLGPEPKSVVQQYLDVVGYPFMPPYWGLGFHLCR |
| | | WGYSSTAITRQVVENMTRAHFPLDVQWNDLDYMDSRRDFTENKDGF |
| | | RDFPAMVQELHQGGRRYMMIVDPAISSSGPAGSYRPYDEGLRRGVF |
| | | ITNETGQPLIGKVWPGSTAFPDFTNPTALAWWEDMVAEFHDQVPED |
| | | GMWIDMNEPSNFIRGSEDGCPNNELENPPYVPGVVGGTLQAA |
| | | TICASSHQFLSTHYNLHNLYGLTEAIASHRALVKARGTRPFVISRS |
| | | TFAGHGRYAGHWTGDVWSSWEQLASSVPEILQFNLLGVPLVGADVC |
| | | GFLGNTSEELCVRWTQLGAFYPFMRNHNSLLSLPQEPYSESEPAQQ |
| | | AMRKALTLRYALLPHLYTLFHQAHVAGETVARPLFLEFPKDS |
| | | STWTVDHQLLWGEALLITPVLQAGKAEVTGYFPLGTWYDLQTVPIE |
| | | ALGSLPPPPAAPREPAIHSEGQWVTLPAPLDTINVHLRAGYIIPLQ |
| | | GPGLTTTESRQQPMALAVALTKGGEARGELFWDDGESLEVLERGAY |
| | | TQVIFLARNNTIVNELVRVTSEGAGLQLQKVTVLGVATAPQQ |
| | | VLSNGVPVSNFTYSPDTKVLDICVSLLMGEQFLVSWC |
| 33 | HC | QVQLVQSGAEVKKPGASVKVSCKASGYTFTRYYLNWVRQAPGQGLE |
| | | WIGMIDPSVSETHYAQKFQGRATLTVDKSTSTAYMELSSLRSEDTA |
| | | VYYCSQIRLPYYYAMDSWGQGTTVTVSSASTKGPSVFPLAPSSKST |
| | | SGGTAALGCLVKDYFPEPVTVSWNSGALTSGVHTFPAVLOSS |
| | | GLYSLSSVVTVPSSSLGTQTYICNVNHKPSNTKVDKKVEPKSCDKT |
| | | HTCPPCPAPELLGGPSVFLFPPKPKDTLMISRTPEVTCVVVDVSHE |
| | | DPEVKENWYVDGVEVHNAKTKPREEQYNNASRVVSVLTVLHQDWLN |
| | | GKEYKCKVSNKALPAPIEKTISKAKGQPREPQVCTLPPSRDE |
| | | LTKNQVSLSCAVKGFYPSDIAVEWESNGQPENNYKTTPPVLDSDGS |
| | | FFLVSKLTVDKSRWQQGNVESCSVMHEALHNRFTQKSLSLSPG |
| 34 | Fc Polypeptide | DKTHTCPPCPAPELLGGPSVFLFPPKPKDTLMISRTPEVTCVVVDV |
| | | SHEDPEVKENWYVDGVEVHNAKTKPREEQYNNASRVVSVLTVLHQD |
| | | WLNGKEYKCKVSNKALPAPIEKTISKAKGQPREPQVYTLPPCRDEL |
| | | TKNQVSLWCLVKGFYPSDIAVEWESNGQPENNYKTTPPVLDS |
| | | DGSFFLYSKLTVDKSRWQQGNVFSCSVMHEALHNHYTQKSLSLSPG |

531v25FacFcOL-GAA (GAA Fused to Fc Polypeptide C-Terminus)

| | | |
|---|---|---|
| 31 | LC | DIQMTQSPSSLSASVGDRVTITCRASQDISSELNWYQQKPGKAPKL |
| | | LIYYTSRLQSGVPSRFSGSGSGTDYTLTISSLOPEDFATYYCQQGN |
| | | TLPRTFGGGTKVEIKRTVAAPSVFIFPPSDEQLKSGTASVVCLLNN |
| | | FYPREAKVQWKVDNALQSGNSQESVTEQDSKDSTYSLSSTLT |
| | | LSKADYEKHKVYACEVTHQGLSSPVTKSENRGEC |
| 33 | HC | QVQLVQSGAEVKKPGASVKVSCKASGYTFTRYYLNWVRQAPGOGLE |
| | | WIGMIDPSVSETHYAQKFQGRATLTVDKSTSTAYMELSSLRSEDTA |
| | | VYYCSQIRLPYYYAMDSWGQGTTVTVSSASTKGPSVFPLAPSSKST |
| | | SGGTAALGCLVKDYFPEPVTVSWNSGALTSGVHTFPAVLOSS |
| | | GLYSLSSVVTVPSSSLGTQTYICNVNHKPSNTKVDKKVEPKSCDKT |
| | | HTCPPCPAPELLGGPSVFLFPPKPKDTLMISRTPEVTCVVVDVSHE |
| | | DPEVKFNWYVDGVEVHNAKTKPREEQYNNASRVVSVLTVLHQDWLN |
| | | GKEYKCKVSNKALPAPIEKTISKAKGQPREPQVCTLPPSRDE |
| | | LTKNQVSLSCAVKGFYPSDIAVEWESNGQPENNYKTTPPVLDSDGS |
| | | FFLVSKLTVDKSRWQQGNVFSCSVMHEALHNRFTQKSLSLSPG |
| 37 | Fc-GAA Fusion | DKTHTCPPCPAPELLGGPSVFLFPPKPKDTLMISRTPEVTCVVVDV |
| | (peptide linker in | SHEDPEVKENWYVDGVEVHNAKTKPREEQYNNASRVVSVLTVLHQD |
| | box) | WLNGKEYKCKVSNKALPAPIEKTISKAKGQPREPQVYTLPPCRDEL |
| | | TKNQVSLWCLVKGFYPSDIAVEWESNGQPENNYKTTPPVLDS |
| | | DGSFFLYSKLTVDKSRWQQGNVFSCSVMHEALHNHYTQKSLSLSPG |
| | | 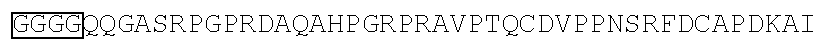 |
| | | TQEQCEARGCCYIPAKOGLQGAQMGQPWCFFPPSYPSYKLENLSSS |
| | | EMGYTATLTRTTPTFFPKDILTLRLDVMMETENRLHETIKDP |
| | | ANRRYEVPLETPRVHSRAPSPLYSVEFSEEPFGVIVHRQLDGRVLL |
| | | NTTVAPLFFADQFLQLSTSLPSQYITGLAEHLSPLMLSTSWTRITL |
| | | WNRDLAPTPGANLYGSHPFYLALEDGGSAHGVELLNSNAMDVVLQP |
| | | SPALSWRSTGGILDVYIFLGPEPKSVVQQYLDVVGYPFMPPY |
| | | WGLGFHLCRWGYSSTAITRQVVENMTRAHFPLDVQWNDLDYMDSRR |
| | | DFTFNKDGERDFPAMVQELHQGGRRYMMIVDPAISSSGPAGSYRPY |
| | | DEGLRRGVFITNETGQPLIGKVWPGSTAFPDFTNPTALAWWEDMVA |
| | | EFHDQVPFDGMWIDMNEPSNFIRGSEDGCPNNELENPPYVPG |
| | | VVGGTLQAATICASSHQFLSTHYNLHNLYGLTEAIASHRALVKARG |
| | | TRPFVISRSTFAGHGRYAGHWTGDVWSSWEQLASSVPEILQENLLG |
| | | VPLVGADVCGFLGNTSEELCVRWTQLGAFYPFMRNHNSLLSLPQEP |
| | | YSFSEPAQQAMRKALTLRYALLPHLYTLFHQAHVAGETVARP |
| | | LFLEFPKDSSTWTVDHOLLWGEALLITPVLQAGKAEVTGYFPLGTW |
| | | YDLQTVPIEALGSLPPPPAAPREPAIHSEGQWVTLPAPLDTINVHL |
| | | RAGYIIPLQGPGLTTTESRQQPMALAVALTKGGEARGELFWDDGES |
| | | LEVLERGAYTQVIFLARNNTIVNELVRVTSEGAGLQLQKVTV |
| | | LGVATAPQQVLSNGVPVSNFTYSPDTKVLDICVSLLMGEQFLVSWC |

531v25mAb-GAA (GAA Fused to one HC C-Terminus)

| | | |
|---|---|---|
| 31 | LC | DIQMTQSPSSLSASVGDRVTITCRASQDISSFLNWYQQKPGKAPKL |
| | | LIYYTSRLQSGVPSRESGSGSGTDYTLTISSLOPEDFATYYCQQGN |
| | | TLPRTFGGGTKVEIKRTVAAPSVFIFPPSDEQLKSGTASVVCLLNN |
| | | FYPREAKVQWKVDNALQSGNSQESVTEQDSKDSTYSLSSTLT |
| | | LSKADYEKHKVYACEVTHQGLSSPVTKSENRGEC |
| 38 | HC-GAA Fusion | QVQLVQSGAEVKKPGASVKVSCKASGYTFTRYYLNWVRQAPGOGLE |
| | (IgG1, knob chain, | WIGMIDPSVSETHYAQKFQGRATLTVDKSTSTAYMELSSLRSEDTA |
| | with NNAS | VYYCSQIRLPYYYAMDSWGQGTTVTVSSASTKGPSVFPLAPSSKST |
| | mutations) | SGGTAALGCLVKDYFPEPVTVSWNSGALTSGVHTFPAVLOSS |
| | (peptide linker in | GLYSLSSVVTVPSSSLGTQTYICNVNHKPSNTKVDKKVEPKSCDKT |
| | box) | HTCPPCPAPELLGGPSVELFPPKPKDTLMISRTPEVTCVVVDVSHE |
| | | DPEVKFNWYVDGVEVHNAKTKPREEQYNNASRVVSVLTVLHQDWLN |
| | | GKEYKCKVSNKALPAPIEKTISKAKGQPREPQVYTLPPCRDE |
| | | LTKNQVSLWCLVKGFYPSDIAVEWESNGQPENNYKTTPPVLDSDGS |
| | | 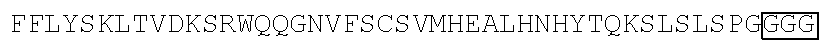 |
| | | 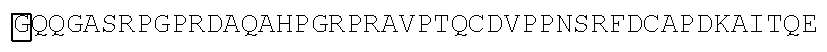 |
| | | QCEARGCCYIPAKQGLOGAQMGQPWCFFPPSYPSYKLENLSS |
| | | SEMGYTATLTRTTPTFFPKDILTLRLDVMMETENRLHFTIKDPANR |
| | | RYEVPLETPRVHSRAPSPLYSVEFSEEPFGVIVHRQLDGRVLLNTT |
| | | VAPLFFADQFLQLSTSLPSQYITGLAEHLSPLMLSTSWTRITLWNR |
| | | DLAPTPGANLYGSHPFYLALEDGGSAHGVFLLNSNAMDVVLQ |
| | | PSPALSWRSTGGILDVYIFLGPEPKSVVQQYLDVVGYPFMPPYWGL |
| | | GFHLCRWGYSSTAITRQVVENMTRAHFPLDVQWNDLDYMDSRRDET |
| | | FNKDGFRDFPAMVQELHQGGRRYMMIVDPAISSSGPAGSYRPYDEG |
| | | LRRGVFITNETGQPLIGKVWPGSTAFPDFTNPTALAWWEDMV |
| | | AEFHDQVPFDGMWIDMNEPSNFIRGSEDGCPNNELENPPYVPGVVG |
| | | GTLQAATICASSHQFLSTHYNLHNLYGLTEAIASHRALVKARGTRP |
| | | FVISRSTFAGHGRYAGHWTGDVWSSWEQLASSVPEILQFNLLGVPL |
| | | VGADVCGFLGNTSEELCVRWTQLGAFYPEMRNHNSLLSLPQE |
| | | PYSFSEPAQQAMRKALTLRYALLPHLYTLFHQAHVAGETVARPLEL |
| | | EFPKDSSTWTVDHQLLWGEALLITPVLQAGKAEVTGYFPLGTWYDL |
| | | QTVPIEALGSLPPPPAAPREPAIHSEGQWVTLPAPLDTINVHLRAG |
| | | YIIPLQGPGLTTTESRQQPMALAVALTKGGEARGELFWDDGE |
| | | SLEVLERGAYTQVIFLARNNTIVNELVRVTSEGAGLQLQKVTVLGV |
| | | ATAPQQVLSNGVPVSNFTYSPDTKVLDICVSLLMGEQFLVSWC |
| 33 | HC | QVQLVQSGAEVKKPGASVKVSCKASGYTFTRYYLNWVRQAPGQGLE |
| | (IgG1, hole chain, RF | WIGMIDPSVSETHYAQKFQGRATLTVDKSTSTAYMELSSLRSEDTA |
| | mutations) | VYYCSQIRLPYYYAMDSWGQGTTVTVSSASTKGPSVFPLAPSSKST |
| | | SGGTAALGCLVKDYFPEPVTVSWNSGALTSGVHTFPAVLOSS |
| | | GLYSLSSVVTVPSSSLGTQTYICNVNHKPSNTKVDKKVEPKSCDKT |
| | | HTCPPCPAPELLGGPSVFLFPPKPKDTLMISRTPEVTCVVVDVSHE |
| | | DPEVKFNWYVDGVEVHNAKTKPREEQYNNASRVVSVLTVLHQDWLN |
| | | GKEYKCKVSNKALPAPIEKTISKAKGQPREPQVCTLPPSRDE |
| | | LTKNQVSLSCAVKGFYPSDIAVEWESNGQPENNYKTTPPVLDSDGS |
| | | FFLVSKLTVDKSRWQQGNVFSCSVMHEALHNRFTQKSLSLSPG |

Internal Transferrin Ligand

| | | |
|---|---|---|
| 39 | Human TfR | CKGVEPKTECERLAGTESPVREEPGEDEPAARRLYWDDLKRKLSEK |
| | Ectodomain + | LDSTDFTSTIKLLNENSYVPREAGSQKDENLALYVENQFREFKLSK |
| | G142S Mutation | VWRDQHFVKIQVKDSAQNSVIIVDKNGRLVYLVENPGGYVAYSKAA |
| | (Caucasian | TVTGKLVHANFGTKKDFEDLYTPVNGSIVIVRAGKITFAEKVANAE |
| | haplotype) | SLNAIGVLIYMDQTKFPIVNAELSFFGHAHLGTGDPYTPGFPSENH |
| | (hTFRC_89- | TQFPPSRSSGLPNIPVQTISRAAAEKLEGNMEGDCPSDWKTDSTCR |
| | 760_G142S; mature | MVTSESKNVKLTVSNVLKEIKILNIFGVIKGFVEPDHYVVVGAQRD |
| | protein) | AWGPGAAKSGVGTALLLKLAQMFSDMVLKDGFQPSRSIIFASWSAG |
| | | DFGSVGATEWLEGYLSSLHLKAFTYINLDKAVLGTSNFKVSASPLL |
| | | YTLIEKTMQNVKHPVTGQFLYQDSNWASKVEKLTLDNAAFPFLAYS |
| | | GIPAVSFCFCEDTDYPYLGTTMDTYKELIERIPELNKVARAAAEVA |
| | | GQFVIKLTHDVELNLDYERYNSQLLSFVRDLNQYRADIKEMGLSLQ |
| | | WLYSARGDFFRATSRLTTDEGNAEKTDRFVMKKLNDRVMRVEYHEL |
| | | SPYVSPKESPFRHVFWGSGSHTLPALLENLKLRKONNGAFNETLER |
| | | NQLALATWTIQGAANALSGDVWDIDNEF |
| 40 | Cynomolgus Monkey | CKGVEPKTECERLAGTESPAREEPEEDFPAAPRLYWDDLKRKLSEK |
| | TfR Ectodomain | LDTTDFTSTIKLLNENLYVPREAGSQKDENLALYIENQFREFKLSK |
| | (cyTFRC_89_760; | VWRDQHFVKIQVKDSAQNSVIIVDKNGGLVYLVENPGGYVAYSKAA |
| | mature protein) | TVTGKLVHANFGTKKDFEDLDSPVNGSIVIVRAGKITFAEKVANAE |
| | | SLNAIGVLIYMDQTKFPIVKADLSFFGHAHLGTGDPYTPGFPSENH |
| | | TQFPPSQSSGLPNIPVQTISRAAAEKLFGNMEGDCPSDWKTDSTCK |
| | | MVTSENKSVKLTVSNVLKETKILNIFGVIKGFVEPDHYVVVGAQRD |
| | | AWGPGAAKSSVGTALLLKLAQMESDMVLKDGFQPSRSIIFASWSAG |
| | | DFGSVGATEWLEGYLSSLHLKAFTYINLDKAVLGTSNFKVSASPLL |
| | | YTLIEKTMQDVKHPVTGRSLYQDSNWASKVEKLTLDNAAFPFLAYS |
| | | GIPAVSFCFCEDTDYPYLGTTMDTYKELVERIPELNKVARAAAEVA |
| | | GQFVIKLTHDTELNLDYERYNSQLLLELRDLNQYRADVKEMGLSLQ |
| | | WLYSARGDFFRATSRLTTDERNAEKRDKFVMKKLNDRVMRVEYYFL |
| | | SPYVSPKESPFRHVFWGSGSHTLSALLESLKLRRONNSAFNETLER |
| | | NQLALATWTIQGAANALSGDVWDIDNEF |
| 41 | Mouse TfR | EVQLVEHHHHHHDYDIPTTENLYFQACKRVEQKEECVKLAETEETD |
| | Ectodomain | KSETMETEDVPTSSRLYWADLKTLLSEKLNSIEFADTIKQLSQNTY |
| | (mTFRC_89_764; | TPREAGSQKDESLAYYIENQFHEFKESKVWRDEHYVKIQVKSSIGQ |
| | mature protein) | NMVTIVQSNGNLDPVESPEGYVAFSKPTEVSGKLVHANFGTK |
| | | KDFEELSYSVNGSLVIVRAGEITFAEKVANAQSENAIGVLIYMDKN |
| | | KFPVVEADLALFGHAHLGTGDPYTPGEPSFNHTQFPPSQSSGLPNI |
| | | PVQTISRAAAEKLFGKMEGSCPARWNIDSSCKLELSQNQNVKLIVK |
| | | NVLKERRILNIFGVIKGYEEPDRYVVVGAQRDALGAGVAAKS |
| | | SVGTGLLLKLAQVESDMISKDGFRPSRSIIFASWTAGDEGAVGATE |
| | | WLEGYLSSLHLKAFTYINLDKVVLGTSNEKVSASPLLYTLMGKIMQ |
| | | DVKHPVDGKSLYRDSNWISKVEKLSFDNAAYPFLAYSGIPAVSFCF |
| | | CEDADYPYLGTRLDTYEALTQKVPQLNQMVRTAAEVAGQLII |
| | | KLTHDVELNLDYEMYNSKLLSFMKDLNQFKTDIRDMGLSLOWLYSA |
| | | RGDYFRATSRLTTDFHNAEKTNRFVMREINDRIMKVEYHELSPYVS |
| | | PRESPFRHIFWGSGSHTLSALVENLKLRQKNITAFNETLERNQLAL |
| | | ATWTIQGVANALSGDIWNIDNEF |

Linkers

| | | |
|---|---|---|
| 42 | Used in Fab-FcOL- | GGGG |
| | GAA and mAb-GAA | |
| | Constructs | |
| 43 | Used in Fab-GAA | SGSGGGG |
| | and Fab-FcOL-LC- | |
| | GAA Constructs | |
| 44 | Linker | (GSGGS) n, n [symbol] 1 |
| 45 | Linker | (GGGGS) n, n [symbol] 1 |
| 46 | Linker | (GGGS) n, n [symbol] 1 |
| 47 | Linker | AAEPKSS |
| 48 | Linker | AAEPKSSDKTHTCPPCP |
| 49 | Linker | GGGGDKTHTCPPCP |
| 50 | HCDRI Consensus | X₁YTFTRYX₂, optionally wherein X₁ may be G or |
| | | D, and X2 may be W or Y |
| 51 | HCDR2 Consensus | IDPSVSEX₃, optionally wherein X₃ may be T or C |
| 52 | LCDRI Consensus | QDIX₄SF, optionally wherein X₄ may be S or N |
| 53 | NNAS mutations | NNAS |
| 54 | Holo-transferrin | MRLAVGALLVCAVLGLCLAVPDKTVRWCAVSEHEATKCQSFRDHMK |
| | (signal peptide | SVIPSDGPSVACVKKASYLDCIRAIAANEADAVTLDAGLVYDAYLA |
| | underlined) | PNNLKPVVAEFYGSKEDPQTFYYAVAVVKKDSGFQMNQLRGKKSCH |
| | | TGLGRSAGWNIPIGLLYCDLPEPRKPLEKAVANFFSGSCAPCADGT |
| | | DFPQLCQLCPGCGCSTLNQYFGYSGAFKCLKDGAGDVAFVKHSTIF |
| | | ENLANKADRDQYELLCLDNTRKPVDEYKDCHLAQVPSHTVVARSMG |
| | | GKEDLIWELLNQAQEHFGKDKSKEFQLESSPHGKDLLFKDSAHGEL |
| | | KVPPRMDAKMYLGYEYVTAIRNLREGTCPEAPTDECKPVKWCALSH |
| | | HERLKCDEWSVNSVGKIECVSAETTEDCIAKIMNGEADAMSLDGGF |
| | | VYIAGKCGLVPVLAENYNKSDNCEDTPEAGYFAIAVVKKSASDLTW |
| | | DNLKGKKSCHTAVGRTAGWNIPMGLLYNKINHCRFDEFFSEGCAPG |
| | | SKKDSSLCKLCMGSGLNLCEPNNKEGYYGYTGAFRCLVEKGDVAFV |
| | | KHQTVPQNTGGKNPDPWAKNLNEKDYELLCLDGTRKPVEEYANCHL |
| | | ARAPNHAVVTRKDKEACVHKILRQQQHLFGSNVTDCSGNFCLERSE |
| | | TKDLLFRDDTVCLAKLHDRNTYEKYLGEEYVKAVGNLRKCSTSSLL |
| | | EACTFRRP |
| 55 | N-terminal AviTag | GSHHHHHHHHGGGGSLNDIFEAQKIEWHGS |
| 56 | 531v25 CH1- | QVQLVQSGAEVKKPGASVKVSCKASGYTFTRYYLNWVRQAPGOGLE |
| | NNAS/dKiH-hole/FF | WIGMIDPSVSETHYAQKFQGRATLTVDKSTSTAYMELSSLRSEDTA |
| | Fc with A114C | VYYCSQIRLPYYYAMDSWGQGTTVTVSSCSTKGPSVFPLAPSSKST |
| | 531v25 Fab-FcOL | SGGTAALGCLVKDYFPEPVTVSWNSGALTSGVHTFPAVLQSSGLYS |
| | HC | LSSVVTVPSSSLGTQTYICNVNHKPSNTKVDKKVEPKSCDKTHTCP |
| | (IgG1, with NNAS, | PCPAPELLGGPSVFLFPPKPKDTLMISRTPEVTCVVVDVSHEDPEV |
| | hole, RF, and A114C | KFNWYVDGVEVHNAKTKPREEQYNNASRVVSVLTVLHQDWLNGKEY |
| | mutations) | KCKVSNKALPAPIEKTISKAKGQPREPQVCTLPPSRDELTKNQVSL |
| | | SCAVKGFYPSDIAVEWESNGQPENNYKTTPPVLDSDGSFFLVSKLT |
| | | VDKSRWQQGNVFSCSVMHEALHNRFTQKSLSLSPG |
| 57 | MALATI-ASO | GCATTCTAATAGCAGC |
| 58 | β-actin forward | GTACGACCAGAGGCATACAG |
| | primer | |
| 59 | β-actin reverse | ACCGTGAAAAGATGACCCAG |
| | primer | |
| 60 | TaqMan qPCR | ACCTTCAAC |
| | primer | |
| 61 | TaqMan qPCR | ACCCCAGCCATGTA |
| | primer | |

1-34. (canceled)

35. An anti-human transferrin receptor (TfR) antibody or an antigen-binding fragment thereof, wherein the antibody or antigen-binding fragment comprises:
a) a heavy chain CDR (HCDR) 1 comprising GYTFTRYY (SEQ ID NO: 26), or GYTFTRYW (SEQ ID NO: 27), or DYTFTRYW (SEQ ID NO: 5),
an HCDR2 comprising IDPSVSET (SEQ ID NO: 28) or IDPSVSEC (SEQ ID NO: 6), and
an HCDR3 comprising SQIRLPYYYAMDS (SEQ ID NO: 7); and
a light chain CDR (LCDR) 1 comprising QDISSF (SEQ ID NO: 29) or QDINSF (SEQ ID NO: 9), and
an LCDR2 comprising YTS (SEQ ID NO: 10); or
b) a heavy chain CDR (HCDR) 1 comprising GYTFTRYY (SEQ ID NO: 26), or GYTFTRYW (SEQ ID NO: 27), or DYTFTRYW (SEQ ID NO: 5),
an HCDR2 comprising IDPSVSET (SEQ ID NO: 28) or IDPSVSEC (SEQ ID NO: 6), and
an HCDR3 comprising SQIRLPYYYAMDS (SEQ ID NO: 7); and
a light chain CDR (LCDR) 1 comprising QDISSF (SEQ ID NO: 29) or QDINSF (SEQ ID NO: 9),
an LCDR2 comprising YTS (SEQ ID NO: 10), and
an LCDR3 comprising QQGNTLPRT (SEQ ID NO: 11).

36. The antibody or antigen-binding fragment of claim 35, comprising
a) HCDR1-3 comprising SEQ ID NOs: 26, 28, and 7, respectively, and LCDR1-2 comprising SEQ ID NOs: 29 and 10, respectively;
b) HCDR1-3 comprising SEQ ID NOs: 5-7, respectively, and LCDR1-2 comprising SEQ ID NOs: 9 and 10, respectively;
c) HCDR1-3 comprising SEQ ID NOs: 26, 28, and 7, respectively, and LCDR1-3 comprising SEQ ID NOs: 29, 10, and 11, respectively; or
d) HCDR1-3 comprising SEQ ID NOs: 5-7, respectively, and LCDR1-3 comprising SEQ ID NOs: 9-11, respectively.

37. The antibody or antigen-binding fragment of claim 35, comprising a V_{H} and a V_{L} comprising
SEQ ID NOs: 21 and 25,
SEQ ID NOs: 21 and 24,
SEQ ID NOs: 21 and 23,
SEQ ID NOs: 19 and 23,
SEQ ID NOs: 18 and 23,
SEQ ID NOs: 17 and 23,
SEQ ID NOs: 21 and 22,
SEQ ID NOs: 20 and 22,
SEQ ID NOs: 18 and 22,
SEQ ID NOs: 17 and 22, or
SEQ ID NOs: 4 and 8,
respectively.

38. The antibody of claim 35, wherein the antibody is of human isotype subclass IgG1, IgG2, or IgG4 and comprises one or more mutations selected from
S298N, T299A, and Y300S mutations that reduce antibody-dependent cellular cytotoxicity (ADCC) and/or complement-dependent cytotoxicity (CDC),
M252Y, S254T, and T256E mutations that improve the serum half-life of the antibody,
knob-in-hole mutations of Y349C, T366S, L368A, and Y407V for hole mutations and S354C and T366W for IgG1 knob mutations; and
H435R and Y436F mutations that facilitate purification (Eu numbering).

39. The antigen-binding fragment of claim 35, wherein the antigen-binding fragment
a) is monovalent and/or comprises a Fab, or
b) is a heterotrimer comprising a heavy chain, a light chain, and a Fc polypeptide, wherein the heavy chain and the Fc polypeptide dimerize to form an Fc domain.

40. The antigen-binding fragment of claim 39, wherein the heavy chain and the Fc polypeptide of the heterotrimer comprise one or more mutations selected from
S298N, T299A, and Y300S mutations that reduce ADCC and/or CDC,
M252Y, S254T, and T256E mutations that improve the serum half-life of the antibody,
knob-in-hole mutations of Y349C, T366S, L368A, and Y407V for hole mutations and S354C and T366W for IgG1 knob mutations, and
H435R and Y436F mutations that facilitate purification (Eu numbering).

41. The antibody or antigen-binding fragment of claim 35, comprising:
a) a heavy chain comprising SEQ ID NO: 32 and a light chain comprising SEQ ID NO: 31;
b) a heavy chain comprising SEQ ID NO: 30 and a light chain comprising SEQ ID NO: 31; or
c) a heavy chain comprising SEQ ID NO: 33, a light chain comprising SEQ ID NO: 31, and an Fc polypeptide comprising SEQ ID NO: 34.

42. A TfR-binding protein comprising
a) the antibody or antigen-binding fragment of claim 35, and
b) a cargo linked to the antibody or antigen-binding fragment.

43. The TfR-binding protein of claim 42, wherein the cargo is linked to
a heavy chain,
an Fc polypeptide, if present, or
a light chain,
of the antibody or antigen-binding fragment.

44. The TfR-binding protein of claim 42, wherein the cargo is
a) an enzyme,
b) a lysosomal enzyme,
c) acid alpha-glucosidase (GAA), or
d) a polypeptide comprising SEQ ID NO: 35.

45. The TfR-binding protein of claim 44, wherein
a) the antigen-binding fragment comprises a heavy chain (HC), a light chain (LC), and an Fc polypeptide, and a human GAA sequence is fused to the C-terminus of i) the HC, ii) the LC, or iii) the Fc polypeptide, wherein the heavy chain and the Fc polypeptide dimerize to form an Fc domain,
b) the antigen-binding fragment is a Fab and a human GAA sequence is fused to the C-terminus of the HC or LC of the Fab, or
c) the antibody comprises two HCs and two LCs, and a human GAA sequence is fused to the C-terminus of one of the two HCs.

46. The TfR-binding protein of claim 45, comprising
a) an HC comprising SEQ ID NO: 33, an LC comprising SEQ ID NO: 36, and an Fc polypeptide comprising SEQ ID NO: 34;
b) an HC comprising SEQ ID NO: 33, an LC comprising SEQ ID NO: 31, and an Fc polypeptide comprising SEQ ID NO: 37;
c) an HC comprising SEQ ID NO: 30 and an LC comprising SEQ ID NO: 36; or
d) a first HC comprising SEQ ID NO: 33, a second HC comprising SEQ ID NO: 37, and two LCs each comprising SEQ ID NO: 31.

47. A pharmaceutical composition comprising the antibody or antigen-binding fragment of claim 35 and a pharmaceutically acceptable excipient.

48. One or more nucleic acid molecules or expression vectors encoding the antibody or antigen-binding fragment of claim 35.

49. A method of making a therapeutic molecule capable of crossing the blood-brain barrier of a human subject, comprising linking a therapeutic moiety of the molecule to the antibody or antigen-binding fragment of claim 35.

50. A pharmaceutical composition comprising the TfR-binding protein of claim 42 and a pharmaceutically acceptable excipient.

51. One or more nucleic acid molecules or expression vectors encoding the TfR-binding protein of claim 42.

52. A method of delivering a therapeutic molecule across the blood-brain barrier of a subject in need thereof, comprising administering the therapeutic molecule to the subject, wherein the therapeutic molecule is linked to the antibody or antigen-binding fragment of claim 35.

53. A method of treating an enzyme deficiency in a subject in need thereof, comprising administering to the subject the TfR-binding protein of claim 44.

54. A method of treating Pompe disease in a human subject in need thereof, comprising administering the antibody or antigen-binding fragment of claim 35 to the subject, wherein the antibody or antigen-binding fragment is linked to human GAA.


SEQUENCE LISTING (61 sequences)

SEQ ID NO: 1 - protein, 760 aa, Homo sapiens
  REGION 1..760: note = misc_feature - Human TfR (NP_001121620.1)
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 2 - protein, 760 aa, Macaca fascicularis
  REGION 1..760: note = misc_feature - Cynomolgus monkey (Macaca fascicularis) TfR (XP_005545315.1)
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 3 - protein, 763 aa, Mus musculus
  REGION 1..763: note = misc_feature - Mouse (Mus musculus) TfR (NP_035768.1)
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 4 - protein, 120 aa, synthetic construct
  REGION 1..120: note = Synthetic: 531-17L1 mouse VH (CDRs in box)
QVQLQQPGAELVSPGASVKLSCKASDYTFTRYWLNWVKQRPGQGLEWIGMIDPSVSECHYSQMFKDKATLTVDKSSSTAYMLLNSLTSEDSAVYYCSQIRLPYYYAMDSWGQGTSVTVSS

SEQ ID NO: 5 - protein, 8 aa, synthetic construct
  REGION 1..8: note = Synthetic: 531-17L1 HCDR1 (IMGT) / 531 vhz2 HCDR1 (IMGT)
DYTFTRYW

SEQ ID NO: 6 - protein, 8 aa, synthetic construct
  REGION 1..8: note = Synthetic: 531-17L1 HCDR2 (IMGT)
IDPSVSEC

SEQ ID NO: 7 - protein, 13 aa, synthetic construct
  REGION 1..13: note = Synthetic: 531-17L1 HCDR3 (IMGT) / 531v25 HCDR3 IMGT
SQIRLPYYYAMDS

SEQ ID NO: 8 - protein, 107 aa, synthetic construct
  REGION 1..107: note = Synthetic: 531-17L1 mouse VL (CDRs in box)
DIQMTQTPSSLSASLGDRVTISCRASQDINSFLNWYQQKPDGTVKLLIYYTSRLHSGVPSRFSGSGSGTDYSLTIGNLDQEDIATYFCQQGNTLPRTFGGGTKLELK

SEQ ID NO: 9 - protein, 6 aa, synthetic construct
  REGION 1..6: note = Synthetic: 531-17L1 LCDR1 (IMGT) / 531 vhz1 LCDR1 (IMGT)
QDINSF

SEQ ID NO: 10 - skipped

SEQ ID NO: 11 - protein, 9 aa, synthetic construct
  REGION 1..9: note = Synthetic: 531-17L1 LCDR3 (IMGT)_ / 531v25 LCDR3 (IMGT)
QQGNTLPRT

SEQ ID NO: 12 - protein, 98 aa, synthetic construct
  REGION 1..98: note = Synthetic: IGHV_23_05 germline
EVQLLESGGGLVQPGGSLRLSCAASGFTFSSYAMSWVRQAPGKGLEWVSAIYSSGSSTYYADSVKGRFTISRDNSKNTLYLQMNSLRAEDTAVYYCAK

SEQ ID NO: 13 - protein, 98 aa, synthetic construct
  REGION 1..98: note = Synthetic: IGHV1_46_01 germline
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 14 - protein, 20 aa, synthetic construct
  REGION 1..20: note = Synthetic: IGHJ6_01 germline
YYYYYGMDVWGQGTTVTVSS

SEQ ID NO: 15 - protein, 95 aa, synthetic construct
  REGION 1..95: note = Synthetic: IGKV1_39_01 germline
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 16 - protein, 12 aa, synthetic construct
  REGION 1..12: note = Synthetic: IGKJ4_01 germline
LTFGGGTKVEIK

SEQ ID NO: 17 - protein, 120 aa, synthetic construct
  REGION 1..120: note = Synthetic: 531 v1 VH (VH1A_probmotifs2) (CDRs in box)
EVQLLESGGGLVQPGGSLRLSCAASGYTFTRYWLNWVRQAPGKGLEWIGMIDPSVSETHYADSVKGRATLSVDKSKNTAYLQMNSLRAEDTAVYYCSQIRLPYYYAMDSWGQGTTVTVSS

SEQ ID NO: 18 - protein, 120 aa, synthetic construct
  REGION 1..120: note = Synthetic: 531 v2 VH (VH1A_probmotifs4) (CDRs in box)
EVQLLESGGGLVQPGGSLRLSCAASGYTFTRYYLNWVRQAPGKGLEWIGMIDPSVSETHYADSVKGRATLSVDKSKNTAYLQMNSLRAEDTAVYYCSQIRLPYYYAMDSWGQGTTVTVSS

SEQ ID NO: 19 - protein, 120 aa, synthetic construct
  REGION 1..120: note = Synthetic: 531 v3 VH (VH2A) (CDRs in box)
QVQLVQSGAEVKKPGASVKVSCKASDYTFTRYWLNWVRQAPGQGLEWIGMIDPSVSETHYAQKFQGRATLTVDKSTSTAYMELSSLRSEDTAVYYCSQIRLPYYYAMDSWGQGTTVTVSS

SEQ ID NO: 20 - protein, 120 aa, synthetic construct
  REGION 1..120: note = Synthetic: 531 v4 VH (VH2B) (CDRs in box)
QVQLVQSGAEVKKPGASVKVSCKASDYTFTRYWLNWVRQAPGQGLEWMGMIDPSVSETHYAQKFQGRVTMTRDTSTSTVYMELSSLRSEDTAVYYCSQIRLPYYYAMDSWGQGTTVTVSS

SEQ ID NO: 21 - protein, 120 aa, synthetic construct
  REGION 1..120: note = Synthetic: 531 v5 VH (VH2A_motifs4) [VH used for 531v25] (CDRs in box)
QVQLVQSGAEVKKPGASVKVSCKASGYTFTRYYLNWVRQAPGQGLEWIGMIDPSVSETHYAQKFQGRATLTVDKSTSTAYMELSSLRSEDTAVYYCSQIRLPYYYAMDSWGQGTTVTVSS

SEQ ID NO: 22 - protein, 107 aa, synthetic construct
  REGION 1..107: note = Synthetic: 531 v1 VL (VL3A) (CDRs in box)
DIQMTQSPSSLSASVGDRVTITCRASQDINSFLNWYQQKPGKAVKLLIYYTSRLQSGVPSRFSGSGSGTDYTLTISSLQPEDFATYFCQQGNTLPRTFGGGTKVEIK

SEQ ID NO: 23 - protein, 107 aa, synthetic construct
  REGION 1..107: note = Synthetic: 531 v2 VL (VL3A_motifs1) (CDRs in box)
DIQMTQSPSSLSASVGDRVTITCRASQDISSFLNWYQQKPGKAVKLLIYYTSRLQSGVPSRFSGSGSGTDYTLTISSLQPEDFATYFCQQGNTLPRTFGGGTKVEIK

SEQ ID NO: 24 - protein, 107 aa, synthetic construct
  REGION 1..107: note = Synthetic: 531 v3 VL (VL3B_motifs1) (CDRs in box)
DIQMTQSPSSLSASVGDRVTITCRASQDISSFLNWYQQKPGKAVKLLIYYTSRLQSGVPSRFSGSGSGTDFTLTISSLQPEDFATYFCQQGNTLPRTFGGGTKVEIK

SEQ ID NO: 25 - protein, 107 aa, synthetic construct
  REGION 1..107: note = Synthetic: 531 v4 VL (VL3C_motifs1) [VL used for 531v25] (CDRs in box)
DIQMTQSPSSLSASVGDRVTITCRASQDISSFLNWYQQKPGKAPKLLIYYTSRLQSGVPSRFSGSGSGTDYTLTISSLQPEDFATYYCQQGNTLPRTFGGGTKVEIK

SEQ ID NO: 26 - protein, 8 aa, synthetic construct
  REGION 1..8: note = Synthetic: 531v25 HCDR1 (IMGT)
GYTFTRYY

SEQ ID NO: 27 - protein, 8 aa, synthetic construct
  REGION 1..8: note = Synthetic: 531 vhz1 HCDR1 (IMGT)
GYTFTRYW

SEQ ID NO: 28 - protein, 8 aa, synthetic construct
  REGION 1..8: note = Synthetic: 531v25 HCDR2 (IMGT)
IDPSVSET

SEQ ID NO: 29 - protein, 6 aa, synthetic construct
  REGION 1..6: note = Synthetic: 531v25 LCDR1 (IMGT)
QDISSF

SEQ ID NO: 30 - protein, 223 aa, synthetic construct
  REGION 1..223: note = Synthetic: Construct 1 531v25 Fab HC
QVQLVQSGAEVKKPGASVKVSCKASGYTFTRYYLNWVRQAPGQGLEWIGMIDPSVSETHYAQKFQGRATLTVDKSTSTAYMELSSLRSEDTAVYYCSQIRLPYYYAMDSWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 31 - protein, 214 aa, synthetic construct
  REGION 1..214: note = Synthetic: Construct 1 531v25 Fab LC (Kappa); Construct 2 531v25 mAb LC; Construct 3 531v25 Fab-FcOL LC
DIQMTQSPSSLSASVGDRVTITCRASQDISSFLNWYQQKPGKAPKLLIYYTSRLQSGVPSRFSGSGSGTDYTLTISSLQPEDFATYYCQQGNTLPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 449 aa, synthetic construct
  REGION 1..449: note = Synthetic: Construct 2 531v25 mAb HC (IgG1, with NNAS mutations)
QVQLVQSGAEVKKPGASVKVSCKASGYTFTRYYLNWVRQAPGQGLEWIGMIDPSVSETHYAQKFQGRATLTVDKSTSTAYMELSSLRSEDTAVYYCSQIRLPYYYAMDSWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNNASRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 449 aa, synthetic construct
  REGION 1..449: note = Synthetic: Construct 3 531v25 Fab-FcOL HC (IgG1, with NNAS, hole, and RF mutations); HC; HC (IgG1, hole chain, RF mutations)
QVQLVQSGAEVKKPGASVKVSCKASGYTFTRYYLNWVRQAPGQGLEWIGMIDPSVSETHYAQKFQGRATLTVDKSTSTAYMELSSLRSEDTAVYYCSQIRLPYYYAMDSWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNNASRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 34 - protein, 226 aa, synthetic construct
  REGION 1..226: note = Synthetic: Construct 3 531v25 Fab-FcOL Fc Polypeptide (IgG1, with knob mutations); Fc Polypeptide
DKTHTCPPCPAPELLGGPSVFLFPPKPKDTLMISRTPEVTCVVVDVSHEDPEVKFNWYVDGVEVHNAKTKPREEQYNNASRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 896 aa, unidentified
  REGION 1..896: note = Exemplary human GAA (alglucosidase alfa)
QQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 36 - protein, 1117 aa, synthetic construct
  REGION 1..1117: note = Synthetic: Fab LC-GAA Fusion (peptide linker in box)
DIQMTQSPSSLSASVGDRVTITCRASQDISSFLNWYQQKPGKAPKLLIYYTSRLQSGVPSRFSGSGSGTDYTLTISSLQPEDFATYYCQQGNTLPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGSGGGGQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 37 - protein, 1126 aa, synthetic construct
  REGION 1..1126: note = Synthetic: Fc-GAA Fusion (peptide linker in box)
DKTHTCPPCPAPELLGGPSVFLFPPKPKDTLMISRTPEVTCVVVDVSHEDPEVKFNWYVDGVEVHNAKTKPREEQYNNASRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 38 - protein, 1349 aa, synthetic construct
  REGION 1..1349: note = Synthetic: HC-GAA Fusion (IgG1, knob chain, with NNAS mutations) (peptide linker in box)
QVQLVQSGAEVKKPGASVKVSCKASGYTFTRYYLNWVRQAPGQGLEWIGMIDPSVSETHYAQKFQGRATLTVDKSTSTAYMELSSLRSEDTAVYYCSQIRLPYYYAMDSWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNNASRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 39 - protein, 672 aa, synthetic construct
  REGION 1..672: note = Synthetic: Human TfR Ectodomain + G142S Mutation (Caucasian haplotype) (hTFRC_89-760_G142S; mature protein)
CKGVEPKTECERLAGTESPVREEPGEDFPAARRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 40 - protein, 672 aa, synthetic construct
  REGION 1..672: note = Synthetic: Cynomolgus Monkey TfR Ectodomain (cyTFRC_89_760; mature protein)
CKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 41 - protein, 701 aa, synthetic construct
  REGION 1..701: note = Synthetic: Mouse TfR Ectodomain (mTFRC_89_764; mature protein)
EVQLVEHHHHHHDYDIPTTENLYFQACKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 42 - protein, 4 aa, synthetic construct
  REGION 1..4: note = Synthetic: Used in Fab-FcOL- 6bis and mAb GAA Constructs
GGGG

SEQ ID NO: 43 - protein, 7 aa, synthetic construct
  REGION 1..7: note = Synthetic: Used in Fab and Fab-FcOL-4ter GAA Constructs
SGSGGGG

SEQ ID NO: 44 - protein, 5 aa, synthetic construct
  REGION 1..5: note = Synthetic: Linker
  REPEAT 1..5: note = "GSGGS" can repeat indefinitely
GSGGS

SEQ ID NO: 45 - protein, 5 aa, synthetic construct
  REGION 1..5: note = Synthetic: Linker
  REPEAT 1..5: note = "GGGGS" can repeat indefinitely
  REPEAT 1..5: note = "GGGGS" can repeat indefinitely
GGGGS

SEQ ID NO: 46 - protein, 4 aa, synthetic construct
  REGION 1..4: note = Synthetic: Linker
  REPEAT 1..4: note = "GGGS" can repeat indefinitely
GGGS

SEQ ID NO: 47 - protein, 7 aa, synthetic construct
  REGION 1..7: note = Synthetic: Linker
AAEPKSS

SEQ ID NO: 48 - protein, 17 aa, synthetic construct
  REGION 1..17: note = Synthetic: Linker
AAEPKSSDKTHTCPPCP

SEQ ID NO: 49 - protein, 14 aa, synthetic construct
  REGION 1..14: note = Synthetic: Linker
GGGGDKTHTCPPCP

SEQ ID NO: 50 - protein, 8 aa, synthetic construct
  REGION 1..8: note = Synthetic: HCDR1 Consensus
  VARIANT 1: note = X may be G or D
  VARIANT 8: note = X may be W or Y
XYTFTRYX

SEQ ID NO: 51 - protein, 8 aa, synthetic construct
  REGION 1..8: note = Synthetic: HCDR2 Consensus
  VARIANT 8: note = X may be T or C
IDPSVSEX

SEQ ID NO: 52 - protein, 6 aa, synthetic construct
  REGION 1..6: note = Synthetic: LCDR1 Consensus
  VARIANT 4: note = X may be S or N
QDIXSF

SEQ ID NO: 53 - protein, 4 aa, synthetic construct
  REGION 1..4: note = Synthetic: NNAS mutations
NNAS

SEQ ID NO: 54 - protein, 698 aa, synthetic construct
  REGION 1..698: note = Synthetic: Holotransferrin
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 55 - protein, 30 aa, synthetic construct
  REGION 1..30: note = Synthetic: N-terminal AviTag
GSHHHHHHHHGGGGSLNDIFEAQKIEWHGS

SEQ ID NO: 56 - protein, 449 aa, synthetic construct
  REGION 1..449: note = Synthetic: 531v25 CH1-NNAS/dKiH-hole/FF Fc with A114C 531v25 Fab-FcOL HC (IgG1, with NNAS, hole, RF, and A114C mutations)
QVQLVQSGAEVKKPGASVKVSCKASGYTFTRYYLNWVRQAPGQGLEWIGMIDPSVSETHYAQKFQGRATLTVDKSTSTAYMELSSLRSEDTAVYYCSQIRLPYYYAMDSWGQGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNNASRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 57 - DNA, 16 bp, synthetic construct
  misc_feature 1..16: note = Synthetic: MALAT1-ASO
  source 1..16: mol_type = other DNA
gcattctaatagcagc

SEQ ID NO: 58 - DNA, 20 bp, synthetic construct
  misc_feature 1..20: note = Synthetic: B-actin forward primer
  source 1..20: mol_type = other DNA
gtacgaccagaggcatacag

SEQ ID NO: 59 - DNA, 20 bp, synthetic construct
  misc_feature 1..20: note = Synthetic: B-actin reverse primer
  source 1..20: mol_type = other DNA
accgtgaaaagatgacccag

SEQ ID NO: 60 - skipped

SEQ ID NO: 61 - DNA, 14 bp, synthetic construct
  misc_feature 1..14: note = Synthetic: TaqMan qPCR primer
  source 1..14: mol_type = other DNA
accccagccatgta